(19) United States
(12) Patent Application Publication
(10) Pub. No.: US 2011/0150861 A1
(43) Pub. Date: Jun. 23, 2011

(54) SORF CONSTRUCTS AND MULTIPLE GENE EXPRESSION

(75) Inventors: Gerald R. Carson, Belmont, MA (US); Wendy R. Gion, Charlton, MA (US); Yune Z. Kunes, Winchester, MA (US); Walter F. Leise, III, Hawthorn Woods, IL (US); Rachel A. Davis-Taber, Sturbridge, MA (US); Emma Fung, Northborough, MA (US)

(73) Assignee: Abbott Laboratories, Abbott Park, IL (US)

(21) Appl. No.: 12/914,556

(22) Filed: Oct. 28, 2010

Related U.S. Application Data
(60) Provisional application No. 61/256,544, filed on Oct. 30, 2009.

(51) Int. Cl. A61K 39/395; C12N 15/63; C12N 1/00; C12N 1/21; C12N 1/11; C12N 5/10; C12N 1/19; C12N 1/15; C12P 21/00; C07K 16/00; C07K 16/24; A61K 38/00; A61P 43/00

(52) U.S. Cl. 424/130.1; 435/320.1; 435/243; 435/252.33; 435/258.1; 435/419; 435/325; 435/254.11; 435/349; 435/348; 435/358; 435/254.2; 435/254.21; 435/69.1; 435/69.6; 530/387.1; 530/389.2; 514/1.1

(57) ABSTRACT

Embodiments of the invention relate to vector constructs and methods for expression of polypeptides including multimeric products such as therapeutic antibodies. Particular constructs allow for the generation of expression products from a single open reading frame (sORF). An embodiment provides an isolated or purified expression vector for generating one or more recombinant protein products comprising a single open reading frame insert; said insert comprising a signal peptide nucleic acid sequence encoding a signal peptide; a first nucleic acid sequence encoding a first polypeptide; a first intervening nucleic acid sequence encoding a first protein cleavage site, wherein said first protein cleavage site is provided by an intein segment of a Ion protease gene of Pyrococcus or a klbA gene of Pyrococcus or Methanococcus, or a modified intein segment derived therefrom; and a second nucleic acid sequence encoding a second polypeptide. Certain embodiments of constructs and methods employ an intein segment of a Ion protease gene of Pyrococcus abyssi, Pyrococcus furiosus, or Pyrococcus horikoshii OT3; or an intein segment of a klbA gene of Pyrococcus abyssi, Pyrococcus furiosus, or Methanococcus jannaschii; or other intein segment.

### CROSS-REFERENCES TO RELATED APPLICATIONS

This application claims the benefit of US Provisional Patent Application Ser. No. 61/256,544 filed Oct. 30, 2009 by Gerald R. Carson et al., which is incorporated herein by reference in entirety.

### STATEMENT ON FEDERALLY SPONSORED RESEARCH OR DEVELOPMENT

Not applicable

### REFERENCE TO SEQUENCE LISTING, A TABLE, OR A COMPUTER PROGRAM LISTING COMPACT DISK APPENDIX

Not Applicable (sequence listing provided but not as compact disk appendix)

### BACKGROUND

In the field of recombinant expression technology, the achievement of high production levels of desired protein products and the ability to generate products of desired purity represent ongoing challenges. Such challenges are particularly relevant for protein products including biological therapeutics which are antibodies, but advances in this field also have relevance to other biologicals. Certain embodiments of the present invention at least in part address one or more aspects of these challenges.

### SUMMARY

The following abbreviations are applicable: ORF, open reading frame; sORF, single open reading frame; MW, molecular weight; HC or H, immunoglobulin heavy chain; LC or L, immunoglobulin light chain; pab, Pyrococcus abyssi; pfu, Pyrococcus furiosus; pho, Pyrococcus horikoshii OT3; aa or AA, amino acid(s); SP, signal peptide; LCSP, light chain signal peptide; MTX, methotrexate.

Embodiments of the invention generally relate to expression cassettes, vector constructs, recombinant host cells and methods for the recombinant expression and processing, including post-translational processing, of recombinant polyproteins and pre-proteins. In embodiments, one or more expressed products are immunoglobulins.

In embodiments, the expression vectors comprise one or more intein segments. In embodiments, the intein segments are derived from one or more Ion inteins of organisms Pyrococcus abyssi, Pyrococcus furiosus, and Pyrococcus horikoshii OT3.

In embodiments, the architecture of a construct is configured with respect to the order and presence or absence of certain elements. In an embodiment, the order of certain vector gene segments is HL, where H and L indicate an immunoglobulin heavy and light chain respectively. In another embodiment, the order is LH. In a particular embodiment, the construct has a design labeled as (−) where the minus sign indicates that the construct has one signal peptide at the beginning of the ORF and a methionine inserted between the last amino acid of the intein and the first amino acid of the second protein subunit, e.g., a mature antibody chain following the intein. In a particular embodiment, the construct has a design labeled as (+) where the plus sign indicates the presence of a first signal peptide at the beginning of the ORF and a second signal peptide at the beginning of the second protein subunit downstream of the intein. In a particular embodiment the configuration is HL(−).

In embodiments, the invention provides sORF (single open reading frame) construct designs capable of producing levels of protein expression which are greater than 2, 5, 10, 20, 30, 40, or 50 microgams per ml of secreted product when measured in culture supernatants from experiments under transient expression conditions. In embodiments, the invention provides sORF constructs capable of producing levels of protein expression which are greater than 20 micrograms per ml per day when measured in culture supernatants from experiments with conditions using a stable CHO (Chinese hamster ovary) cell expression system. In an embodiment, the expression level (pg/ml/day) is in the range of 1 to 24, greater than 10, or greater than 20. In a particular embodiment, the expression level is 24 μg/ml/day. In embodiments, the protein expression is of secreted antibody which has self-assembled into a multimeric unit of heavy and light chains. In embodiments, the antibody is of an IgG isotype.

In an embodiment, the invention provides an isolated or purified expression vector for generating one or more recombinant protein products comprising a single open reading frame insert; said insert comprising:
(a) a signal peptide nucleic acid sequence encoding a signal peptide;
(b) a first nucleic acid sequence encoding a first polypeptide;
(c) a first intervening nucleic acid sequence encoding a first protein cleavage site, wherein said first protein cleavage site is provided by an intein segment of a Ion protease gene of Pyrococcus or a klbA gene of Pyrococcus or Methanococcus, or a modified intein segment derived therefrom; and
(d) a second nucleic acid sequence encoding a second polypeptide;
wherein said first intervening nucleic acid sequence encoding said first protein cleavage site is operably positioned between said first nucleic acid sequence and said second nucleic acid sequence;
wherein said signal peptide nucleic acid sequence encoding said signal peptide is operably positioned before said first nucleic acid sequence; and
wherein said expression vector is capable of expressing a single open reading frame polypeptide cleavable at said first protein cleavage site.

For clarity in the context of embodiments comprising various intervening segments and methods, an intervening nucleic acid sequence encoding a protein cleavage site can be such that the intervening nucleic acid sequence encodes at least a first protein cleavage site. In canonical inteins, for example, the cleavage reaction generally proceeds in an autoprocessive and rapid manner. A further explanation is in part dependent on the understanding of underlying mechanisms. From the post-processing perspective looking at the extein components, it can be understood that there is a first protein cleavage site and a second protein cleavage site toward the N-terminus and C-terminus of the intein segment, respectively. The designation of the cleavage sites is not intended to necessarily correspond to the order in which cleavage reactions may occur, and it is recognized that there can be a perception of the cleavage reaction to be a single and relatively coordinated event at one cleavage reaction site even if there is an appreciation of kinetically distinct steps in a given mechanism. This description also provides for embodiments of compositions and methods, as would be understood in the art, with intervening segments comprising one or more cleavage sites.

Again depending on the understanding of processing mechanisms, a segment comprising one cleavage site or two cleavage sites can each allow for partial or complete excision of an intervening segment.

In an embodiment, an intervening nucleic acid sequence further encodes a second protein cleavage site.

In an embodiment of an expression vector, the first protein cleavage site is provided by an intein segment of a Ion protease gene of Pyrococcus abyssi, Pyrococcus furiosus, or Pyrococcus horikoshii OT3; or an intein segment of a klbA gene of Pyrococcus abyssi, Pyrococcus furiosus, or Methanococcus jannaschii; or a modified intein segment derived respectively therefrom.

In an embodiment, the intein segment or modified intein segment encodes a penultimate residue which is a lysine, serine or not a histidine. In an embodiment, the intein segment or modified intein segment is capable of cleavage but not complete ligation of said first polypeptide to said second polypeptide.

In an embodiment, the first protein cleavage site is provided by an intein segment comprising a sequence selected from the group consisting of SEQ ID NO: 1, 3, 4, 6, 7, 55, 35, 37, and 39 and modified intein segments derived therefrom.

In an embodiment, the first polypeptide and second polypeptide are capable of multimeric assembly. In an embodiment, at least one of said first polypeptide and second polypeptide are capable of extracellular secretion. In an embodiment, at least one of said first polypeptide and second polypeptide are of mammalian origin. In an embodiment, the first polypeptide comprises an immunoglobulin heavy chain or functional fragment thereof, and said second polypeptide comprises an immunoglobulin light chain or functional fragment thereof, and said first polypeptide is upstream of (5′ to) said second polypeptide.

In an embodiment of an expression vector, the vector comprises only one signal peptide nucleic acid sequence.

In an embodiment, an expression vector further comprises a third nucleic acid sequence encoding a third polypeptide, and a second intervening nucleic acid sequence encoding a second protein cleavage site; wherein the second intervening nucleic acid sequence and third nucleic acid sequence, in that order, are operably positioned after said second nucleic acid sequence.

In an embodiment of an expression vector, the first and said second polypeptide comprise a functional antibody or other antigen recognition molecule; with an antigen specificity directed to binding an antigen selected from the group consisting of: tumor necrosis factor-a, erythropoietin receptor, RSV, EL/selectin, interleukin-1, interleukin-12, interleukin-13, interleukin-17, interleukin-18, interleukin-23, interleukin-33, CD81, CD19, IGF1, IGF2, EGFR, CXCL-13, GLP-1R, prostaglandin E2, and amyloid beta.

In an embodiment of the invention, for an expression vector the first and second polypeptides comprise a pair of immunoglobulin chains from an antibody of D2E7, EL246, ABT-007, ABT-325, or ABT-874. In an embodiment, the first and second polypeptide are each independently selected from an immunoglobulin heavy chain or an immunoglobulin light chain segment from an analogous segment of D2E7, EL246, ABT-007, ABT-325, ABT-874, or other antibody.

In an embodiment, an expression vector further comprises a promoter regulatory element for said insert. In an embodiment, the promoter regulatory element is inducible or constitutive. In an embodiment, the promoter regulatory element is tissue specific. In an embodiment, the promoter comprises an adenovirus major late promoter.

In an embodiment, the invention provides a host cell comprising a vector described herein. In an embodiment, the host cell is a prokaryotic cell. In an embodiment, the host cell is Escherichia coli. In an embodiment, the host cell is a eukaryotic cell. In an embodiment, the eukaryotic cell is selected from the group consisting of a protist cell, animal cell, plant cell, and fungal cell. In an embodiment, the eukaryotic cell is an animal cell selected from the group consisting of a mammalian cell, an avian cell, and an insect cell. In an embodiment, the host cell is a mammalian cell line. In an embodiment, the host cell is a CHO cell or a dihydrofolate reductase-deficient CHO cell. In an embodiment, the host cell is an HEK (human embryonic kidney) cell or an African green monkey kidney cell, e.g., a COS cell. In an embodiment, the host cell is a yeast cell. In an embodiment, the yeast cell is Saccharomyces cerevisiae. In a embodiment, the host cell is a Spodoptera frugiperda Sf9 insect cell.

In an embodiment, the invention provides a method for producing a recombinant polyprotein or a plurality of proteins, comprising culturing a host cell in a culture medium under conditions sufficient to allow expression of a vector protein. In an embodiment, the method further comprises recovering and/or purifying said vector protein. In an embodiment of a production method, the plurality of proteins are capable of multimeric assembly. In an embodiment, the recombinant polyprotein or plurality of proteins are biologically functional and/or therapeutic.

In an embodiment, the invention provides a method for producing a recombinant product, wherein the product is an immunoglobulin protein or functional fragment thereof, assembled antibody, or other antigen recognition molecule, comprising culturing a host cell in a culture medium under conditions sufficient to produce the recombinant product. In an embodiment, the invention provides a protein or polyprotein produced according to a method described herein. In embodiment, the invention provides an assembled immunoglobulin; assembled other antigen recognition molecule; or individual immunoglobulin chain or functional fragment thereof produced according to a method herein. In an embodiment, regarding the immunoglobulin; other antigen recognition molecule; or individual immunoglobulin chain or functional fragment thereof, there is a capability to effect or contribute to specific antigen (where an antigen may be a ligand or counterreceptor, etc.) binding to tumor necrosis factor-a, erythropoietin receptor, RSV, EL/selectin, interleukin-1, interleukin-12, interleukin-13, interleukin 17, interleukin-18, interleukin-23, interleukin-33, CD81, CD19, IGF1, IGF2, EGFR, CXCL-13, GLP-1 R, prostaglandin E2 or amyloid beta. In an embodiment, the immunoglobulin or functional fragment thereof is the immunoglobulin D2E7 or ABT-874 or the functional fragment is a fragment respectively thereof.

In an embodiment, the invention provides a pharmaceutical composition comprising a therapeutically effective amount of a protein and a pharmaceutically acceptable carrier.

In an embodiment, the invention provides an expression vector as described herein further comprising a nucleic acid sequence encoding a tag. In an embodiment of a vector construct, the intervening nucleic acid sequence additionally encodes a tag.

In an embodiment, the first and said second polypeptide comprise a functional antibody or other antigen recognition molecule; with an antigen specificity directed to binding an antigen selected from the group consisting of: tumor necrosis factor-a, erythropoietin receptor, RSV, EL/selectin, interleukin-1, interleukin-12, interleukin-13, interleukin-17, interleukin-18, interleukin-23, interleukin-33, CD81, CD19, IGF1, IGF2, EGFR, CXCL-13, GLP-1R, prostaglandin E2, and amyloid beta. In an embodiment, the first and second polypeptides comprise a pair of immunoglobulin chains from an antibody of D2E7, EL246, ABT-007, ABT-325, or ABT-874. In an embodiment, the first and second polypeptide are each independently selected from an immunoglobulin heavy chain or an immunoglobulin light chain segment from an analogous segment of D2E7, EL246, ABT-007, ABT-325, ABT-874, or other antibody.

In an embodiment, a vector further comprises a promoter regulatory element for said sORF insert. In an embodiment, said promoter regulatory element is inducible or constitutive. In an embodiment, said promoter regulatory element is tissue specific. In an embodiment, said promoter comprises an adenovirus major late promoter.

In an embodiment, a vector further comprises a nucleic acid encoding a protease capable of cleaving said first protein cleavage site. In an embodiment, said nucleic acid encoding a protease is operably positioned within said sORF insert; said expression vector further comprising an additional nucleic acid encoding a second cleavage site located between said nucleic acid encoding a protease and at least one of said first nucleic acid and said second nucleic acid.

In an embodiment, the invention provides a host cell comprising a vector described herein. In an embodiment, the host cell is a prokaryotic cell. In an embodiment, said host cell is Escherichia coli. In an embodiment, said host cell is a eukaryotic cell. In an embodiment, said eukaryotic cell is selected from the group consisting of a protist cell, animal cell, plant cell and fungal cell. In an embodiment, said eukaryotic cell is an animal cell selected from the group consisting of a mammalian cell, an avian cell, and an insect cell. In a preferred embodiment, said host cell is a CHO cell or a dihydrofolate reductase-deficient CHO cell. In an embodiment, said host cell is a COS cell. In an embodiment, said host cell is a yeast cell. In an embodiment, said yeast cell is Saccharomyces cerevisiae. In an embodiment, said host cell is an insect Spodoptera frugiperda Sf9 cell. In an embodiment, said host cell is a human embryonic kidney cell.

In an embodiment, the invention provides a method for producing a recombinant polyprotein or a plurality of proteins, comprising culturing a host cell in a culture medium under conditions sufficient to allow expression of a vector protein. In an embodiment, the method further comprises recovering and/or purifying said vector protein. In an embodiment, said plurality of proteins are capable of multimeric assembly. In an embodiment, the recombinant polyprotein or plurality of proteins are biologically functional and/or therapeutic.

In an embodiment, the invention provides a method for producing an immunoglobulin protein or functional fragment thereof, assembled antibody, or other antigen recognition molecule, comprising culturing a host cell according to claim 38 in a culture medium under conditions sufficient to produce an immunoglobulin protein or functional fragment thereof, assembled antibody, or other antigen recognition molecule.

In an embodiment, the invention provides a protein or polyprotein produced according to a method herein. In an embodiment, the invention provides an assembled immunoglobulin; assembled other antigen recognition molecule; or individual immunoglobulin chain or functional fragment thereof produced according to the methods herein. In an embodiment, the immunoglobulin; other antigen recognition molecule; or individual immunoglobulin chain or functional fragment thereof has a capability to effect or contribute to specific antigen binding to tumor necrosis factor-a, erythropoietin receptor, interleukin-18, EL/selectin or interleukin-12. In an embodiment, the immunoglobulin is D2E7 or wherein the functional fragment is a fragment of D2E7.

In an embodiment, the invention provides an expression vector, host cell with the vector, vector expression product, pharmaceutical composition, and/or method of making or using any of the foregoing, wherein the vector is the vector of any of claims 1-9 and further comprises a segment encoding a light chain signal peptide. In an embodiment, the encoded light chain signal peptide is a kappa light chain signal peptide selected from the group consisting of A17, A18, A19, A26, and H2G. In an embodiment, the encoded light chain signal peptide is VKII kappa light chain signal peptide A18, SEQ ID NO:82 (amino acid sequence MRLPAQLLGLLMLWIPGSSA).

In an embodiment, a composition of the invention is isolated or purified.

In an embodiment, a composition of the invention is a peptide compound. In an embodiment, a composition of the invention is a nucleic acid compound. In an embodiment, a peptide compound of the invention is assembled in a multimeric complex with the peptide or at least one other peptide.

In an embodiment, the invention provides a pharmaceutical formulation comprising a composition of the invention. In an embodiment, the invention provides a method of synthesizing a composition of the invention or a pharmaceutical formulation thereof. In an embodiment, a pharmaceutical formulation comprises one or more excipients, carriers, and/or other components as would be understood in the art. In an embodiment, an effective amount of a composition of the invention can be a therapeutically effective amount.

In an embodiment, a peptide composition of the invention is prepared using recombinant methodology or synthetic techniques. In an embodiment, a nucleic acid composition of the invention is prepared using recombinant methodology or synthetic techniques.

In embodiments, the invention provides methods of use in the manufacture of a medicament.

Other aspects, features and advantages of embodiments of the invention are apparent from the following description when taken in conjunction with the accompanying drawings and in the context of the field of art.

In general the terms and phrases used herein have their art-recognized meaning, which can be found by reference to standard texts, journal references and contexts known to those skilled in the art. Definitions provided herein are intended to clarify their specific use in the context of embodiments of the invention.

Without wishing to be bound by any particular theory, there can be discussion herein of beliefs or understandings of underlying principles or mechanisms relating to the invention. It is recognized that regardless of the ultimate correctness of any explanation or hypothesis, an embodiment of the invention can nonetheless be operative and useful.

### BRIEF DESCRIPTION OF THE FIGURES

FIG. 1 illustrates a schematic diagram of a sORF expression construct, pTT3 pab Ion HL(−).

FIG. 2 illustrates structures of the sORF components in expression constructs for the D2E7 antibody.

FIG. 3 illustrates results of an SDS-PAGE for protein analysis of sORF expression products. Secreted IgG molecules were purified by Protein A affinity chromatography and separated by SDS-PAGE under non-reducing (A) and reducing (B) conditions. Lanes and samples from left to right are: (Lane 1) MW reference markers; (2) control construct product; (3) Pab-lon mut A1; (4) Pab-lon mut A2; (5) pTT3 pfu Ion YP, and (6) pTT3 pfu Ion MA.

FIG. 4 illustrates results of an SDS-PAGE for protein analysis of further sORF expression products. Secreted IgGs were purified by Protein A affinity chromatography and separated by SDS-PAGE under non-reducing (A) and reducing (B) conditions. Lanes and samples from left to right are: (Lane 1) MW markers; (2) control; (3) pTT3 pfu Ion HL(−); and (4) pTT3 pfu Ion MutA.

FIG. 5 illustrates the analysis of secreted antibody produced from sORF constructions using klbA inteins. IgG products secreted from Pab-klbA HL(−) and Mja-klbA HL(−) constructs were purified by Protein A affinity chromatography and separated by SDS-PAGE under reducing (panels A, B, and C) and non-reducing (panel D) conditions. Panels A and D represent images of stained gels; panel B is an immunoblot using an antibody against human IgG1 Fc; and panel C is an immunoblot using an antibody against human kappa light chain. Lanes and samples from left to right are: (Lane 1) control; (2) Pab-klbA HL(−); and (3) Mja-klbA HL(−). The control is the same antibody produced from the expression of two separate open reading frames.

FIG. 6 illustrates the results of expression of single open reading frame constructs using the Pab klbA intein with modifications to amino acid residues at the N-terminal splicing junction. Secreted IgG proteins were purified by Protein A affinity chromatography and separated by SDS-PAGE under non-reducing and reducing conditions. Lanes and samples from left to right are: (Lane 1) MW markers; (2) The same antibody produced using conventional vector (control); (3) pTT3 Pab klba HL(−)wt; (4) pTT3 Pab klba HL(−)GC; and (5) pTT3 Pab klba HL(−)KC.

FIG. 7 illustrates a schematic diagram of a sORF expression construct, pA190-Pab-Ion HL(−), which is adapted for use as a stable expression vector in a CHO cell line system.

FIG. 8 illustrates the results of the time and frequency of reaching culture confluency for stable expression systems of sORF construct transfection clones (sORF Pab Ion constructs).

FIG. 9 illustrates schematic diagrams of structures for sORF components of transient expression constructs with light chain junction mutations. Series of constructs are designated “M1-X” (first line) and D2-X″ (second line) where X indicates any amino acid.

FIG. 10 illustrates IgG secretion results for a series of sORF constructs with light chain junction mutations based on variation of the Met1 residue.

FIG. 11 illustrates IgG secretion results for a series of sORF constructs with light chain junction mutations based on variation of the Asp2 residue.

FIG. 12 illustrates results of SDS-PAGE analysis for protein products from examples of each of the Met1 and Asp2 series of constructs with light chain junction mutations.

FIG. 13 illustrates schematic diagrams of structures for sORF components of transient expression constructs that are capable of expressing the ABT-874 antibody.

FIG. 14 illustrates certain structural motifs of inteins in the context of the location of a preferred location (dashed arrow pointing near junction of segments H and F, towards the end of the DOD endonuclease domain) of a solvent accessible loop for introduction of inserts including tags.

FIG. 15 illustrates a plasmid map of an expression construct with light chain signal peptide Al 8 for use in a transient transfection system of HEK293 cells.

FIG. 16 illustrates results of SDS-PAGE analysis of products of antibody expression constructs.

FIG. 17 illustrates the results of Western blot analysis of products of antibody expression constructs from transfected cell lines including transiently transfected HEK293 cells and stably transfected CHO cells.

FIG. 18 illustrates a plasmid map of an expression construct with light chain signal peptide Al 8 for use in a stable transfection system of CHO cells.

### DETAILED DESCRIPTION OF THE INVENTION

The invention may be further understood by the following non-limiting examples.

Certain information will be appreciated by disclosure in the art, including that according to US 20070065912 by Carson et al., Mar. 22, 2007.

The present invention provides systems, e.g., constructs and methods, for expression of a compound structure or a biologically active protein such as an enzyme, hormone (e.g., insulin), cytokine, chemokine, receptor, antibody, or other molecule. Preferably, the protein is an immunomodulatory protein such as an interleukin, a full length immunoglobulin, fragment thereof, other antigen recognition molecule as understood in the art, or other biotherapeutic molecule. An overview of such systems is in the specific context of an immunoglobulin molecule where recombinant production is based on expression of heavy and light chain coding sequences under the transcriptional control of a single promoter, wherein conversion of a single translation product (polyprotein) to the separate heavy and light chains is mediated by an intein component.

In an embodiment, either the first or second chain of the immunoglobulin polyprotein molecule may be a heavy chain or a light chain. A sequence encoding a recombinant immunoglobulin segment may be a full length coding sequence or a fragment thereof. In a specific embodiment, a second light chain coding sequence must be part of the sequence encoding the polyprotein to be processed in the practice of the present invention; i.e., taken together there are three segments comprising two light chains and one heavy chain, in any order. In particular embodiments, constructs are configured with these components and in this order: a) IgH-IgL; b) IgL-IgH; c) IgH-IgL-IgL; d) IgL-IgH-IgL; e) IgL-IgL-IgH; f) IgH-IgH-IgL; g) IgH-IgL-IgH; and/or h) IgL-IgH-IgH. In an embodiment, the hyphen can indicate the location where a cleavage site sequence is located.

Alternatively, the immunoglobulin heavy and light chain coding sequences are fused in frame to an intein coding sequence there between, with the intein either being naturally able or modified so as to lack splicing activity or the termini of the heavy and light chains designed so that splicing preferably does not occur or such that splicing occurs with poor efficiency such that unspliced antibody molecules predominate. In addition, a modified intein can further be modified still further so that there is no endonuclease region (where an endonuclease region had previously existed), with the proviso that site specific proteolytic cleavage activity remains so that the light and heavy antibody polypeptides are freed from the intervening intein portion of the primary translation product. Either the light or the heavy antibody polypeptide can be the N-extein, and either can be the C-extein.

The vector may be any recombinant vector capable of expression of a full length polyprotein, for example, an adeno-associated virus (AAV) vector, a lentivirus vector, a retrovirus vector, a replication competent adenovirus vector, a replication deficient adenovirus vector and a gutless adenovirus vector, a herpes virus vector or a nonviral vector (plasmid) or any other vector known to the art, with the choice of vector appropriate for the host cell in which the immunoglobulin or other protein(s) are expressed. Baculovirus vectors are available for expression of genes in insect cells. Numerous vectors are known to the art, and many are commercially available or otherwise readily accessible to the art.

### Regulatory Sequences Including Promoters; Host Cells

A vector for recombinant immunoglobulin or other protein expression may include any of a number of promoters known to the art, wherein the promoter is constitutive, regulatable or inducible, cell type specific, tissue-specific, or species specific. Further specific examples include, e.g., tetracycline-responsive promoters (Gossen M, Bujard H, Proc Natl Acad Sci U S A. 1992, 15; 89(12):5547-51). The vector is a replicon adapted to the host cell in which the chimeric gene is to be expressed, and it desirably also comprises a replicon functional in a bacterial cell as well, advantageously, Escherichia coli, a convenient cell for molecular biological manipulations.

The host cell for gene expression can be, without limitation, an animal cell, especially a mammalian cell, or it can be a microbial cell (bacteria, yeast, fungus, but preferably eukaryotic) or a plant cell. Particularly suitable host cells include insect cultured cells such as Spodoptera frugiperda cells, yeast cells such as Saccharomyces cerevisiae or Pichia pastoris, fungi such as Trichoderma reesei, Aspergillus, Aureobasidum and Penicillium species as well as mammalian cells such as CHO (Chinese hamster ovary), BHK (baby hamster kidney), COS, 293, 3T3 (mouse), Vero (African green monkey) cells and various transgenic animal systems, including without limitation, pigs, mice, rats, sheep, goat, cows, can be used as well. Chicken systems for expression in egg white and transgenic sheep, goat and cow systems are known for expression in milk, among others. Baculovirus, especially AcNPV, vectors can be used for the single ORF antibody expression and cleavage of the present invention, for example with expression of the sORF under the regulatory control of a polyhedrin promoter or other strong promote in an insect cell line; such vectors and cell lines are well known to the art and commercially available. Promoters used in mammalian cells can be constitutive (Herpes virus TK promoter, McKnight, Cell 31:355, 1982; SV40 early promoter, Benoist et al. Nature 290:304, 1981 Rous sarcoma virus promoter, Gorman et al. Proc. Natl. Acad. Sci. USA 79:6777, 1982; cytomegalovirus promoter, Foecking et al. Gene 45:101, 1980; mouse mammary tumor virus promoter, generally see Etcheverry in Protein Engineering: Principles and Practice, Cleland et al., eds, pp.162-181, Wiley & Sons, 1996) or regulated (metallothionein promoter, Hamer et al. J. Molec. Appl. Genet. 1:273, 1982, for example). Vectors can be based on viruses that infect particular mammalian cells, especially retroviruses, vaccinia and adenoviruses and their derivatives are known to the art and commercially available. Promoters include, without limitation, cytomegalovirus, adenovirus late, and the vaccinia 7.5K promoters. Yeast and fungal vectors (see, e.g., Van den Handel, C. et al. (1991) In: Bennett, J. W. and Lasure, L.L. (eds.), More Gene Manipulations in Fungi, Academy Press, Inc., New York, 397-428) and promoters are also well known and widely available. Enolase is a well known constitutive yeast promoter, and alcohol dehydrogenase is a well known regulated promoter.

The selection of the specific promoters, transcription termination sequences and other optional sequences, such as sequences encoding tissue specific sequences, will be determined in large part by the type of cell in which expression is desired. The may be bacterial, yeast, fungal, mammalian, insect, chicken or other animal cells.

### Signal Sequences

The coding sequence of the protein to be cleaved, proteolytically processed or self processed, which is incorporated in the vector, may further comprise one or more sequences encoding one or more signal sequences. These encoded signal sequences can be associated with one or more of the mature segments within the polyprotein. For example, the sequence encoding the immunoglobulin heavy chain leader sequence can precede the coding sequence for the heavy chain, operably linked and in frame with the remainder of the polyprotein coding sequence. Similarly, a light chain leader peptide coding sequence or other leader peptide coding sequence can be associated in frame with one or both of the immunoglobulin light chain coding sequences, with the leader sequence-chain being separated by the adjacent chain from either a self-processing site (such as 2A) or by a sequence encoding a protease recognition sequence, with the appropriate reading frame being maintained.

### Stoichiometry of Immunoglobulin Heavy and Light Chains

In many embodiments herein, immunoglobulin/antibody light chains chains (IgL) and heavy chains (IgH) are present at a vector level or at an expressed intracellular level within a host cell at about a 1:1 ratio (IgL:IgH). Whereas recombinant approaches herein and elsewhere have relied on equimolar expression of heavy and light chains (see, e.g., US Patent Publication 2005/0003482A1 or International Publication WO2004/113493), in other embodiments the present invention provides methods and expression cassettes and vectors with light and heavy chain coding sequences in a ratio of 2:1 and co-expressed with self-processing or proteolytic processing of the chains when the primary translation product is a polyprotein. In embodiments, the ratio is greater than 1:1, such as about 2:1 or greater than 2:1. In a particular embodiment, a light chain coding sequence is used at a ratio of greater than 1:1 (IgL:IgH). In a specific embodiment, the ratio of IgL:IgH is 2:1. Thus in embodiments, advantages offered by a sORF antibody expression technology include the ability to manipulate gene dosage ratios for heavy and light chains, the proximity of heavy and light chain polypeptides for multi-subunit assembly in ER, and the potential for high efficiency protein secretion.

The invention further provides host cells or stable clones of host cells transformed or infected with a vector that comprises a sequence encoding a heavy and either one or at least two light chains of an immunoglobulin (i.e., an antibody);

sequences encoding cleavage sites, such as self-processing, protease recognition sites or signal peptides there between; and may further comprise a sequence or sequences encoding an additional proteolytic cleavage site. Also included in the scope of the invention is the use of such cells or clones in generating full length recombinant immunoglobulins or fragments thereof or other biologically active proteins which are comprised of multiple subunits (e.g., two-chain or multi-chain molecules or those which are in nature produced as a pro-protein and cleaved or processed to release a precursor-derived protein and the active portion). Non-limiting examples include insulin, interleukin-18, interleukin-1, bone morphogenic protein 4, bone morphogenic protein 2, any other two chain bone morphogenic proteins, nerve growth factor, renin, chymotrypsin, transforming growth factor (3, and interleukin 1β.

In a related aspect, the invention provides a recombinant immunoglobulin molecule or fragment thereof or other protein produced by such a cell or clones, wherein the immunoglobulin comprises amino acids derived from a self processing cleavage site (such as an intein or hedgehog domain), cleavage site or signal peptide cleavage and methods, vectors and host cells for producing the same. In embodiments, the invention provides host cells containing one or more constructs as described herein.

The present invention provides single vector constructs for expression of an immunoglobulin molecule or fragment thereof and methods for in vitro or in vivo use of the same. The vectors have self-processing or other protease recognition sequences between a first and second and between a second and third immunoglobulin coding sequence, allowing for expression of a functional antibody molecule using a single promoter and transcript. Exemplary vector constructs comprise a sequence encoding a self-processing cleavage site between open reading frames and may further comprise an additional proteolytic cleavage site adjacent to the self-processing cleavage site for removal of amino acids that comprise the self-processing cleavage site following cleavage. The vector constructs find utility in methods relating to enhanced production of full length biologically active immunoglobulins or fragments thereof in vitro and in vivo. Other biologically active proteins with at least two different chains can be made using the same strategy, although it is understood that it may not be required that either chain's coding sequence be present in a ratio greater than 1 relative to the other chain's coding sequence.

Although particular compositions and methods are exemplified herein, it is understood that any of a number of alternative compositions and methods are applicable and suitable for use in practicing the invention. It will also be understood that an evaluation of the polyprotein expression cassette and vectors, host cells and methods of the invention may be carried out using procedures standard in the art. The practice of the present invention will employ, unless otherwise indicated, conventional techniques of cell biology, molecular biology (including recombinant techniques), microbiology, biochemistry and immunology, which are within the scope of those of skill in the art. Such techniques are explained fully in the literature, such as, Molecular Cloning: A Laboratory Manual, second edition (Sambrook et al., 1989); Oligonucleotide Synthesis (M. J. Gait, ed., 1984); Animal Cell Culture (R. I. Freshney, ed., 1987); Methods in Enzymology (Academic Press, Inc.); Handbook of Experimental Immunology (D. M. Weir & C. C. Blackwell, eds.); Gene Transfer Vectors for Mammalian Cells (J. M. Miller & M. P. Calos, eds., 1987); Current Protocols in Molecular Biology (F. M. Ausubel et al., eds., 1993); PCR: The Polymerase Chain Reaction, (Mullis et al., eds., 1994); and Current Protocols in Immunology (J. E. Coligan et al., eds., 1991), each of which is expressly incorporated by reference herein.

Unless otherwise indicated, all terms used herein have the same meaning as they would to one skilled in the art and the practice of the present invention will employ, conventional techniques of microbiology and recombinant DNA technology, which are within the knowledge of those of skill of the art.

The term “modified” as generally used herein in the context of a protein refers to a segment wherein at least one amino acid residue is substituted in, deleted from, or added to, the referenced molecule. Similarly, in the context of a nucleic acid the term refers to a segment wherein at least one nucleic acid subunit is substituted in, deleted from, or added to, the referenced molecule.

The term “intein” as used herein typically refers to an internal segment of a protein that facilitates its own removal and effects the joining of flanking segments known as exteins. Many examples of inteins are recognized in a variety of types of organisms, in some cases with shared structural and/or functional features. The invention is broadly able to employ inteins, and variants thereof, as appreciated to exist and further be recognized or discovered. See, e.g., Gogarten J P et al., 2002, Annu Rev Microbiol. 2002; 56:263-87; Perler, F. B. (2002), InBase, the Intein Database. Nucleic Acids Res. 30, 383-384 (also via internet at website of New England Biolabs, Inc., Ipswich, Mass.; http://www.neb.com/neb/inteins.html; Amitai G, et al., Mol Microbiol. 2003, 47(1):61-73; Gorbalenya AE, Nucleic Acids Res. 1998; 26(7): 1741-1748. Non-canonical inteins). In a protein an intein-containing unit or intein splicing unit can be understood as encompassing portions of the flanking exteins where structural aspects can contribute to reactions of cleavage, ligation, etc. The term can also be understood as a category in referring to an intein-based system with a “modified intein” component.

The term “modified intein” as used herein can refer to a synthetic intein or a natural intein wherein at least one amino acid residue is substituted in, deleted from, or added to, the intein splicing unit so that the cleaved or excised exteins are not completely ligated by the intein.

The term “vector”, as used herein, refers to a DNA or RNA molecule such as a plasmid, virus or other vehicle, which contains one or more heterologous or recombinant DNA sequences and is designed for transfer between different host cells. The terms “expression vector” and “gene therapy vector” refer to any vector that is effective to incorporate and express heterologous DNA fragments in a cell. A cloning or expression vector may comprise additional elements, for example, the expression vector may have two replication systems, thus allowing it to be maintained in two organisms, for example in human cells for expression and in a prokaryotic host for cloning and amplification. Any suitable vector can be employed that is effective for introduction of nucleic acids into cells such that protein or polypeptide expression results, e.g. a viral vector or non-viral plasmid vector. Any cells effective for expression, e.g., insect cells and eukaryotic cells such as yeast or mammalian cells are useful in practicing the invention.

The terms “heterologous DNA” and “heterologous RNA” refer to nucleotides that are not endogenous (native) to the cell or part of the genome or vector in which they are present. Generally heterologous DNA or RNA is added to a cell by transduction, infection, transfection, transformation, electroporation, biolistic transformation or the like. Such nucleotides generally include at least one coding sequence, but the coding sequence need not be expressed. The term “heterologous DNA” may refer to a “heterologous coding sequence” or a “transgene”.

As used herein, the terms “protein” and “polypeptide” may be used interchangeably and typically refer to “proteins” and “polypeptides” of interest that are expressed using the self processing cleavage site-containing vectors of the present invention. Such “proteins” and “polypeptides” may be any protein or polypeptide useful for research, diagnostic or therapeutic purposes, as further described below. As used herein, a polyprotein is a protein which is destined for processing to produce two or more polypeptide products.

As used herein, the term “multimer” refers to a protein comprised of two or more polypeptide chains (sometimes refered to as “subunits”), which assemble to form a function protein. Multimers may be composed of two (dimers), three, (trimers), four (tetramers), or more (e.g., pentamers, and so on) peptide chains. Multimers may result from self-assembly, or may require a component such as a catalyst to assist in assembly. Multimers may be composed solely of identical peptide chains (homo-multimer), or two or more different peptide chains (hetero-multimers). Such multimers may structurally or chemically functional. Many multimers are known and used in the art, including but not limited to enzymes, hormones, antibodies, cytokines, chemokines, and receptors. As such, multimers can have both biological (e.g., pharmaceutical) and industrial (e.g., bioprocessing/bioproduction) utility.

As used herein, the term “tag” refers to a peptide, which may incorporated into an expression vector that that may function to allow detection and/or purification of one or more expression products of the vector inserts. Such tags are well-known in the art and may include a radiolabeled amino acid or attachment to a polypeptide of biotinyl moieties that can be detected by marked avidin (e.g., streptavidin containing a fluorescent marker or enzymatic activity that can be detected by optical or colorimetric methods). Affinity tags such as FLAG, glutathione-S-transferase, maltose binding protein, cellulose-binding domain, thioredoxin, NusA, mistin, chitin-binding domain, cutinase, AGT, GFP and others are widely used such as in protein expression and purification systems. Further nonlimiting examples of tags for polypeptides include, but are not limited to, the following: Histidine tag, radioisotopes or radionuclides (e.g., ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, or ¹⁵³Sm); fluorescent tags (e.g., FITC, rhodamine, lanthanide phosphors), enzymatic tags (e.g., horseradish peroxidase, luciferase, alkaline phosphatase); chemiluminescent tags; biotinyl groups; predetermined polypeptide epitopes recognized by a secondary reporter (e.g., leucine zipper pair sequences, binding sites for secondary antibodies, metal binding domains, epitope tags); and magnetic agents, such as gadolinium chelates.

The term “replication defective” as used herein relative to a viral gene therapy vector of the invention means the viral vector cannot independently further replicate and package its genome. For example, when a cell of a subject is infected with rAAV virions, the heterologous gene is expressed in the infected cells, however, due to the fact that the infected cells lack AAV rep and cap genes and accessory function genes, the rAAV is not able to replicate.

As used herein, a “retroviral transfer vector” refers to an expression vector that comprises a nucleotide sequence that encodes a transgene and further comprises nucleotide sequences necessary for packaging of the vector. Preferably, the retroviral transfer vector also comprises the necessary sequences for expressing the transgene in cells.

As used herein, “packaging system” refers to a set of viral constructs comprising genes that encode viral proteins involved in packaging a recombinant virus. Typically, the constructs of the packaging system are ultimately incorporated into a packaging cell.

As used herein, a “second generation” lentiviral vector system refers to a lentiviral packaging system that lacks functional accessory genes, such as one from which the accessory genes, vif, vpr, vpu and nef, have been deleted or inactivated. See, e.g., Zufferey et al. 1997. Nat. Biotechnol. 15:871-875.

As used herein, a “third generation” lentiviral vector system refers to a lentiviral packaging system that has the characteristics of a second generation vector system, and further lacks a functional that gene, such as one from which the that gene has been deleted or inactivated. Typically, the gene encoding rev is provided on a separate expression construct. See, e.g., Dull et al. 1998. J. Virol. 72:8463-8471.

As used herein with respect to a virus or viral vector, “pseudotyped” refers to the replacement of a native virus envelope protein with a heterologous or functionally modified virus envelope protein.

The term “operably linked” as used herein relative to a recombinant DNA construct or vector means nucleotide components of the recombinant DNA construct or vector are usually covalently joined to one another. Generally, “operably linked” DNA sequences are contiguous, and, in the case of a secretory leader, contiguous and in the same reading frame. However, enhancers do not have to be contiguous with the sequences whose expression is upregulated. The term is consistent with operably positioned.

Enhancer sequences influence promoter-dependent gene expression and may be located in the 5′ or 3′ regions of the native gene. “Enhancers” are cis-acting elements that stimulate or inhibit transcription of adjacent genes. An enhancer that inhibits transcription also is termed a “silencer”. Enhancers can function (i.e., can be associated with a coding sequence) in either orientation, over distances of up to several kilobase pairs (kb) from the coding sequence and from a position downstream of a transcribed region. In addition, insulator or chromatin opening sequences, such as matrix attachment regions (Chung, Cell, 1993, Aug. 13; 74(3):505-14, Frisch et al, Genome Research, 2001, 12:349-354, Kim et al, J. Biotech 107, 2004, 95-105) may be used to enhance transcription of stably integrated gene cassettes.

As used herein, the term “gene” or “coding sequence” means the nucleic acid sequence which is transcribed (DNA) and translated (mRNA) into a polypeptide in vitro or in vivo when operably linked to appropriate regulatory sequences. The gene may or may not include regions preceding and following the coding region, e.g. 5′ untranslated (5′ UTR) or “leader” sequences and 3′ UTR or “trailer” sequences, as well as intervening sequences (introns) between individual coding segments (exons).

A “promoter” is a DNA sequence that directs the binding of RNA polymerase and thereby promotes RNA synthesis, i.e., a minimal sequence sufficient to direct transcription. Promoters and corresponding protein or polypeptide expression may be cell-type specific, tissue-specific, or species specific. Also included in the nucleic acid constructs or vectors of the invention are enhancer sequences which may or may not be contiguous with the promoter sequence.

“Transcription regulatory sequences”, or expression control sequences, as broadly used herein, include a promoter sequence and physically associated sequences which modulate or regulate transcription of an associated coding sequence, often in response to nutritional or environmental signals. Those associated sequences can determine tissue or cell specific expression, response to an environmental signal, binding of a protein which increases or decreases transcription, and the like. A “regulatable promoter” is any promoter whose activity is affected by a cis or trans acting factor (e.g., an inducible promoter, which is activated by an external signal or agent).

A “constitutive promoter” is any promoter that directs RNA production in many or all tissue/cell types at most times, e.g., the human CMV immediate early enhancer/promoter region which promotes constitutive expression of cloned DNA inserts in mammalian cells.

The terms “transcriptional regulatory protein”, “transcriptional regulatory factor” and “transcription factor” are used interchangeably herein, and refer to a nuclear protein that binds a DNA response element and thereby transcriptionally regulates the expression of an associated gene or genes. Transcriptional regulatory proteins generally bind directly to a DNA response element, however in some cases binding to DNA may be indirect by way of binding to another protein that in turn binds to, or is bound to a DNA response element.

As used herein, the terms “immunoglobulin” and “antibody” refer to intact molecules as well as fragments thereof, such as Fa, F(ab′)2, and Fv, which are capable of binding an antigenic determinant of interest. Such an “immunoglobulin” and “antibody” is composed of two identical light polypeptide chains of molecular weight approximately 23,000 daltons, and two identical heavy chains of molecular weight 53,000-70,000. The four chains are joined by disulfide bonds in a “Y” configuration. Heavy chains are classified as gamma (IgG), mu (IgM), alpha (IgA), delta (IgD) or epsilon (IgE) and are the basis for the class designations of immunoglobulins, which determines the effector function of a given antibody. Light chains are classified-as either kappa or lambda. When reference is made herein to an “immunoglobulin or fragment thereof”, it will be understood that such a “fragment thereof” is an immunologically functional immunoglobulin fragment, especially one which binds its cognate ligand with binding affinity of at least 10% that of the intact immunoglobulin.

An Fab fragment of an antibody is a monovalent antigen-binding fragment of an antibody molecule. An Fv fragment is a genetically engineered fragment containing the variable region of a light chain and the variable regions of a heavy chain expressed as two chains.

The term “humanized antibody” refers to an antibody molecule in which one or more amino acids have been replaced in the non-antigen binding regions in order to more closely resemble a human antibody, while still retaining the original binding activity of the antibody. See, e.g., U.S. Pat. No. 6,602,503.

The term “antigenic determinant”, as used herein, refers to that fragment of a molecule (i.e., an epitope) that makes contact with a particular antibody. Numerous regions of a protein or peptide or glycopeptide of a protein or glycoprotein may induce the production of antibodies which bind specifically to a given region or three-dimensional structure on the protein. These regions or structures are referred to as antigenic determinants or epitopes. An antigenic determinant may compete with the intact antigen (i.e., the immunogen used to elicit the immune response) for binding to an antibody.

The term “fragment,” when referring to a recombinant protein or polypeptide of the invention means a peptide or polypeptide which has an amino acid sequence which is the same as part of, but not all of, the amino acid sequence of the corresponding full length protein or polypeptide, which retains at least one of the functions or activities of the corresponding full length protein or polypeptide. The fragment preferably includes at least 20-100 contiguous amino acid residues of the full length protein or polypeptide.

The terms “administering” or “introducing”, as used herein, mean delivering the protein (include immunoglobulin) to a human or animal in need thereof by any route known to the art. Pharmaceutical carriers and formulations or compositions are also well known to the art. Routes of administration can include intravenous, intramuscular, intradermal, subcutaneous, transdermal, mucosal, intratumoral or mucosal. Alternatively, these terms can refer to delivery of a vector for recombinant protein expression to a cell or to cells in culture and or to cells or organs of a subject. Such administering or introducing may take place in vivo, in vitro or ex vivo. A vector for recombinant protein or polypeptide expression may be introduced into a cell by transfection, which typically means insertion of heterologous DNA into a cell by physical means (e.g., calcium phosphate transfection, electroporation, microinjection or lipofection); infection, which typically refers to introduction by way of an infectious agent, i.e. a virus; or transduction, which typically means stable infection of a cell with a virus or the transfer of genetic material from one microorganism to another by way of a viral agent (e.g., a bacteriophage).

“Transformation” is typically used to refer to bacteria comprising heterologous DNA or cells which express an oncogene and have therefore been converted into a continuous growth mode, for example, tumor cells. A vector used to “transform” a cell may be a plasmid, virus or other vehicle.

Typically, a cell is referred to as “transduced”, “infected”, “transfected” or “transformed” dependent on the means used for administration, introduction or insertion of heterologous DNA (i.e., the vector) into the cell. The terms “transduced”, “transfected” and “transformed” may be used interchangeably herein regardless of the method of introduction of heterologous DNA.

As used herein, the terms “stably transformed”, “stably transfected” and “transgenic” refer to cells that have a non-native (heterologous) nucleic acid sequence integrated into the genome. Stable transfection is demonstrated by the establishment of cell lines or clones comprised of a population of daughter cells containing the transfected DNA stably replicating by means of integration into their genomes or as an episomal element. In some cases, “transfection” is not stable, i.e., it is transient. In the case of transient transfection, the exogenous or heterologous DNA is expressed, however, the introduced sequence is not integrated into the genome or the host cell is not able to replicate.

As used herein, “ex vivo administration” refers to a process where primary cells are taken from a subject, a vector is administered to the cells to produce transduced, infected or transfected recombinant cells and the recombinant cells are readministered to the same or a different subject.

A “multicistronic transcript” refers to an mRNA molecule that contains more than one protein coding region, or cistron. A mRNA comprising two coding regions is denoted a “bicistronic transcript.” The “5′-proximal” coding region or cistron is the coding region whose translation initiation codon (usually AUG) is closest to the 5′ end of a multicistronic mRNA molecule. A “5′-distal” coding region or cistron is one whose translation initiation codon (usually AUG) is not the closest initiation codon to the 5′ end of the mRNA.

The terms “5′-distal” and “downstream” are used synonymously to refer to coding regions that are not adjacent to the 5′ end of a mRNA molecule.

As used herein, “co-transcribed” means that two (or more) open reading frames or coding regions or polynucleotides are under transcriptional control of a single transcriptional control or regulatory element comprising a promoter.

The term “host cell”, as used herein refers to a cell which has been transduced, infected, transfected or transformed with a vector. The vector may be a plasmid, a viral particle, a phage, etc. The culture conditions, such as temperature, pH and the like, are those previously used with the host cell selected for expression, and will be apparent to those skilled in the art. It will be appreciated that the term “host cell” refers to the original transduced, infected, transfected or transformed cell and progeny thereof.

As used herein, the terms “biological activity” and “biologically active”, refer to the activity attributed to a particular protein in a cell line in culture or in a cell-free system, such as a ligand-receptor assay in ELISA plates. The “biological activity” of an “immunoglobulin”, “antibody” or fragment thereof refers to the ability to bind an antigenic determinant and thereby facilitate immunological function. The “biological activity” of a hormone or interleukin is as known in the art.

As used herein, the terms “tumor” and “cancer” refer to a cell that exhibits at least a partial loss of control over normal growth and/or development. For example, often tumor or cancer cells generally have lost contact inhibition and may be invasive and/or have the ability to metastasize.

Antibodies are immunoglobulin proteins that are heterodimers of a heavy and light chain. An typical antibody is multimeric with two heavy chains and two light chains (or functional fragments thereof) which associate together. Antibodies can have a further polymeric order of structure in being dimeric, trimeric, tetrameric, pentameric, etc., often dependent on isotype. They have proven extremely difficult to express in a full length form from a single vector or from two vectors in mammalian culture expression systems. Several methods are currently used for production of antibodies: in vivo immunization of animals to produce “polyclonal” antibodies, in vitro cell culture of B-cell hybridomas to produce monoclonal antibodies (Kohler, et al. 1988. Eur. J. Immunol. 6:511; Antibodies: A Laboratory Manual, Cold Spring Harbor Laboratory, 1988; incorporated by reference herein) and recombinant DNA technology (described for example in Cabilly et al., U.S. Pat. No. 6,331,415, incorporated by reference herein).

The basic molecular structure of immunoglobulin polypeptides is well known to include two identical light chains with a molecular weight of approximately 23,000 daltons, and two identical heavy chains with a molecular weight 53,000-70,000, where the four chains are joined by disulfide bonds in a “Y” configuration. The amino acid sequence runs from the N-terminal end at the top of the Y to the C-terminal end at the bottom of each chain. At the N-terminal end is a variable region (of approximately 100 amino acids in length) which provides for the specificity of antigen binding.

The present invention is directed to improved methods for production of immunoglobulins of all types, including, but not limited to, full length antibodies and antibody fragments having a native sequence (i.e. that sequence produced in response to stimulation by an antigen), single chain antibodies which combine the antigen binding variable region of both the heavy and light chains in a single stably-folded polypeptide chain; univalent antibodies (which comprise a heavy chain/light chain dimer bound to the Fc region of a second heavy chain); “Fab fragments” which include the full “Y” region of the immunoglobulin molecule, i.e., the branches of the “Y”, either the light chain or heavy chain alone, or portions, thereof (i.e., aggregates of one heavy and one light chain, commonly known as Fab′); “hybrid immunoglobulins” which have specificity for two or more different antigens (e.g., quadromas or bispecific antibodies as described for example in U.S. Pat. No. 6,623,940); “composite immunoglobulins” wherein the heavy and light chains mimic those from different species or specificities; and “chimeric antibodies” wherein portions of each of the amino acid sequences of the heavy and light chain are derived from more than one species (i.e., the variable region is derived from one source such as a murine antibody, while the constant region is derived from another, such as a human antibody).

The compositions and methods of the invention find utility in production of immunoglobulins or fragments thereof wherein the heavy or light chain is “mammalian”, “chimeric” or modified in a manner to enhance its efficacy. Modified antibodies include both amino acid and nucleic acid sequence variants which retain the same biological activity of the unmodified form and those which are modified such that the activity is altered, i.e., changes in the constant region that improve complement fixation, interaction with membranes, and other effector functions, or changes in the variable region that improve antigen binding characteristics. The compositions and methods of the invention can further include catalytic immunoglobulins or fragments thereof.

A “variant” immunoglobulin-encoding polynucleotide sequence may encode a “variant” immunoglobulin amino acid sequence which is altered by one or more amino acids from the reference polypeptide sequence. This same discussion which follows is applicable to other biologically active protein sequences (and their coding sequences) of interest. The variant polynucleotide sequence may encode a variant amino acid sequence which contains “conservative” substitutions, wherein the substituted amino acid has structural or chemical properties similar to the amino acid which it replaces. It is understood that a variant of a the protein of interest can be made with an amino acid sequence which is substantially identical (at least about 80 to 99% identical, and all integers there between) to the amino acid sequence of the naturally occurring sequence, and it forms a functionally equivalent, three dimensional structure and retains the biological activity of the naturally occurring protein. It is well known in the biological arts that certain amino acid substitutions can be made in protein sequences without affecting the function of the protein. Generally, conservative amino acid substitutions or substitutions of similar amino acids are tolerated without affecting protein function. Similar amino acids can be those that are similar in size and/or charge properties, for example, aspartate and glutamate and isoleucine and valine are both pairs of similar amino acids. Substitutions of one for another are permitted when native secondary and tertiary structure formation are not disrupted except as intended. Similarity between amino acid pairs has been assessed in the art in a number of ways. For example, Dayhoff et al. , in Atlas of Protein Sequence and Structure, 1978. Volume 5, Supplement 3, Chapter 22, pages 345-352, which is incorporated by reference herein, provides frequency tables for amino acid substitutions which can be employed as a measure of amino acid similarity. Dayhoff et al.'s frequency tables are based on comparisons of amino acid sequences for proteins having the same function from a variety of evolutionarily different sources.

Substitution mutation, insertional, and deletional variants of the disclosed nucleotide (and amino acid) sequences can be readily prepared by methods which are well known to the art. These variants can be used in the same manner as the specifically exemplified sequences so long as the variants have substantial sequence identity with a specifically exemplified sequence of the present invention and the desired functionality is preserved.

As used herein, substantial sequence identity refers to homology (or identity) which is sufficient to enable the variant polynucleotide or protein to function in the same capacity as the polynucleotide or protein from which the variant is derived. Preferably, this sequence identity is greater than 70% or 80%, more preferably, this identity is greater than 85%, or this identity is greater than 90%, and/or alternatively, this is greater than 95%, and all integers between 70 and 100%. It is well within the skill of a person trained in this art to make substitution mutation, insertional, and deletional mutations which are equivalent in function or are designed to improve the function of the sequence or otherwise provide a methodological advantage. No embodiments/variants which may read on any naturally occurring proteins or which read on a qualifying prior art item are intended to be within the scope of the present invention as claimed. It is well known in the art that the polynucleotide sequences of the present invention can be truncated and/or otherwise mutated such that certain of the resulting fragments and/or mutants of the original full-length sequence can retain the desired characteristics of the full-length sequence. A wide variety of restriction enzymes which are suitable for generating fragments from larger nucleic acid molecules are well known. In addition, it is well known that Ba/31 exonuclease can be conveniently used for time-controlled limited digestion of DNA. See, for example, Maniatis et al. 1982. Molecular Cloning: A Laboratory Manual, Cold Spring Harbor Laboratory, New York, pages 135-139, incorporated herein by reference. See also Wei et al. 1983. J. Biol. Chem. 258:13006-13512. By use of Ba/31 exonuclease (commonly referred to as “erase-a-base” procedures), the ordinarily skilled artisan can remove nucleotides from either or both ends of the subject nucleic acids to generate a wide spectrum of fragments which are functionally equivalent to the subject nucleotide sequences. One of ordinary skill in the art can, in this manner, generate hundreds of fragments of controlled, varying lengths from locations all along the original coding sequence. The ordinarily skilled artisan can routinely test or screen the generated fragments for their characteristics and determine the utility of the fragments as taught herein. It is also well known that the mutant sequences of the full length sequence, or fragments thereof, can be easily produced with site directed mutagenesis. See, for example, Larionov, O. A. and Nikiforov, V. G. 1982. Genetika 18:349-59; Shortle et al. (1981) Annu. Rev. Genet. 15:265-94; both incorporated herein by reference. The skilled artisan can routinely produce deletion-, insertion-, or substitution-type mutations and identify those resulting mutants which contain the desired characteristics of the full length wild-type sequence, or fragments thereof, e.g., those which retain hormone, cytokine, antigen-binding or other biological activity.

In addition, or alternatively, the variant polynucleotide sequence may encode a variant amino acid sequence which contains “non-conservative” substitutions, wherein the substituted amino acid has dissimilar structural or chemical properties to the amino acid which it replaces. Variant immunoglobulin-encoding polynucleotides may also encode variant amino acid sequences which contain amino acid insertions or deletions, or both. Furthermore, a variant “immunoglobulin-encoding polynucleotide may encode the same polypeptide as the reference polynucleotide sequence but, due to the degeneracy of the genetic code, has a polynucleotide sequence which is altered by one or more bases from the reference polynucleotide sequence.

The term “fragment,” when referring to a recombinant immunoglobulin of the invention means a polypeptide which has an amino acid sequence which is the same as part of but not all of the amino acid sequence of the corresponding full length immunoglobulin protein, which either retains essentially the same biological function or activity as the corresponding full length protein, or retains at least one of the functions or activities of the corresponding full length protein. The fragment preferably includes at least 20-100 contiguous amino acid residues of the full length immunoglobulin, and preferably, retains the ability to bind the same antigen as the full length antibody.

As used herein, the term “sequence identity” means nucleic acid or amino acid sequence identity in two or more aligned sequences, when aligned using a sequence alignment program. The term “% homology” is used interchangeably herein with the term “% identity” herein and refers to the level of nucleic acid or amino acid sequence identity between two or more aligned sequences, when aligned using a sequence alignment program. For example, as used herein, 80% homology means the same thing as 80% sequence identity determined by a defined algorithm as would be understood in the art, and accordingly a homologue of a given sequence has greater than 80% sequence identity over a length of the given sequence.

Optimal alignment of sequences for comparison can be conducted, e.g., by the local homology algorithm of Smith and Waterman. 1981. Adv. Appl. Math. 2:482, by the homology alignment algorithm of Needleman and Wunsch. 1970. J Mol. Biol. 48:443, by the search for similarity method of Pearson and Lipman. 1988. Proc. Natl. Acad. Sci. USA 85:2444, by computerized implementations of these algorithms (GAP, BESTFIT, FASTA, and TFASTA in the Wisconsin Genetics software Package, Genetics Computer Group, Madison, Wis.), by the BLAST algorithm, Altschul et al. 1990. J Mol. Biol. 215:403-410, with software that is publicly available through the National Center for Biotechnology Information website (see nlm.nih.gov/), or by visual inspection (see generally, Ausubel et al., infra). For purposes of the present invention, optimal alignment of sequences for comparison is most preferably conducted by the local homology algorithm of Smith and Waterman. 1981. Adv. Appl. Math. 2:482. See, also, Altschul et al. 1990 and Altschul et al. 1997.

The terms “identical” or percent “identity” in the context of two or more nucleic acid or protein sequences, refer to two or more sequences or subsequences that are the same or have a specified percentage of amino acid residues or nucleotides that are the same, when compared and aligned for maximum correspondence, as measured using one of the sequence comparison algorithms described herein, e.g. the Smith-Waterman algorithm, others known in the art, e.g., BLAST, or by visual inspection.

In accordance with the present invention, also encompassed are sequence variants which encode self-processing cleavage polypeptides and polypeptides themselves that have 80, 85, 88, 89, 90, 91, 92, 93, 94, 95, 96, 97, 98, 99% (and all integers between 80 and 100 for percentage values) or more sequence identity to the native or reference sequence. Also encompassed are amino acid fragments of the polypeptides that represent a continuous stretch of at least 5, at least 10, or at least 15 units; and fragments homologous thereto according to the described identity conditions; and fragments of nucleic acid sequences that represent a continuous stretch of at least 15, at least 30, or at least 45 units. In a particular embodiment, a nucleic acid sequence or amino acid sequence is 90%, 91%, 92%, 93%, 94%, 95%, 96%, 97%, 98%, 99%, or 99.5% identical to a respective sequence disclosed herewith.

A nucleic acid sequence is considered to be “selectively hybridizable” to a reference nucleic acid sequence if the two sequences specifically hybridize to one another under moderate to high stringency hybridization and wash conditions. Hybridization conditions are based on the melting temperature (Tm) of the nucleic acid binding complex or probe. For example, “maximum stringency” typically occurs at about Tm-5° C. (5° C. below the Tm of the probe); “high stringency” at about 5-10° C. below the Tm; “intermediate stringency” at about 10-20° C. below the Tm of the probe; and “low stringency” at about 20-25° C. below the Tm. Functionally, maximum stringency conditions may be used to identify sequences having strict identity or near-strict identity with the hybridization probe; while high stringency conditions are used to identify sequences having about 80% or more sequence identity with the probe.

Moderate and high stringency hybridization conditions are well known in the art (see, for example, Sambrook, et al, 1989, Chapters 9 and 11, and in Ausubel, F. M., et al., 1993. An example of high stringency conditions includes hybridization at about 42° C. in 50% formamide, 5×SSC, 5× Denhardt's solution, 0.5% SDS and 100 μg/ml denatured carrier DNA followed by washing two times in 2×SSC and 0.5% SDS at room temperature and two additional times in 0.1×SSC and 0.5% SDS at 42° C. 2A sequence variants that encode a polypeptide with the same biological activity as the naturally occurring protein of interest and hybridize under moderate to high stringency hybridization conditions are considered to be within the scope of the present invention.

As a result of the degeneracy of the genetic code, a number of coding sequences can be produced which encode the same polypeptide sequence, including such for structural components, self-processing components, e.g. inteins, regulatory components, e.g., signal peptidase cleavage sequences, or other components. For example, the triplet CGT encodes the amino acid arginine. Arginine is alternatively encoded by triplet nucleotide sequences CGA, CGC, CGG, AGA, and AGG. Therefore it is appreciated that such substitutions of synonymous codons in the coding region fall within the sequence variants that are covered by the present invention.

It is further appreciated that such sequence variants may or may not hybridize to the parent sequence under conditions of high stringency. This would be possible, for example, when the sequence variant includes a different codon for each of the amino acids encoded by the parent nucleotide. Such variants are, nonetheless, specifically contemplated and encompassed by the present invention.

The potential of antibodies as therapeutic modalities is currently limited by the production capacity and expense of the current technology. An improved viral or non-viral single expression vector for immunoglobulin (or other protein) production facilitates expression and delivery of two or more coding sequences, i.e., immunoglobulins or other proteins with bi- or multiple-specificities from a single vector. The present invention addresses these limitations and is applicable to any immunoglobulin (i.e. an antibody) or fragment thereof or other multipart protein or binding protein pair as further detailed herein, including engineered antibodies such as single chain antibodies, full-length antibodies or antibody fragments, two chain hormones, two chain cytokines, two chain chemokines, two chain receptors, and the like.

### Inteins

As used herein, an intein is a segment within an expressed protein, bounded toward the N-terminus of the primary expression product by an N-extein and bounded toward the C-terminus of the primary expression product by a C-extein. Naturally occurring inteins mediate excision of the inteins and rejoining (protein ligation) of the N- and C-exteins. However, in the context of the present expression products, the primary sequence of the intein or the flanking extein amino acid sequence is such that the cleavage of the protein backbone occurs in the absence of or with reduced or a minimal amount of ligation of the exteins, so that the extein proteins are released from the primary translation product (polyprotein) without their being joined to form a fusion protein. The intein portion of the primary expression product (the protein synthesized by mRNA, prior to any proteolytic cleavage) mediates the proteolytic cleavage at the N-extein/intein and the intein/C-extein junctions. In general, naturally occurring inteins also mediate the splicing together(joining by formation of a peptide bond) of the N-extein and the C-extein. However, in the present invention as applied to the goal of expressing two polypeptides (as specifically exemplified by the heavy and light chains of an antibody molecule), it is preferred that protein ligation does not occur. This can be achieved by incorporating an intein which either naturally or through mutation does not have ligation activity. Alternatively, splicing can be prevented by mutation to change the amino acid(s) at or next to the splice site to prevent ligation of the released proteins. See Xu and Perler, 1996, EMBO J. 15:5146-5153. For example, Ser, Thr or Cys normally occurs at the start of the C-extein and can be changed to modify or interrupt splicing. In a particular intein, the effect on splicing prevents or reduces the ligation of expressed proteins.

Inteins are a class of proteins whose genes are found only within the genes of other proteins. Together with the flanking host genes termed exteins, inteins are transcribed as a single mRNA, and translated as a single polypeptide. Post-translationally, inteins initiate an autocatalytic event to remove themselves and joint the flanking host protein segments with a new polypeptide bond. This reaction is catalyzed solely by the intein, require no other cellular proteins, co-factors, or ATP. Inteins are found in a variety of unicellular organisms and they have different sizes. Many inteins contain an endonuclease domain, which accounts for their mobility within genomes.

Intein mediated reactions have been used in biotechnology, especially for in vitro settings such as for purifications and for protein chip construction, and in plant strain improvement (Perler, F. B. 2005. IUBMB Life 57(7):469-76). Mutations have been introduced into native intein nucleotide sequences, and some of these mutants are reported to have altered properties (Xu and Perler, 1996. EMBO J. 15(9), 5146-5153). Besides inteins, bacterial intein-like (BIL) domains and hedgehog (Hog) auto-processing domains, the other 2 members of the Hog/intein (HINT) superfamily, are also know to catalyze post-translational self-processing through similar mechanisms (Dassa et.al. 2004. J. Biol. Chem. 279(31):32001-32007).

Inteins occur as in-frame insertions in specific host proteins. In a self-splicing reaction, inteins excise themselves from a precursor protein, while the flanking regions, the exteins, become joined to restore host gene function. These elements also contain an endonuclease function that accounts for their mobility within genomes. Inteins occur in a range of sizes (134 to 1650 amino acids), and they have been identified in the genomes of eubacteria, eukaryota and archaea. Experiments using model splicing/reporter systems have shown that the endonuclease, protein cleavage, and protein splicing functions can be separated (Xu and Perler. 1996. EMBO J. 15:5146-5153). The example described below uses an intein from Pyrococcus horikoshii Pho Pol I, Saccharomyces cerevisiae VMA, and Synechocystis spp. to create a fusion protein with sequences from an antibody heavy and light chain. Mutation of the intein designed to delete the intein's splicing capability results in a single polypeptide that undergoes a self-cleavage to produce correctly encoded antibody heavy and light chains. This strategy can be similarly employed in the expression of other multichain proteins, hormone or cytokines, and it can also be adapted for processing of precursor proteins (proproteins) to their mature, biologically active forms. While the use of the Pyrococcus horikoshii Pho Pol I, S. cerevisiae VMA, and Synechocystis spp. inteins are specifically exemplified herein, other inteins known to the art can be used in the polyprotein expression vectors and methods of the present invention.

Many other inteins besides the Pyrococcus horikoshii Pho Pol I, S. cerevisiae VMA, and Synechocystis spp. inteins are known to the art (See, e.g., Perler, F. B. 2002, InBase, the Intein Database, Nucl. Acids Res. 30(1):383-384 and the Intein Database and Registry, available via the New England Biolabs website, e.g., at http://tools.neb.com/inbase/). Inteins have been identified in a wide range of organisms such as yeast, mycobacteria and extreme thermophilic archaebacteria. Certain inteins have endonuclease activity as well as the site-specific protein cutting and splicing activities. Endonuclease activity is not necessary for the practice of the present invention; an endonuclease coding region can be deleted, provided that the protein cleavage activity is maintained.

The mechanism of the protein splicing process has been studied in great detail (Chong et al. 1996. J. Biol. Chem. 271: 22159-22168; Xu and Perler. 1996. EMBO J 15: 5146-5153), and conserved amino acids have been found at the intein and extein splicing points (Xu et al. 1994. EMBO J 13:5517-5522). Certain of the constructs described herein contain an intein sequence fused to the 3′-terminus of the first coding sequence, with a second coding sequence fused in frame at the C-terminus of the intein. Suitable intein sequences can be selected from any of the proteins known to contain protein splicing elements. A database containing all known inteins can be found on the World Wide Web (Perler, F. B. 1999. Nucl. Acids Res. 27: 346-347). The intein coding sequence is fused (in frame) at the 3′ end to the 5′ end of a second coding sequence. For targeting of this protein to a certain organelle, an appropriate peptide signal can be fused to the coding sequence of the protein.

After the second extein coding sequence, the intein coding sequence-extein coding sequence can be repeated as often as desired for expression of multiple proteins in the same cell. For multi-intein containing constructs, it may be useful to use intein elements from different sources. After the sequence of the last gene to be expressed, a transcription termination sequence, and advantageously including a polyadenylation sequence, is desirably inserted. The order of a polyadenylation sequence and a termination sequence can be as understood in the art. In an embodiment, a polyadenylation sequence can precede a termination sequence.

Modified intein splicing units have been designed so that such a modified intein of interest can catalyze excision of the exteins from the inteins but cannot catalyze ligation of the exteins (see, e.g., U.S. Pat. No. 7,026,526 and US Patent Publication 20020129400). Mutagenesis of the C-terminal extein junction in the Pyrococcus species GB-D DNA polymerase produced an altered splicing element that induces cleavage of exteins and inteins but prevents subsequent ligation of the exteins (Xu and Perler. 1996. EMBO J 15: 5146-5153). Mutation of serine 538 to either an alanine or glycine (Ser to Ala or Gly) induced cleavage but prevented ligation. At such position, Ser to Met or Ser to Thr are also used to achieve expression of a polyprotein that is cleaved into separate segments and at least partially not re-ligated. Mutation of equivalent residues in other intein splicing units can also prevent ligation of extein segments due to the relative conservation of amino acids at the C-terminal extein junction to the intein. In instances of low conservation/homology, for example, the first several, e.g., about five, residues of the C-extein and/or the last several residues of the intein segment are systematically varied and screened for the ability to support cleavage but not splicing of given extein segments, in particular extein segments disclosed herein and as understood in the art. There are inteins that do not contain an endonuclease domain; these include the Synechocystis spp dnaE intein and the Mycobacterium xenopi GyrA protein (Magnasco et al, Biochemistry, 2004, 43, 10265-10276; Telenti et al. 1997. J. Bacteriol. 179: 6378-6382). Others have been found in nature or have been created artificially by removing the endonuclease encoding domains from the sequences encoding endonuclease-containing inteins (Chong et al. 1997. J. Biol. Chem. 272: 15587-15590). Where desired, the intein is selected originally so that it consists of the minimal number of amino acids needed to perform the splicing function, such as the intein from the Mycobacterium xenopi GyrA protein (Telenti et al. 1997.supra). In an alternative embodiment, an intein without endonuclease activity is selected, such as the intein from the Mycobacterium xenopi GyrA protein or the Saccharomyces cerevisiae VMA intein that has been modified to remove endonuclease domains (Chong et al. 1997. supra).

Further modification of the intein splicing unit may allow the reaction rate of the cleavage reaction to be altered, allowing protein dosage to be controlled by simply modifying the gene sequence of the splicing unit.

In an embodiment, the first residue of the C-terminal extein is engineered to contain a glycine or alanine, a modification that was shown to prevent extein ligation with the Pyrococcus species GB-D DNA polymerase (Xu and Perler. 1996. EMBO J 15: 5146-5153). In this embodiment, preferred C-terminal extein proteins naturally contain a glycine or an alanine residue following the N-terminal methionine in the native amino acid sequence. Fusion of the glycine or alanine of the extein to the C-terminus of the intein provides the native amino acid sequence after processing of the polyprotein. In another embodiment, an artificial glycine or alanine is positioned in the C-terminal extein either by altering the native sequence or by adding an additional amino acid residue onto the N-terminus of the native sequence. In this embodiment, the native amino acid sequence of the protein will be altered by one amino acid after polyprotein processing. In further embodiments, other modifications useful in the present invention are described in U.S. Pat. No. 7,026,526.

In an embodiment, an intein is according to such in U.S. Pat. No. 7,026,526. In a particular embodiment, an intein is the Pyrococcus species GB-D DNA Polymerase intein. In an embodiment, mutation of the C-terminal extein serine to an alanine or glycine forms a modified intein splicing element that is capable of promoting excision of the polyprotein but not ligation of the extein units. In an embodiment, an intein is the Mycobacterium xenopi GyrA minimal intein of U.S. Pat. No. 7,026,526. In an embodiment, mutation of the C-terminal extein threonine to an alanine or glycine forms a modified intein splicing element that promotes excision of the polyprotein but does not ligate the extein units.

It will be appreciated that for certain inteins as described herein, embodiments of constructs and methods can generated improved expression of secreted protein product, particularly for multimeric proteins including antibodies.

### Signal Peptides and Signal Peptidases

The signal hypothesis, wherein proteins contain information within their amino acid sequences for protein targeting to the membrane, has been known for more than thirty years. Milstein and co-workers discovered that the light chain of IgG from myeloma cells was synthesized in a higher molecular weight form and was converted to its mature form when endoplasmic reticulum vesicles (microsomes) were added to the translation system, and proposed a model based on these results in which microsomes contain a protease that converts the precursor protein form to the mature form by removing the amino-terminal extension peptide. The signal hypothesis was soon expanded to include distinct targeting sequences within proteins localized to different intracellular membranes, such as the mitochondria and chloroplast. These distinct targeting sequences were later found to be cleaved from the exported protein by specific signal peptidases (SPases).

There are at least three distinct SPases involved in cleaving signal peptides in bacteria. SPase I can process nonlipoprotein substrates that are exported by the SecYEG pathway or the twin arginine translocation (Tat) pathway. Lipoproteins that are exported by the Sec pathway are cleaved by SPase II. SPase IV cleaves type IV prepilins and prepilin-like proteins that are components of the type II secretion apparatus.

In eukaryotes, proteins that are targeted to the endoplasmic reticulum (ER) membrane are mediated by signal peptides that target the protein either cotranslationally or post-translationally to the Sec61 translocation machinery. The ER signal peptides have features similar to those of their bacterial counterparts. The ER signal peptides are cleaved from the exported protein after export into the ER lumen by the signal peptidase complex (SPC).

The signal peptides that sort proteins to different locations within the eukaryotic cell have to be distinct because these cells contain many different membranous and aqueous compartments. Proteins that are targeted to the ER often contain cleavable signal sequences. Amazingly, many artificial peptides can function as translocation signals. The most important key feature is believed to be hydrophobicity above a certain threshold. ER signal peptides have a higher content of leucine residues than do bacterial signal peptides. The signal recognition particle (SRP) binds to cleavable signal peptides after they emerge from the ribosome. The SRP is required for targeting the nascent protein to the ER membrane. After translocation of the protein to the ER lumen, the exported protein is processed by the SPC. Another embodiment takes advantage of signal (leader) peptide processing enzymes which occur naturally in eukaryotic cells.

Most of known ER signal peptides are either N-terminal cleavable or internally uncleavable. Recently, a number of viral polyproteins such as those found in the hepatitis C virus, hantavirus, flavivirus, rubella virus, and influenza C virus were found to contain internal signal peptides that are most likely cleaved by the ER SPC. These studies on the maturation of polypropteins show that SPC can cleave not only amino-terminally located signal peptides, but also after internal signal peptides. Mutagenesis of the predicted signal peptidase substrate specificity elements may thus block viral infectivity.

The presenilin-type aspartic protease signal peptide peptidase (SPP) cleaves signal peptides within their transmembrane region. SPP is essential for generation of signal peptide-derived HLA-E epitopes in humans.

Signal peptidases are well known in the art. See, for example, Paetzel M. 2002. Chem. Rev. 102(12): 4549; Pekosz A. 1998. Proc. Natl. Acad. Sci. USA. 95:13233-13238; Marius K. 2002. Molecular Cell 10:735-744; Okamoto K. 2004. J. Virol. 78:6370-6380, Vol. 78; Martoglio B. 2003. Human Molecular Genetics 12: R201-R206; and Xia W. 2003. J. Cell Sci. 116:2839-2844.

Embodiments of this invention utilize internal cleavable signal peptides for expression of a polypeptide in a single transcript. The single transcribed polypeptide is then cleaved by SPC, leaving individual peptides separately or individual peptides being assembled into a protein. The methods of the present invention are applicable to the expression of immunoglobulin heavy chain and light chain in a single transcribed polypeptide, followed by cleavage, then assembly into a mature immunoglobulin. This technology is applicable to polypeptide cytokines, growth factors, or a variety of other proteins, for example, IL-12p40 and IL-12p35 in a single transcribed polypeptide and then assembly into IL-12, or IL-12p40 and IL-23p19 in a single transcribed polypeptide and then assembly into IL-23.

The signal peptidase approach is applicable to mammalian expression vectors which result in the expression of functional antibody or other processed product from a precursor or polyprotein. In the case of the antibody, it is produced from the vector as a polyprotein containing both heavy and light chains, with an intervening sequence between heavy chain and light chain being an internal cleavable signal peptide. This internal cleavable signal peptide can be cleaved by ER-residing proteases, mainly signal peptidases, presenilin or presenilin-like proteases, leaving heavy and light chains to fold and assemble to give a functional molecule, and desirably it is secreted. In addition to the internal cleavable signal peptide derived from hepatitis C virus, other internal cleavable sequences which can be cleaved by ER-residing proteases can be substituted thereof. Similarly, the practice of the invention need not be limited to host cells in which signal peptidase effects cleavage, but it also includes proteases including, but not limited to, presenilin, presenilin-like protease, and other proteases for processing polypeptides. Those proteases have been reviewed in the cited articles, among others.

In addition, the present invention is not limited to the expression of immunoglobulin heavy and light chains, but it also includes other polypeptides and polyproteins expressed in single transcripts followed by internal signal peptide cleavage to release each individual peptide or protein. These proteins may or may not assemble together in the mature product.

Also within the scope of the present invention are expression constructs in which the individual polypeptides are present in alternate orders, i.e., “Peptide 1-internal cleavable signal peptide- peptide 2” or “Peptide 2-internal cleavable signal peptide-peptide 1”. This invention further includes expression of more than two peptides linked by internal cleavable signal peptides, such as “Peptide 1-internal cleavable signal peptide- peptide 2- internal cleavable signal peptide- peptide 3”, and so on.

In addition, this invention applies to expression of both type I and type II transmembrane proteins and to other protease cleavage sites in connection with expression constructs.

This invention can further utilize internal cleavable signal peptides for maturation of one or more polypeptides within a polyprotein encoded within a single transcript. The single transcribed polypeptide is then cleaved by SPC, leaving individual peptides separately or individual peptides being assembled into a protein. Embodiments of this invention include compositions and methods to express immunoglobulin heavy chain and light chain in a single transcribed polypeptide with ultimate assembly into a mature immunoglobulin. This invention is applicable to express polypeptide cytokines, growth factors, or a variety of other proteins for example to express IL-12p40 and IL-12p35 in a single transcribed polypeptide and then assembly into IL-12, or IL-12p40 and IL-23p19 in a single transcribed polypeptide and then assembly into IL-23.

### Modification of Signal Peptide

In embodiments of sORF constructs, modified signal peptides are employed. For example, in a construct of Heavychain-int-LightChain, the antibody secretion level was increased about 10 fold when the hydrophobicity of the light chain signal peptide sequence was reduced through site-directed mutagenesis. Signal peptides can be employed as described in US 20070065912 by Carson et al., Mar. 22, 2007.

### Tags

Embodiments of sORF construct designs of the present invention include use of modified inteins that contain a tag, preferably an internal tag. A variety of tags are known in the art. Tags of the present invention include but are not limited to fluorescent tags and chemiluminescent tags. Using such constructs, the amount of polyprotein expressed can be monitored using fluorescent detection in individual cells. In addition, these cells can be sorted according to the level of protein expression using fluorescence activated cell sorting (FACS). The use of such tags are particularly useful in stable cell line generations as this allows the selection of high producing cells or cell lines through FACS analysis. As taught in the present invention, full length inteins have been observed in the cell lysate after their being auto-cleaved from the flanking antibody heavy and light chains. This provides bases for the detections of fluorescent labeled inteins and their use in stable cell line generation. Tags can also be used in purification of proteins.

### Mini-Inteins

Because endonuclease regions that are present in many inteins, including the P. horikoshii Poll intein and the Sce.VMA intein, are not particularly advantageous for gene expression systems, the endonuclease domain can optionally be deleted and replace with a small linker to create “mini-inteins”. These engineered mini-inteins are also useful in the described construct designs, and they present the advantage that the intein coding region is significantly smaller, thus allowing for a larger sequence encoding the polypeptides of interest and/or greater ease of handling the recombinant DNA molecules.

In embodiments it is advantageous to employ antibodies or analogues thereof with fully human characteristics. These reagents avoid the undesired immune responses induced by antibodies or analogues originating from non-human species. To address possible host immune responses to amino acid residues derived from self-processing peptides, the coding sequence for a proteolytic cleavage site may be inserted (using standard methodology known in the art) between the coding sequence for the first protein and the coding sequence for the self-processing peptide so as to remove the self-processing peptide sequence from the expressed polypeptide, i.e. the antibody. This finds particular utility in therapeutic or diagnostic antibodies for use in vivo.

### Gene Delivery and Vectors Including Viral Vectors

The present invention contemplates the use of any of a variety of vectors for introduction of constructs comprising the coding sequence for two or more polypeptides or proteins and a self processing cleavage sequence into cells. Numerous examples of gene expression vectors are known in the art and may be of viral or non-viral origin. Non-viral gene delivery methods which may be employed in the practice of the invention include but are not limited to plasmids, liposomes, nucleic acid/liposome complexes, cationic lipids and the like.

Viral and other vectors can efficiently transduce cells and introduce their own DNA into a host cell. In generating recombinant viral vectors, non-essential genes are replaced with expressible sequences encoding proteins or polypeptides of interest. Exemplary vectors include but are not limited to viral and non-viral vectors, such a retroviral vector (including lentiviral vectors), adenoviral (Ad) vectors including replication competent, replication deficient and gutless forms thereof, adeno-associated virus (AAV) vectors, simian virus 40 (SV-40) vectors, bovine papilloma vectors, Epstein-Barr vectors, herpes vectors, vaccinia vectors, Moloney murine leukemia vectors, Harvey murine sarcoma virus vectors, murine mammary tumor virus vectors, Rous sarcoma virus vectors and nonviral plasmids. Baculovirus vectors are well known and are suitable for expression in insect cells. A plethora of vectors suitable for expression in mammalian or other eukaryotic cells are well known to the art, and many are commercially available. Commercial sources include, without limitation, Stratagene, La Jolla, Calif.; Invitrogen, Carlsbad, Calif.; Promega, Madison, Wis. and Sigma-Aldrich, St. Louis, Mo. Many vector sequences are available through GenBank, and additional information concerning vectors is available on the internet via the Riken BioSource Center.

In an embodiment, the vector typically comprises an origin of replication and the vector may or may not in addition comprise a “marker” or “selectable marker” function by which the vector can be identified and selected. While any selectable marker can be used, selectable markers for use in recombinant vectors are generally known in the art and the choice of the proper selectable marker will depend on the host cell. Examples of selectable marker genes which encode proteins that confer resistance to antibiotics or other toxins include, but are not limited to ampicillin, methotrexate, tetracycline, neomycin (Southern et al. 1982. J Mol Appl Genet. 1:327-41), mycophenolic acid (Mulligan et al. 1980. Science 209:1422-7), puromycin, zeomycin, hygromycin (Sugden et al. 1985. Mol Cell Biol. 5:410-3), dihydrofolate reductase, glutamine synthetase, and G418. As will be understood by those of skill in the art, expression vectors typically include an origin of replication, a promoter operably linked to the coding sequence or sequences to be expressed, as well as ribosome binding sites, RNA splice sites, a polyadenylation site, and transcriptional terminator sequences, as appropriate to the coding sequence(s) being expressed.

Reference to a vector or other DNA sequences as “recombinant” merely acknowledges the operable linkage of DNA sequences which are not typically operably linked as isolated from or found in nature. Regulatory (expression and/or control) sequences are operatively linked to a nucleic acid coding sequence when the expression and/or control sequences regulate the transcription and, as appropriate, translation of the nucleic acid sequence. Thus expression and/or control sequences can include promoters, enhancers, transcription terminators, a start codon (i.e., ATG) 5′ to the coding sequence, splicing signals for introns and stop codons.

Adenovirus gene therapy vectors are known to exhibit strong transient expression, excellent titer, and the ability to transduce dividing and non-dividing cells in vivo (Hitt et al. 2000. Adv in Virus Res 55:479-505). Recombinant Ad vectors can comprise a packaging site enabling the vector to be incorporated into replication-defective Ad virions; the coding sequence for two or more polypeptides or proteins of interest, e.g., heavy and light chains of an immunoglobulin of interest; and a sequence encoding a self-processing cleavage site alone or in combination with an additional proteolytic cleavage site. Other elements necessary or helpful for incorporation into infectious virions, include the 5′ and 3′ Ad ITRs, the E2 genes, portions of the E4 gene and optionally the E3 gene.

Replication-defective Ad virions encapsulating the recombinant Ad vectors are made by standard techniques known in the art using Ad packaging cells and packaging technology. Examples of these methods may be found, for example, in U.S. Pat. No. 5,872,005. The coding sequence for two or more polypeptides or proteins of interest is commonly inserted into adenovirus in the deleted E3 region of the virus genome. Preferred adenoviral vectors for use in practicing the invention do not express one or more wild-type Ad gene products, e.g., E1a, E1b, E2, E3, and E4. Preferred embodiments are virions that are typically used together with packaging cell lines that complement the functions of E1, E2A, E4 and optionally the E3 gene regions. See, e.g. U.S. Pat. Nos. 5,872,005, 5,994,106, 6,133,028 and 6,127,175.

As used herein, “adenovirus” and “adenovirus particle” refer to the virus itself or derivatives thereof and cover all serotypes and subtypes and both naturally occurring and recombinant forms, except where indicated otherwise. Such adenoviruses may be wild type or may be modified in various ways known in the art or as disclosed herein. Such modifications include modifications to the adenovirus genome that is packaged in the particle in order to make an infectious virus. Such modifications include deletions known in the art, such as deletions in one or more of the E1a, E1b, E2a, E2b, E3, or E4 coding regions. Exemplary packaging and producer cells are derived from 293, A549 or HeLa cells. Adenovirus vectors are purified and formulated using standard techniques known in the art.

Adeno-associated virus (AAV) is a helper-dependent human parvovirus which is able to infect cells latently by chromosomal integration. Because of its ability to integrate chromosomally and its nonpathogenic nature, AAV has significant potential as a human gene therapy vector. For use in practicing the present invention rAAV virions may be produced using standard methodology, known to those of skill in the art and are constructed such that they include, as operatively linked components in the direction of transcription, control sequences including transcriptional initiation and termination sequences, and the coding sequence(s) of interest. More specifically, the recombinant AAV vectors of the instant invention comprise a packaging site enabling the vector to be incorporated into replication-defective AAV virions; the coding sequence for two or more polypeptides or proteins of interest, e.g., heavy and light chains of an immunoglobulin of interest; a sequence encoding a self-processing cleavage site alone or in combination with one or more additional proteolytic cleavage sites. AAV vectors for use in practicing the invention are constructed such that they also include, as operatively linked components in the direction of transcription, control sequences including transcriptional initiation and termination sequences. These components are flanked on the 5′ and 3′ end by functional AAV ITR sequences. By “functional AAV ITR sequences” is meant that the ITR sequences function as intended for the rescue, replication and packaging of the AAV virion.

Recombinant AAV vectors are also characterized in that they are capable of directing the expression and production of selected recombinant polypeptide or protein products in target cells. Thus, the recombinant vectors comprise at least all of the sequences of AAV essential for encapsidation and the physical structures for infection of the recombinant AAV (rAAV) virions. Hence, AAV ITRs for use in expression vectors need not have a wild-type nucleotide sequence (e.g., as described in Kotin. 1994. Hum. Gene Ther. 5:793-801), and may be altered by the insertion, deletion or substitution of nucleotides or the AAV ITRs may be derived from any of several AAV serotypes. Generally, an AAV vector can be any vector derived from an adeno-associated virus serotype known to the art.

Typically, an AAV expression vector is introduced into a producer cell, followed by introduction of an AAV helper construct, where the helper construct includes AAV coding regions capable of being expressed in the producer cell and which complement AAV helper functions absent in the AAV vector. The helper construct may be designed to down regulate the expression of the large Rep proteins (Rep78 and Rep68), typically by mutating the start codon following p5 from ATG to ACG, as described in U.S. Pat. No. 6,548,286, incorporated by reference herein. This is followed by introduction of helper virus and/or additional vectors into the producer cell, wherein the helper virus and/or additional vectors provide accessory functions capable of supporting efficient rAAV virus production. The producer cells are then cultured to produce rAAV. These steps are carried out using standard methodology. Replication-defective AAV virions encapsulating the recombinant AAV vectors of the instant invention are made by standard techniques known in the art using AAV packaging cells and packaging technology. Examples of these methods may be found, for example, in U.S. Pat. Nos. 5,436,146; 5,753,500, 6,040,183, 6,093,570 and 6,548,286, incorporated by reference herein in their entireties. Further compositions and methods for packaging are described in Wang et al. (US Patent Publication 2002/0168342), also incorporated by reference herein in its entirety, and include those techniques within the knowledge of those of skill in the art.

In practicing the invention, host cells for producing rAAV or other vector expression vector virions include mammalian cells, insect cells, microorganisms and yeast. Host cells can also be packaging cells in which the AAV (or other) rep and cap genes are stably maintained in the host cell or producer cells in which the AAV vector genome is stably maintained and packaged. Exemplary packaging and producer cells are derived from 293, A549 or HeLa cells. AAV vectors are purified and formulated using standard techniques known in the art. Additional suitable host cells (depending on the vector) include Chinese Hamster Ovary (CHO) cells, CHO dihydrofolate reductase deficient variants such as CHO DX B11 or CHO DG44 cells (see, e.g., Urlaub and Chasin. 1980. Proc. Natl. Acad. Sci. 77:4216-4220), PerC.6 cells (Jones et al. 2003. Biotechnol. Prog. 19:163-168) or Sp/20 mouse myeloma cells (Coney et al. 1994. Cancer Res. 54:2448-2455).

### Retroviral Vectors

Retroviral vectors can be used for gene delivery (Miller. 1992. Nature 357: 455-460). Retroviral vectors and more particularly lentiviral vectors may be used in practicing the present invention. Accordingly, the term “retrovirus” or “retroviral vector”, as used herein is meant to include “lentivirus” and “lentiviral vectors” respectively. Retroviral vectors have been tested and found to be suitable delivery vehicles for the stable introduction of genes of interest into the genome of a broad range of target cells. The ability of retroviral vectors to deliver unrearranged, single copy transgenes into cells makes retroviral vectors well suited for transferring genes into cells. Further, retroviruses enter host cells by the binding of retroviral envelope glycoproteins to specific cell surface receptors on the host cells. Consequently, pseudotyped retroviral vectors in which the encoded native envelope protein is replaced by a heterologous envelope protein that has a different cellular specificity than the native envelope protein (e.g., binds to a different cell-surface receptor as compared to the native envelope protein) may also find utility in practicing the present invention. The ability to direct the delivery of retroviral vectors encoding one or more target protein coding sequences to specific target cells is desirable in practice of the present invention.

The present invention provides retroviral vectors which include e.g., retroviral transfer vectors comprising one or more transgene sequences and retroviral packaging vectors comprising one or more packaging elements. In particular, the present invention provides pseudotyped retroviral vectors encoding a heterologous or functionally modified envelope protein for producing pseudotyped retrovirus.

The core sequence of the retroviral vectors of the present invention may be readily derived from a wide variety of retroviruses, including for example, B, C, and D type retroviruses as well as spumaviruses and lentiviruses (see RNA Tumor Viruses, Second Edition, Cold Spring Harbor Laboratory, 1985). An example of a retrovirus suitable for use in the compositions and methods of the present invention includes, but is not limited to, lentivirus. Other retroviruses suitable for use in the compositions and methods of the present invention include, but are not limited to, Avian Leukosis Virus, Bovine Leukemia Virus, Murine Leukemia Virus, Mink-Cell Focus-Inducing Virus, Murine Sarcoma Virus, Reticuloendotheliosis virus and Rous Sarcoma Virus. Particularly preferred Murine Leukemia Viruses include 4070A and 1504A (Hartley and Rowe. 1976. J. Virol. 19:19-25), Abelson (ATCC No. VR-999), Friend (ATCC No. VR-245), Graffi, Gross (ATCC No. VR-590), Kirsteni Harvey Sarcoma Virus and Rauscher (ATCC No. VR-998), and Moloney Murine Leukemia Virus (ATCC No. VR-190). Such retroviruses may be readily obtained from depositories or collections such as the American Type Culture Collection (ATCC; Manassas, Va.), or isolated from known sources using commonly available techniques. Others are available commercially.

In an embodiment, a retroviral vector sequence of the present invention can be derived from a lentivirus. A preferred lentivirus is a human immunodeficiency virus, e.g., type 1 or 2 (i.e., HIV-1 or HIV-2, wherein HIV-1 was formerly called lymphadenopathy associated virus 3 (HTLV-III) and acquired immune deficiency syndrome (AIDS)-related virus (ARV)), or another virus related to HIV-1 or HIV-2 that has been identified and associated with AIDS or AIDS-like disease. Other lentivirus include, a sheep Visna/maedi virus, a feline immunodeficiency virus (FIV), a bovine lentivirus, simian immunodeficiency virus (SIV), an equine infectious anemia virus (EIAV), and a caprine arthritis-encephalitis virus (CAEV).

Suitable genera and strains of retroviruses are well known in the art (see, e.g., Fields Virology, Third Edition, edited by B. N. Fields et al. 1996. Lippincott-Raven

Publishers, see e.g., Chapter 58, Retroviridae: The Viruses and Their Replication, Classification, pages 1768-1771, including Table 1 therein, incorporated herein by reference). Retroviral packaging systems for generating producer cells and producer cell lines that produce retroviruses, and methods of making such packaging systems are also known in the art.

Typical packaging systems comprise at least two packaging vectors: a first packaging vector which comprises a first nucleotide sequence comprising a gag, a pol, or gag and pol genes; and a second packaging vector which comprises a second nucleotide sequence comprising a heterologous or functionally modified envelope gene. The retroviral elements can be derived from a lentivirus, such as HIV. The vectors can lack a functional that gene and/or functional accessory genes (vif, vpr, vpu, vpx, nef). The system can further comprise a third packaging vector with a nucleotide sequence comprising a rev gene. The packaging system can be provided in the form of a packaging cell that contains the first, second, and, optionally, third nucleotide sequences.

In embodiments, there is applicability to a variety of expression systems, especially those with eukaryotic cells, and advantageously mammalian cells. Where native proteins are glycosylated, preferable embodiments can involve an expression system which will provide native-like glycosylation to the expressed proteins.

Lentiviruses share several structural virion proteins in common, including the envelope glycoproteins SU (gp120) and TM (gp41), which are encoded by the env gene; CA (p24), MA (p17) and NC (p7-11), which are encoded by the gag gene; and RT, PR and IN encoded by the pol gene. HIV-1 and HIV-2 contain accessory and other proteins involved in regulation of synthesis and processing virus RNA and other replicative functions. The accessory proteins, encoded by the vif, vpr, vpu/vpx, and nef genes, can be omitted (or inactivated) from the recombinant system. In addition, that and rev can be omitted or inactivated, e.g., by mutation or deletion.

First generation lentiviral vector packaging systems provide separate packaging constructs for gag/pol and env, and typically employ a heterologous or functionally modified envelope protein for safety reasons. In second generation lentiviral vector systems, the accessory genes, vif, vpr, vpu and nef, are deleted or inactivated. Third generation lentiviral vector systems are those from which the that gene has been deleted or otherwise inactivated (e.g., via mutation).

Compensation for the regulation of transcription normally provided by that can be provided by the use of a strong constitutive promoter, such as the human cytomegalovirus immediate early (HCAAV-IE) enhancer/promoter. Other promoters/enhancers can be selected based on strength of constitutive promoter activity, specificity for target tissue (e.g., a liver-specific promoter), or other factors relating to desired control over expression, as is understood in the art. For example, in some embodiments, it is desirable to employ an inducible promoter such as tet to achieve controlled expression. The gene encoding rev can be provided on a separate expression construct, such that a typical third generation lentiviral vector system will involve four plasmids: one each for gagpol, rev, envelope and the transfer vector. Regardless of the generation of packaging system employed, gag and pol can be provided on a single construct or on separate constructs.

Typically, the packaging vectors are included in a packaging cell, and are introduced into the cell via transfection, transduction or infection. Methods for transfection, transduction or infection are well known by those of skill in the art. A retroviral transfer vector of the present invention can be introduced into a packaging cell line, via transfection, transduction or infection, to generate a producer cell or cell line. The packaging vectors of the present invention can be introduced into human cells or cell lines by standard methods including, e.g., calcium phosphate transfection, lipofection or electroporation. In some embodiments, the packaging vectors are introduced into the cells together with a dominant selectable marker, such as neo, dihydrofolate reductase (DHFR), glutamine synthetase or ADA, followed by selection in the presence of the appropriate drug and isolation of clones. A selectable marker gene can be linked physically to genes encoded by the packaging vector.

Stable cell lines, wherein the packaging functions are configured to be expressed by a suitable packaging cell, are known. For example, see U.S. Pat. No. 5,686,279; and Ory et al. 1996. Proc. Natl. Acad. Sci. 93:11400-11406, which describe packaging cells. Further description of stable cell line production can be found in Dull et al. 1998. J. Virol. 72(11):8463-8471; and in Zufferey et al. 1998. J. Virol. 72:9873-9880.

Zufferey et al. 1997. Nat. Biotechnol. 15:871-75, teach a lentiviral packaging plasmid wherein sequences 3′ of pol including the HIV-1 envelope gene are deleted. The construct contains tat and rev sequences and the 3′ LTR is replaced with poly A sequences. The 5′ LTR and psi sequences are replaced by another promoter, such as one which is inducible. For example, a CMV promoter or derivative thereof can be used.

The packaging vectors may contain additional changes to the packaging functions to enhance lentiviral protein expression and to enhance safety. For example, all of the HIV sequences upstream of gag can be removed. Also, sequences downstream of the envelope can be removed. Moreover, steps can be taken to modify the vector to enhance the splicing and translation of the RNA.

Optionally, a conditional packaging system is used, such as that described by Dull et al. 1998. supra. Also preferred is the use of a self-inactivating vector (SIN), which improves the biosafety of the vector by deletion of the HIV-1 long terminal repeat (LTR) as described, for example, by Zufferey et al. 1998. J. Virol. 72:9873-9880. Inducible vectors can also be used, such as through a tetracycline-inducible LTR.

### Promoters

In embodiments, the vectors of the invention typically include heterologous control sequences, which include, but are not limited to, constitutive promoters, such as the cytomegalovirus (CMV) immediate early promoter, the RSV LTR, the MOMLV LTR, and the PGK promoter; tissue or cell type specific promoters including mTTR, TK, HBV, hAAT, regulatable or inducible promoters, enhancers, etc.

Certain useful promoters include the LSP promoter (III et al. 1997. Blood Coagul. Fibrinolysis 8S2:23-30), the EF1-alpha promoter (Kim et al. 1990. Gene 91(2):217-23) and Guo et al. 1996. Gene Ther. 3(9):802-10). Most preferred promoters include the elongation factor 1-alpha (EF1a) promoter, a phosphoglycerate kinase-1 (PGK) promoter, a cytomegalovirus immediate early gene (CMV) promoter, chimeric liver-specific promoters (LSPs), a cytomegalovirus enhancer/chicken beta-actin (CAG) promoter, a tetracycline responsive promoter (TRE), a transthyretin promoter (TTR), an simian virus 40 (SV40) promoter and a CK6 promoter. An advantageous promoter useful in the practice of the present invention is the adenovirus major late promoter (Berkner and Sharp. 1985. Nucl. Acids Res. 13:841-857). The structural and functional information of relevant promoters are known in the art. The relevant sequences may be readily obtained from public databases and incorporated into vectors for use in practicing aspects of the present invention.

A particularly preferred promoter in the practice of the present invention is the Adenovirus major late promoter. An expression cassette can comprise, in the 5′ to 3′ direction, an adenovirus major late promoter, a tripartite leader sequence operably to a first coding sequence for a protein of interest or protein chain of interest, a sequence encoding a self processing sequence or protease cleavage sequence, a second coding sequence for a protein or protein chain of interest, and optionally a sequence encoding a self processing sequence or protease cleavage sequence, followed by a third coding sequence for a protein or protein chain of interest. All of these coding sequences are covalently joined and in the same reading frame such that translation is not terminated within the polyprotein coding sequence. During protein synthesis or after completion of the synthesis of the polypeptide self processing or proteolytic processing cleaves the polyprotein into the appropriate protein chains or proteins. In the case of immunoglobulin synthesis, the coding sequence for light chain is present twice within the polyprotein coding sequence. Advantageously, leader sequence coding regions can be associated with the protein or protein chain sequences; processing by signal peptidases can have the added benefit of removing certain residual amino acid residues at the N-termini of proteins downstream of processing sites. Components for immunoglobulin heavy chain are Met, protein initiation methionine; HC, heavy chain; LC, light chain, SPPC, self-processing or protease cleavage site. Expression constructs for immunoglobulin synthesis can include the following: Met-protease-SPPC- HC leader sequence-HC-SPPC-LC leader sequence-LC-SPPC-LC leader sequence-LC; Met-protease-SPPC- LC leader sequence-LC-SPPC-LC leader sequence-LC-SPPC-HC leader sequence-HC; Met-protease-SPPC- LC leader sequence-LC-SPPC-HC leader sequence-HC-SPPC-LC leader sequence-LC; HC leader sequence-HC-SPPC-LC leader sequence-LC-SPPC-LC leader sequence-LC; LC leader sequence-LC-SPPC-HC leader sequence-HC-SPPC--LC leader sequence-LC; LC leader sequence-LC-SPPC-LC leader sequence-LC-SPPC-HC leader sequence-HC; Met-protease-SPPC-HC leader-HC-SPPC-LC leader-LC.

### Biotherapeutic Molecules Including Antibodies

Within the scope of the present invention, particular expressed antibodies (immunoglobulins) can include, inter alia, those which specifically bind tumor necrosis factor (engineered antibody corresponding to and/or derived from HUMIRA/D2E7; trademark for adalimumab of Abbott Biotechnology Ltd., Hamilton, Bermuda); interleukin-12 (engineered antibody derived from ABT-874); interleukin-18 (engineered antibody derived from ABT-325); recombinant erythropoietin receptor (engineered antibody derived from ABT-007); or E/L selectin (engineered antibody derived from EL246-GG). Coding and amino acid sequences of the engineered polyproteins are disclosed herewith or available in the art. Further antibodies which are suitable to the present invention include, e.g., Remicade (infliximab); Rituxan/Mabthera (rituximab); Herceptin (trastuzumab); Avastin (bevacizumab);Synagis (palivizumab); Erbitux (cetuximab); Reopro (abciximab); Orthoclone OKT3 (muromonab-CD3); Zenapax (daclizumab); Simulect (basiliximab); Mylotarg (gemtuzumab); Campath (alemtuzumab); Zevalin (ibritumomab); Xolair (omalizumab); Bexxar (tositumomab); and Raptiva (efalizumab); wherein generally a trademark-brand name is followed by a respective generic name in parentheses. Additional suitable proteins include, e.g., one or more of epoetin alfa, epoetin beta, etanercept, darbepoetin alfa, filgrastim, interferon beta 1a, interferon beta 1b, interferon alfa-2b, insulin glargine, somatropin, teriparatide, follitropin alfa, dornase, Factor VIII, Factor VII, Factor IX, imiglucerase, nesiritide, lenograstim, and Von Willebrand factor; wherein one or more generic designations may each correspond to one or more trademark-brand names of products. Other antibodies and proteins are suitable to the present invention as would be understood in the art.

The present invention also contemplates the controlled expression of the coding sequence for two or more polypeptides or proteins or proproteins of interest. Gene regulation systems are useful in the modulated expression of a particular gene or genes. In one exemplary approach, a gene regulation system or switch includes a chimeric transcription factor that has a ligand binding domain, a transcriptional activation domain and a DNA binding domain. The domains may be obtained from virtually any source and may be combined in any of a number of ways to obtain a novel protein. A regulatable gene system also includes a DNA response element which interacts with the chimeric transcription factor. This transcription regulatory element is located adjacent to the gene to be regulated.

Exemplary transcription regulation systems that may be employed in practicing the present invention include, for example, the Drosophila ecdysone system (Yao et al. 1996. Proc. Natl. Acad. Sci. 93:3346), the Bombyx ecdysone system (Suhr et al. 1998. Proc. Natl. Acad. Sci. 95:7999), the GeneSwitch (trademark of Valentis, The Woodlands, Tex.) synthetic progesterone receptor system which employs RU486 as the inducer (Osterwalder et al. 2001. Proc. Natl. Acad. Sci. USA 98(22):12596-601); the Tet and RevTet Systems (tetracycline regulated expression systems, trademarks of BD Biosciences Clontech, Mountain View, Calif.), which employ small molecules, such as tetracycline (Tc) or analogues, e.g. doxycycline, to regulate (turn on or off) transcription of the target (Knott et al. 2002. Biotechniques 32(4):796, 798, 800); ARIAD Regulation Technology (Ariad, Cambridge, Mass.) which is based on the use of a small molecule to bring together two intracellular molecules, each of which is linked to either a transcriptional activator or a DNA binding protein. When these components come together, transcription of the gene of interest is activated. Ariad has a system based on homodimerization and a system based on heterodimerization (Rivera et al. 1996. Nature Med. 2(9):1028-1032; Ye et al. 2000. Science 283:88-91).

Embodiments of the expression vector constructs of the invention comprising nucleic acid sequences encoding antibodies or fragments thereof or other heterologous proteins or pro-proteins in the form of self-processing or protease-cleaved recombinant polypeptides may be introduced into cells in vitro, ex vivo or in vivo for delivery of foreign, therapeutic or transgenes to cells, e.g., somatic cells, or in the production of recombinant polypeptides by vector-transduced cells.

### Host Cells and Delivery of Vectors

The vector constructs of the present invention may be introduced into suitable cells in vitro or ex vivo using standard methodology known in the art. Such techniques include, e.g., transfection using calcium phosphate, microinjection into cultured cells (Capecchi. 1980. Cell 22:479-488), electroporation (Shigekawa et al. 1988. BioTechnology 6:742-751), liposome-mediated gene transfer (Mannino et al. 1988. BioTechnology 6:682-690), lipid-mediated transduction (Feigner et al. 1987. Proc. Natl. Acad. Sci. USA 84:7413-7417), and nucleic acid delivery using high-velocity microprojectiles (Klein et al. 1987. Nature 327:70-73).

For in vitro or ex vivo expression, any cell effective to express a functional protein product may be employed. Numerous examples of cells and cell lines used for protein expression are known in the art. For example, prokaryotic cells and insect cells may be used for expression. In addition, eukaryotic microorganisms, such as yeast may be used. The expression of recombinant proteins in prokaryotic, insect and yeast systems are generally known in the art and may be adapted for antibody or other protein expression using the compositions and methods of the present invention.

Examples of cells useful for expression further include mammalian cells, such as fibroblast cells, cells from non-human mammals such as ovine, porcine, murine and bovine cells, insect cells and the like. Specific examples of mammalian cells include, without limitation, COS cells, VERO cells, HeLa cells, Chinese hamster ovary (CHO) cells, CHO DX B11 cells, CHO DG44 cells, PerC.6 cells, Sp2/0 cells, 293 cells, NSO cells, 3T3 fibroblast cells, W138 cells, BHK cells, HEPG2 cells, and MDCK cells.

Host cells are cultured in conventional nutrient media, modified as appropriate for inducing promoters, selecting transformants, or amplifying the genes encoding the desired sequences. Mammalian host cells may be cultured in a variety of media. Commercially available media such as Ham's F10 (Sigma), Minimal Essential Medium (MEM) (Sigma), RPMI 1640 (Sigma), Minimum Essential Medium (MEM) Alpha Medium, and Dulbecco's Modified Eagle's Medium (DMEM) (Sigma), are typically suitable for culturing host cells. A given medium is generally supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics, trace elements, and glucose or an equivalent energy source. Any other necessary supplements may also be included at appropriate concentrations as well known to those skilled in the art. The appropriate culture conditions for a particular cell line, such as temperature, pH and the like, are generally known in the art, with suggested culture conditions for culture of numerous cell lines, for example, in the ATCC Catalogue (available on the internet at “atcc.org/SearchCatalogs/AllCollections.cfm” or as instructed by commercial suppliers.

The expression vectors may be administered in vivo via various routes (e.g., intradermally, intravenously, intratumorally, into the brain, intraportally, intraperitoneally, intramuscularly, into the bladder etc.), to deliver multiple genes connected via a self processing cleavage sequence to express two or more proteins or polypeptides in animal models or human subjects. Dependent upon the route of administration, the therapeutic proteins elicit their effect locally (in brain or bladder) or systemically (other routes of administration). The use of tissue specific promoters 5′ to the open reading frame(s) results in tissue specific expression of the proteins or polypeptides encoded by the entire open reading frame.

Various methods that introduce a recombinant expression vector carrying a transgene into target cells in vitro, ex vivo or in vivo have been previously described and are well known in the art. The present invention provides for therapeutic methods, vaccines, and cancer therapies by infecting targeted cells with the recombinant vectors containing the coding sequence for two or more proteins or polypeptides of interest, and expressing the proteins or polypeptides in the targeted cell.

For example, in vivo delivery of the recombinant vectors of the invention may be targeted to a wide variety of organ types including, but not limited to brain, liver, blood vessels, muscle, heart, lung and skin.

In the case of ex vivo gene transfer, the target cells are removed from the host and genetically modified in the laboratory using recombinant vectors of the present invention and methods well known in the art.

The recombinant vectors of the invention can be administered using conventional modes of administration including but not limited to the modes described above. The recombinant vectors of the invention may be in a variety of formulations which include but are not limited to liquid solutions and suspensions, microvesicles, liposomes and injectable or infusible solutions. The preferred form depends upon the mode of administration and the therapeutic application.

In embodiments, advantages of the expression vector constructs in immunoglobulin or other biologically active protein production in vivo include administration of a single vector for long-term and sustained antibody expression in patients; in vivo expression of an antibody or fragment thereof (or other biologically active protein) having full biological activities; and the natural posttranslational modifications of the antibody generated in human cells. Desirably, the expressed protein is identical to or sufficiently identical to a naturally occurring protein so that immunological responses are reduced or not triggered where the expressed protein is administered on multiple occasions or expressed continually in a patient in need of said protein.

Embodiments of the recombinant vector constructs of the present invention find further utility in the in vitro production of recombinant antibodies and other biologically active proteins for use in therapy or in research. Methods for recombinant protein production are well known in the art and may be utilized for expression of recombinant antibodies using the self processing cleavage site or other protease cleavage site-containing vector constructs described herein.

In one aspect, the invention provides methods for producing a recombinant immunoglobulin or fragment thereof, by introducing an expression vector such as described above into a cell to obtain a transfected cell, wherein the vector comprises in the 5′ to 3′ direction: a promoter operably linked to the coding sequences for immunoglobulin heavy and two light chains or fragment thereof, a self processing sequence between each of said chains. It is appreciated that the coding sequence for either the immunoglobulin heavy chain or the coding sequence for the immunoglobulin light chain may be 5′ (i.e., first) relative to the self processing sequence in a given vector construct.

In an embodiment of a construct for an antibody, the sequence encoding the first or second chain for an antibody or immunoglobulin or a fragment thereof includes a heavy chain or a fragment thereof derived from an IgG, IgM, IgD, IgE or IgA. As broadly stated, the sequence encoding the chain for an antibody or immunoglobulin or a fragment thereof also includes the light chain or a fragment thereof from an IgG, IgM, IgD, IgE or IgA. Embodiments of the invention relate to genes corresponding to proteins for whole antibody molecules as well as modified or derived forms thereof, which include, e.g., other antigen recognition molecules fragments like Fab, single chain Fv (scFv) and F(ab′)₂. The antibodies and fragments can be animal-derived, human-mouse chimeric, humanized, altered by Deimmunisation™ (Biovation Ltd), altered to change affinity for Fc receptors, or fully human. Embodiments of ligand-binding molecules can be affinity maturated as understood in the art. In preferred embodiments, the antibody or other recombinant protein does not elicit or minimally provokes an immune response in a human or animal to which it is administered.

The antibodies can be bispecific and include, but are not limited to, diantibodies, quadroma, mini-antibodies, ScBs antibodies and knobs-into-holes antibodies.

The production and recovery of the antibodies themselves can be achieved in various ways well known in the art (Harlow et al. 1988. Antibodies, A Laboratory Manual, Cold Spring Harbor Laboratory. Other proteins of interest are collected and/or purified and/or used according to methods well known to the art.

In practicing embodiments of the invention, the production of an antibody or variant (analogue) thereof using recombinant DNA technology can be achieved by culturing a modified recombinant host cell under culture conditions appropriate for the growth of the host cell and the expression of the coding sequences. In order to monitor the success of expression, the antibody levels with respect to the antigen may be monitored using standard techniques such as ELISA, RIA and the like. The antibodies are recovered from the culture supernatant using standard techniques known in the art. Purified forms of these antibodies can, of course, be readily prepared by standard purification techniques including but not limited to, affinity chromatography via protein A, protein G or protein L columns, or with respect to the particular antigen, or even with respect to the particular epitope of the antigen for which specificity is desired. Antibodies can also be purified as understood in the art with conventional chromatography, such as an ion exchange, hydrophobic interaction, affinity, or size exclusion column. See also U.S. Pat. No. 5,641,870 by Rinderknecht, et al., Jun. 24, 1997, for “Low pH hydrophobic interaction chromatography for antibody purification” and U.S. Pat. No. 7,427,659 by Shukla, et al., Sep. 23, 2008 for “Process for purifying proteins in a hydrophobic interaction chromatography flow-through fraction,” disclosing purification techniques. The purification techniques can be performed in various combinations or in conjunction with other technologies, such as ammonia sulfate precipitation and size-limited membrane filtration. Where expression systems are designed to include signal peptides, the resulting antibodies are secreted into the culture medium or supernatant; however, intracellular production is also possible. Intracellular contents can be recovered and subject to purification.

Cell culture conditions can be selected that promote a desired level of processing of the polyprotein, e.g., the most complete processing. Such processing could take place intracellularly, but could also take place extracellularly, during or post cell culture process.

The production and selection of antigen-specific, fully human monoclonal antibodies from mice engineered with human Ig loci, has previously been described (Jakobovits et al. 1998. Advanced Drug Delivery Reviews 31:33-42; Mendez et al. 1997. Nature Genetics 15: 146-156; Jakobovits et al. 1995. Curr Opin Biotechnol 6: 561-566; Green et al. 1994. Nature Genetics Vol. 7:13-21).

High level expression of therapeutic monoclonal antibodies has been achieved in the milk of transgenic goats, and it has been shown that antigen binding levels are equivalent to that of monoclonal antibodies produced using conventional cell culture technology. This method is based on development of human therapeutic proteins in the milk of transgenic animals, which carry genetic information allowing them to express human therapeutic proteins in their milk. Once they are produced, these recombinant proteins can be efficiently purified from milk using standard technology. See e.g., Pollock et al. 1999. J. Immunol. Meth. 231:147-157 and Young et al. 1998. Res Immunol. 149(6): 609-610. Animal milk, egg white, blood, urine, seminal plasma and silk worm cocoons from transgenic animals have demonstrated potential as sources for production of recombinant proteins at an industrial scale (Houdebine L M. 2002. Curr Opin Biotechnol 13:625-629; Little et al. 2000. Immunol Today, 21(8):364-70; and Gura T. 2002. Nature, 417:584-5860. The invention contemplates use of transgenic animal expression systems for expression of a recombinant an antibody or variant (analogue) or other protein(s) of interest thereof using the self-processing cleavage site-encoding and/or protease recognition site vectors of the invention.

Production of recombinant proteins in plants has also been successfully demonstrated including, but not limited to, potatoes, tomatoes, tobacco, rice, and other plants transformed by Agrobacterium infection, biolistic transformation, protoplast transformation, and the like. Recombinant human GM-CSF expression in the seeds of transgenic tobacco plants and expression of antibodies including single-chain antibodies in plants has been demonstrated. See, e.g., Streaffield and Howard. 2003. Int. J. Parasitol. 33:479-93; Schillberg et al. 2003. Cell Mol Life Sci. 60:433A5; Pogue et al. 2002. Annu. Rev. Phytopathol. 40:45-74; and McCormick et al. 2003. J Immunological Methods, 278:95-104. The invention contemplates use of transgenic plant expression systems for expression of a recombinant immunoglobulin or fragment thereof or other protein(s) of interest using the protease cleavage site or self-processing cleavage site-encoding vectors of the invention.

Baculovirus vector expression systems in conjunction with insect cells are also gaining ground as a viable platform for recombinant protein production. Baculovirus vector expression systems have been reported to provide advantages relative to mammalian cell culture such as ease of culture and higher expression levels. See, e.g., Ghosh et al. 2002. Mol Ther. 6:5-11, and Ikonomou et al. 2003. Appl Microbiol Biotechnol. 62:1-20. The invention further contemplates use of baculovirus vector expression systems for expression of a recombinant immunoglobulin or fragment thereof using the self-processing cleavage site-encoding vectors of the invention. Baculovirus vectors and suitable host cells are well known to the art and commercially available.

Yeast-based systems may also be employed for expression of a recombinant immunoglobulin or fragment thereof or other protein(s) of interest, including two- or three-hybrid systems, using the self-processing cleavage site-encoding vectors of the invention. See, e.g., U.S. Pat. No. 5,643,745, incorporated by reference herein.

It is understood that the expression cassettes and vectors and recombinant host cells of the present invention which comprise the coding sequences for a self-processing peptide alone or in combination with additional coding sequences for a proteolytic cleavage site find utility in the expression of recombinant immunoglobulins or fragments thereof, proproteins, biologically active proteins and protein components of two- and three-hybrid systems, in any protein expression system, a number of which are known in the art and examples of which are described herein. One of skill in the art may easily adapt embodiments of the vectors, host cells, and methods of the invention for use in any protein expression system.

### EXAMPLE 1

### Lon Protease Inteins and Expression Constructs

Three ATP-dependent Lon protease inteins are reported in the New England Biolabs (NEB, Ipswich, Mass., USA) intein database (InBase, The Intein Database and Registry; at http://www.neb.com/neb/inteins.html). See Perler, F. B. (2002). InBase, the Intein Database. Nucleic Acids Res. 30, 383-384. These inteins were from organisms Pyrococcus abyssi (Pab Lon intein), Pyrococcus furiosus (Pfu Lon intein), and Pyrococcus horikoshii OT3 (Pho Lon intein). These Lon inteins have proposed endonuclease domains, lysines instead of histidines as the penultimate residue of the intein, and different lengths (333, 401, and 474 amino acids, respectively). In the NEB database all three lon inteins are indicated as theoretical inteins, which according to the database indicates that the listing contributor did not indicate that the presence of spliced product had been demonstrated for a given intein entry. It is noted that the endonuclease domain of the Pab Lon intein has been found to not have activity experimentally (Saves I, Morlot C, Thion L, Rolland J L, Dietrich J, Masson J M. Investigating the endonuclease activity of four Pyrococcus abyssi inteins. Nucleic Acids Res. 2002 Oct. 1; 30(19):4158-65).

We have discovered that inteins that are contained in the ATP-dependent protease lon family of genes are very efficient in mediating cleavages of antibody heavy chain and light chains in various single open reading frame construct designs. Sequence information in connection with these inteins is provided herewith.

### Lon Intein Sequences and Vector Construct Designs

Table 1 provides protein sequence information for the Pab Lon intein, Accession No. CAB50486.1 in NCBI/protein, PAB1313 Pab Lon intein, including −1 and +1 extein residues (SEQ ID NO:1).

TABLE 1
Pab Lon intein, amino acid sequence (SEQ ID NO: 1)

QCFSGEETVVIRENGEVKVLRLKDFVEKALEKPSGEGLDGDVKVVYHDF
RNENVEVLTKDGFTKLLYANKRIGKQKLRRVVNLEKDYWFALTPDHKVY
TTDGLKEAGEITEKDELISVPITVFDCEDEDLKKIGLLPLTSDDERLRK
IATLMGILFNGGSIDEGLGVLTLKSERSVIEKFVITLKELFGKFEYEII
KEENTILKTRDPRIIKFLVGLGAPIEGKDLKMPWWVKLKPSLFLAFLEG
FRAHIVEQLVDDPNKNLPFFQELSWYLGLFGIKADIKVEEVGDKHKIIF
DAGRLDVDKQFIETWEDVEVTYNLTTEKGNLLANGLFVKNS

Table 2 describes a nucleotide sequence for the Pab Lon intein which has been optimized for codon usage.

TABLE 2
Pab Lon intein, nucleotide sequence (SEQ ID NO: 2)

tgcttcagcggcgaggaaaccgtggtgatccgggagaacggcgaggtga
aggtgctgcggctgaaggacttcgtggagaaggccctggaaaagccctc
cggcgagggcctggacggcgacgtgaaagtggtgtaccacgacttccgg
aacgagaacgtggaggtgctgaccaaggacggcttcaccaagctgctgt
acgccaacaagcggatcggcaagcagaaactgcggcgggtggtgaacct
ggaaaaggactactggttcgccctgacccccgaccacaaggtgtacacc
accgacggcctgaaagaggccggcgagatcaccgagaaggacgagctga
tcagcgtgcccatcaccgtgttcgactgcgaggacgaggacctgaagaa
gatcggcctgctgcccctgaccagcgacgacgagcggctgcggaagatc
gccaccctgatgggcatcctgttcaacggcggcagcatcgatgagggcc
tgggcgtgctgaccctgaagagcgagcggagcgtgatcgagaagttcgt
gatcaccctgaaagagctgttcggcaagttcgagtacgagatcatcaaa
gaggaaaacaccatcctgaaaacccgggacccccggatcatcaagtttc
tggtgggcctgggagcccccatcgagggcaaggatctgaagatgccttg
gtgggtgaagctgaagcccagcctgttcctggccttcctggaaggcttc
cgggcccacatcgtggagcagctggtcgacgaccccaacaagaatctgc
ccttctttcaggaactgagctggtatctgggcctgttcggcatcaaggc
cgacatcaaggtggaggaagtgggcgacaagcacaagatcatcttcgac
gccggcaggctggacgtggacaagcagttcatcgagacctgggaggatg
tggaggtgacctacaacctgaccacagagaagggcaatctgctggccaa
cggcctgttcgtgaagaac

Table 3 describes the protein sequence, SEQ ID NO:3, encoded by SEQ ID NO:2.

TABLE 3
Pab Lon intein, amino acid sequence (SEQ ID NO: 3).

CFSGEETVVIRENGEVKVLRLKDFVEKALEKPSGEGLDGDVKVVYHDFR
NENVEVLTKDGFTKLLYANKRIGKQKLRRVVNLEKDYWFALTPDHKVYT
TDGLKEAGEITEKDELISVPITVFDCEDEDLKKIGLLPLTSDDERLRKI
ATLMGILFNGGSIDEGLGVLTLKSERSVIEKFVITLKELFGKFEYEIIK
EENTILKTRDPRIIKFLVGLGAPIEGKDLKMPWWVKLKPSLFLAFLEGF
RAHIVEQLVDDPNKNLPFFQELSWYLGLFGIKADIKVEEVGDKHKIIFD
AGRLDVDKQFIETWEDVEVTYNLTTEKGNLLANGLFVKN

Table 4 provides protein sequence information for the Pfu Lon intein, Accession No. AAL80591.1 in NCBI/protein, PF0467, including −1 and +1 extein residues (SEQ ID NO:4).

TABLE 4
Pfu Lon intein, amino acid sequence (SEQ ID NO: 4).

QCFSGEEVILIEKDGEKKVFKLREFVDGLLKEASGEGMDGSIRVVYKDL
QGENIKILTKDGLVKLLYVNRREGKQKLRKIVNLEKDYWLALTPEHKVY
TIKGLKEAGEITKDDEIIRVPLTILDGFDVAEKSIREELERLSLLPLNS
EDSRLEKIAGIMGALFGSGGIDENLNTLSFVSSEKKTIEQFVKALSELF
GEFDYKIEEKENSIIFRTCDKRIVTFFATLGAPVGDKSKVKLKLPWWVK
LKPSLFLAFMDGLYSSNRNDKEILEITQLTDNVETFFEEISWYLSFFGI
KAEAEEDEEKDKYRARLTLSSSIDNMLNFIEFIPISFSPAKREKFFKEI
EKYLEYSIPEKTEDLKKRVKRVKKGERRNFLESWEEVEVTYNVTTETGN
LLANGLFVKNS

Table 5 provides nucleotide sequence information for the native Pfu Lon intein (SEQ ID NO:5).

TABLE 5
Pfu Lon intein, nucleotide sequence (SEQ ID NO: 5)

tgttttagcggtgaagaagttatcttaattgaaaaggacggagagaaaa
aagtcttcaaacttagggagttcgttgacggtctccttaaggaggcgtc
tggagaagggatggacggaagtattagagtagtttataaagatcttcaa
ggggaaaacataaaaatactcacaaaagacggacttgtaaagctccttt
atgtcaatagaagagaagggaagcaaaagcttagaaaaatagtaaatct
tgaaaaggattattggcttgcattaacacctgaacataaagtgtacaca
ataaagggccttaaagaagctggagagataactaaagatgatgagataa
taagagtgcctctcacaattcttgacggctttgacgtagccgagaagag
tataagagaggaacttgaaaggcttagcctacttccactaaatagtgaa
gacagtagactagaaaagatagcaggaatcatgggcgcactctttggta
gtggaggtatcgatgagaatctcaatacccttagctttgtttctagcga
gaagaaaacaattgaacagtttgttaaagcactcagcgagctcttcggg
gaatttgactataaaattgaagaaaaagaaaacagcattattttcagaa
catgtgataaaagaatagtgaccttctttgctacacttggtgcaccagt
tggagacaaaagcaaagttaagcttaagcttccatggtgggtcaagctt
aagccgtcacttttcctcgccttcatggatggtctctacagtagcaata
ggaatgacaaagaaatcctcgaaataactcaacttactgacaacgtcga
aacgttcttcgaggaaatatcttggtatctgagcttctttggaattaag
gcagaagctgaagaggatgaagaaaaagataaatacagggctagactta
cgctatcctcatcaatagacaacatgcttaatttcattgagttcattcc
aataagcttttctccagcaaagagagaaaaattctttaaggaaattgaa
aaatatctggaatatagcattcccgaaaagactgaggatcttaagaaac
gagttaagagagttaagaagggagagagaaggaatttcctcgaaagctg
ggaggaagttgaagttacttacaacgtaactacagagacaggaaatcta
cttgctaacggtctatttgttaagaac

Table 6 describes the protein sequence, SEQ ID NO:6, encoded by SEQ ID NO:5.

TABLE 6
Pfu Lon intein, amino acid sequence

CFSGEEVILIEKDGEKKVFKLREFVDGLLKEASGEGMDGSIRVVYKDLQG
ENIKILTKDGLVKLLYVNRREGKQKLRKIVNLEKDYWLALTPEHKVYTIK
GLKEAGEITKDDEIIRVPLTILDGFDVAEKSIREELERLSLLPLNSEDSR
LEKIAGIMGALFGSGGIDENLNTLSFVSSEKKTIEQFVKALSELFGEFDY
KIEEKENSIIFRTCDKRIVTFFATLGAPVGDKSKVKLKLPWWVKLKPSLF
LAFMDGLYSSNRNDKEILEITQLTDNVETFFEEISWYLSFFGIKAEAEED
EEKDKYRARLTLSSSIDNMLNFIEFIPISFSPAKREKFFKEIEKYLEYSI
PEKTEDLKKRVKRVKKGERRNFLESWEEVEVTYNVTTETGNLLANGLFVK
N

The Pfu Lon intein was cloned using PCR techniques. The Pab Lon intein nucleotide sequence was synthesized by a design according to mammalian codon usage. The protein sequence of the Pyrococcus abysii lon protease intein was obtained from Inbase, a publicly curated database of inteins sponsored by New England Biolabs, Ipswich, Mass. (http://www.neb.com/neb/inteins.html). The protein sequence obtained is listed as EMBL accession number CAB50486.1, gi5459000; however, the protein sequence as listed on the web site was used. The Pab-lon intein protein sequence is indicated in Table 7.

TABLE 7
Pab-Ion intein amino acid sequence, (SEQ ID NO: 7)

CFSGEETVVIRENGEVKVLRLKDFVEKALEKPSGEGLDGDVKVVYHDFRN
ENVEVLTKDGFTKLLYANKRIGKQKLRRVVNLEKDYWFALTPDHKVYTTD
GLKEAGEITEKDELISVPITVFDCEDEDLKKIGLLPLTSDDERLRKIATL
MGILFNGGSIDEGLGVLTLKSERSVIEKFVITLKELFGKFEYEIIKEENT
ILKTRDPRIIKFLVGLGAPIEGKDLKMPWWVKLKPSLFLAFLEGFRAHIV
EQLVDDPNKNLPFFQELSWYLGLFGIKADIKVEEVGDKHKIIFDAGRLDV
DKQFIETWEDVEVTYNLTTEKGNLLANGLFVKN

This Pab-lon protein sequence was back-translated to a DNA sequence optimized for mammalian expression by GeneArt (GeneArt AG, Regensburg, Germany) using a proprietary method. The resulting Pab-lon intein DNA sequence is indicated in Table 8. It would be appreciated that DNA constructs may optionally provide additional flanking adapters depending on the selection of particular cloning and expression vectors and corresponding molecular biology approaches using conventional techniques. The DNA sequence was synthesized (GeneArt Synthesis Number 0611467) as a 999 bp fragment and delivered in the GeneArt vector plasmid, pGA4. See the Registry of Biological Standard Parts at http://partsregistry.org, including Part:BBa_J70003. The DNA material received was resequenced and determined to correspond to the designed sequence. This DNA material was used directly as a source/template for subsequent Pab-lon intein containing plasmid constructs; the plasmid was not repropagated.

TABLE 8
Pab-Ion intein nucleic acid sequence (SEQ ID NO:
8).

TGCTTCAGCGGCGAGGAAACCGTGGTGATCCGGGAGAACGGCGAGGTGAA
GGTGCTGCGGCTGAAGGACTTCGTGGAGAAGGCCCTGGAAAAGCCCTCCG
GCGAGGGCCTGGACGGCGACGTGAAAGTGGTGTACCACGACTTCCGGAAC
GAGAACGTGGAGGTGCTGACCAAGGACGGCTTCACCAAGCTGCTGTACGC
CAACAAGCGGATCGGCAAGCAGAAACTGCGGCGGGTGGTGAACCTGGAAA
AGGACTACTGGTTCGCCCTGACCCCCGACCACAAGGTGTACACCACCGAC
GGCCTGAAAGAGGCCGGCGAGATCACCGAGAAGGACGAGCTGATCAGCGT
GCCCATCACCGTGTTCGACTGCGAGGACGAGGACCTGAAGAAGATCGGCC
TGCTGCCCCTGACCAGCGACGACGAGCGGCTGCGGAAGATCGCCACCCTG
ATGGGCATCCTGTTCAACGGCGGCAGCATCGATGAGGGCCTGGGCGTGCT
GACCCTGAAGAGCGAGCGGAGCGTGATCGAGAAGTTCGTGATCACCCTGA
AAGAGCTGTTCGGCAAGTTCGAGTACGAGATCATCAAAGAGGAAAACACC
ATCCTGAAAACCCGGGACCCCCGGATCATCAAGTTTCTGGTGGGCCTGGG
AGCCCCCATCGAGGGCAAGGATCTGAAGATGCCTTGGTGGGTGAAGCTGA
AGCCCAGCCTGTTCCTGGCCTTCCTGGAAGGCTTCCGGGCCCACATCGTG
GAGCAGCTGGTCGACGACCCCAACAAGAATCTGCCCTTCTTTCAGGAACT
GAGCTGGTATCTGGGCCTGTTCGGCATCAAGGCCGACATCAAGGTGGAGG
AAGTGGGCGACAAGCACAAGATCATCTTCGACGCCGGCAGGCTGGACGTG
GACAAGCAGTTCATCGAGACCTGGGAGGATGTGGAGGTGACCTACAACCT
GACCACAGAGAAGGGCAATCTGCTGGCCAACGGCCTGTTCGTGAAGAAC

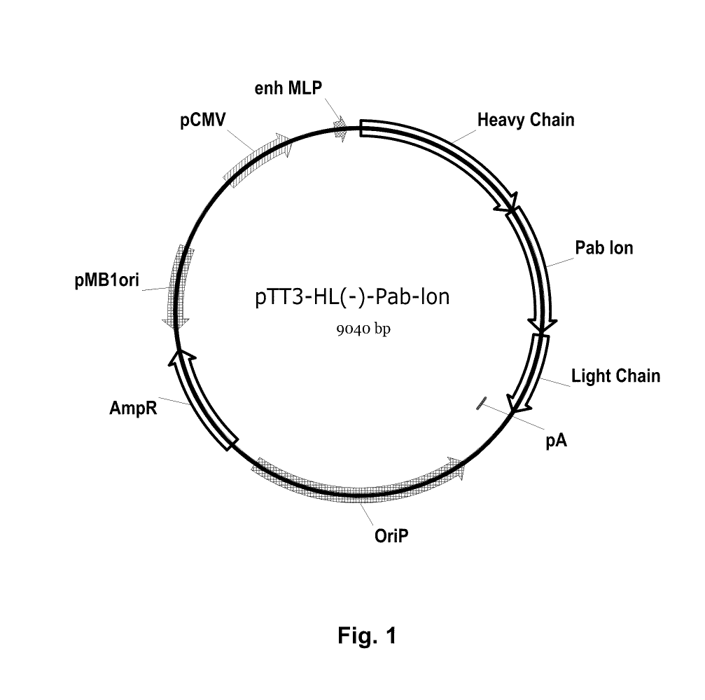

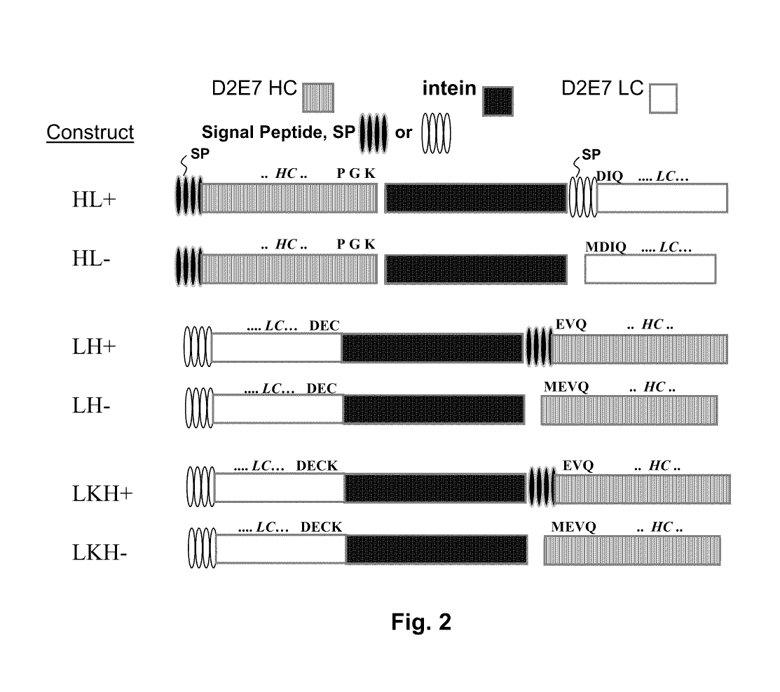

The following mammalian expression vectors were constructed: pTT3-pfu lon HL(+), pTT3-pfu lon HL(−), pTT3-pfu lon LH(+), pTT3-pfu lon LH(−), pTT3-pfu lon LKH(+), pTT3-pfu lon LKH(−), pTT3-pab lon HL(+), pTT3 pab lon HL (−), pTT3-pab lon LH (+), pTT3-pab lon LH(−), pTT3-pab lon LKH(+), pTT3-pab lon LKH(−). Here, the H and L components represent the immunoglobulin heavy and light chains for the antibody designated D2E7. For a schematic representation of the pTT3 pab lon HL (−) construct, see FIG. 1. FIG. 2 illustrates aspects of the structures for the sORF components of these transient expression vectors that are capable of expressing the D2E7 antibody.

Although the pTT3 vector represents a particular embodiment, further embodiments can include aspects pertaining to an isolated nucleic acid encoding one or more proteins disclosed herein. A further embodiment provides a vector comprising an isolated nucleic acid sequence wherein said vector is selected from the group consisting of pcDNA; pTT (Durocher et al., Nucleic Acids Research 2002, Vol 30, No. 2:E9); pTT3; pEFBOS (Mizushima, S. and Nagata, S., 1990, Nucleic Acids Research Vol 18, No. 17:5322); pBV; pJV; and pBJ. As noted above, various constructs were made on the pTT3 vector backbone. This vector has EBV origin of replication, which allows for its episomal amplification in tranfected 293E cells (which express Epstein-Barr virus nuclear antigen 1) in suspension culture. See Durocher et al., describing the vector pTT. Relative to pTT, pTT3 has an additional multiple cloning site as indicated in US Patent Application Publication 20050147610 by Ghayur, Tariq et al., Jul. 7, 2005.

Each pTT3-based vector had one ORF which was regulated by a CMV promoter. In the ORF, the intein sequence was inserted in frame between the antibody heavy and light chains (HC and LC, or simply H and L, respectively), either in the order of HC-intein-LC or LC-intein-HC. The constructs with “HL” designation have the antibody HC coding sequence followed by intein and then by LC coding sequence; constructs with “LH” designation have the LC coding sequence followed by intein and then by HC coding sequence. Constructs with “LKH” designation have a lysine (K) inserted between the LC and intein. The constructs with the “(−)” designation have one signal peptide at the beginning of the ORF and a methionine inserted between the last amino acid of the intein and the first amino acid of the mature antibody heavy or light chain that follow the intein. Constructs with the “(+)” designation have one signal peptide at the beginning of the ORF and a second signal peptide at the beginning of the antibody subunit that is downstream of the intein.

The constructs were introduced into 293E cells through transient transfection. Briefly, complexes were prepared using pTT3 vectors encoding the ORF constructs and polyethylenimine (PEI). PEI-DNA complexes were used to transfect HEK293E cells; see Durocher et al., 2002, Nucl. Acids Res. 30:E9. Cells and culture supernatants were collected four to seven days after transfection for analysis.

Protein expression by constructs. In multiple transient expression experiments, the culture supernatant samples were collected on the seventh or eighth day post-transfection. The samples assessed by ELISA and contained the levels or ranges of secreted antibody from measurements of IgG as shown below.

TABLE 9
Lon intein immunoglobulin sORF
constructs antibody production.

| | |
|---|---|
| | IgG (secreted), |
| Construct | μg/ml |

sORF constructs

| | |
|---|---|
| pTT3-pfu lon HL (+) | 1.4-2.1 |
| pTT3-pfu lon HL (−) | 31-40 |
| pTT3-pfu lon LH (+) | <0.1 |
| pTT3-pfu lon LH (−) | 1.6 |
| pTT3-pfu lon LKH (+) | <0.1 |
| pTT3-pfu lon LKH (−) | 10 |
| pTT3-pab lon HL (+) | 1.3 |
| pTT3-pab lon HL (−) | 41-68 |
| pTT3-pab lon LH (+) | <0.1 |
| pTT3-pab lon LH (−) | 0.5 |
| pTT3-pab lon LKH (+) | <0.1 |
| pTT3-pab lon LKH (−) | 0.9 |

Other construct

| | |
|---|---|
| Control vector | 10-60 |

A conventional two-vector system expressing the same antibody as the construct series described above, and using the same regulatory elements, was included in these experiments as a control (see Table, bottom row). Thus the control vector expressed the D2E7 antibody using a conventional approach of introducing the antibody heavy and the light chains from two separate ORFs carried in two separate pTT3 vectors. The antibody secretion level produced from this control vector system ranged from 10 to 60 μg/ml as indicated in the table.

The IgG secretion level produced by several of the sORF construct designs using the Lon inteins are in the same range, or higher, compared to that produced using the conventional control vector. These levels are significantly higher than those produced using the “2A” technology, which was reported to be at 1.6 μg/ml in mammalian cells (Fang et al., 2005, Nature Biotechnology 23:584-590). While both Pab Lon and Pfu Lon inteins could be used in construct designs to yield desirable levels of antibody production, the Pab Lon intein in the described pTT3 constructs allowed for a higher level of a higher level of antibody secretion. These data also suggest that antibody secretion levels are generally greater when an HL construct design is used than when a LH construct design is used. By combining the feature of the order of immunoglobulin chains and aspects of signal sequences, HL(−) constructs were able to generate the highest levels of secreted antibody product among those studied.

### Further Characterization of Expression Products

Certain sORF constructs listed in Table 9 were further characterized regarding aspects of expression including analysis of expression products. These constructs included four examples which produced relatively higher levels of secreted antibody: pTT3 pfu lon HL (−), pTT3 pfu lon HL (+), pTT3 pfu lon LKH (−), and pTT3 pab lon HL (−). The secreted antibody produced from these constructs was purified by protein A affinity chromatography and analyzed on both reducing and non-reducing SDS-PAGE gels, and the N-terminal amino acid sequences for their HL and LC were determined.

Samples produced using pTT3 pfu lon HL (−) contained gel migration bands corresponding to the antibody HC, antibody LC, and fully assembled antibody (on non-reducing gels), with migrations indistinguishable from antibody produced by traditional methods with conventional vector such as the control D2E7 vector described above. On reducing gels, in addition to the bands corresponding to the antibody HC and LC, there were also two higher molecular weight (MW) bands that appeared to correspond to the unprocessed tripartite protein (HC-intein-LC) and partially processed HC-intein fusion. This assessment was based on western blot analysis and mass spectrometry analysis. The abundance of these two bands appeared to be dependent on culture conditions and can be reduced by modifying culture conditions. These higher MW products can be conveniently removed from the fully processed antibody drug substance using methods according to other description provided herein and/or as would be understood in the art.

Samples produced using pTT3 pfu lon HL (+) contained bands corresponding to antibody HC, antibody LC, and full antibody (on non-reducing gels), with migrations indistinguishable from antibody produced by traditional methods. In addition there was one larger MW band corresponding to the tripartite polyprotein. Samples produced using pTT3 pfu lon LKH (−) also contained bands corresponding to HC, LC, and full antibody (on non-reducing gels) with migrations indistinguishable from antibody produced using conventional vectors. On reducing gels, in addition to the bands corresponding to the HC and LC, there were also two higher MW bands. The first one of these corresponded to the tripartite polyprotein, as described above for other vector designs; the second band corresponded to LC-intein fusion product, resulting from incomplete cleavage at this junction. In terms of relative abundance of products, there appeared to be as much LC-intein fusion as cleaved LC.

Samples produced using pTT3 pab lon HL (−) contained bands corresponding to HC, LC, and full antibody (on non-reducing gels) with migrations indistinguishable from antibody produced by traditional methods. On reducing gels, in addition to the bands corresponding to the HC and LC, there was one major higher MW band that appeared to correspond to the unprocessed tripartite protein based on western blot analysis. Compared to samples produced using pTT3 pfu lon HL(−), there was a relatively smaller amount of this tripartite higher MW band. This result suggests that even though Pfu lon intein and Pab lon intein are homologous and functionally similar in our vector designs, Pab lon mediated N-terminal cleavage is more complete than that mediated by Pfu lon, as there is little HC-intein fusion observed following expression from the Pab lon construct. It is noted, however, that both constructs can yield fully assembled antibody product.

On one hand, certain protein outputs like the unprocessed and partially processed proteins may be considered contaminant products relative to other construct output such as the fully processed and fully self-assembled antibody product. On the other hand, such certain protein outputs may be useful, for example as material for further processing reactions and/or directed assembly which can yet generate full antibody product. If these protein outputs are viewed as contaminant byproducts, then as noted there are options and approaches to facilitate removal and thus enrich or purify for a desired component such as the full antibody.

In addition to extracellular samples of culture supernatant from various construct expression systems, intracellular samples were also obtained and analyzed by western blot analysis using detection antibodies with specificities against both HC and LC. Similar protein species were observed as described for those species in the cultured supernatant samples.

The N-terminal amino acid sequences of both heavy chain and light chain products of various constructs were determined (see Table below). The results signified that intein-mediated protein cleavages took place at precisely the two splicing junctions, namely at the junction of the HC and intein components and at the junction of the LC and intein components.

TABLE 10
Heavy and light chain N-terminal amino acid sequences
of major species of expression products from sORF constructs.

| | HC, | | LC, | |
|---|---|---|---|---|
| | N-term, AA | SEQ | N-term, AA | SEQ |
| Construct | sequence | ID NO: | sequence | ID NO: |
| control, mature HC or LC | EVQLVESGGG | 9 | DIQMTQSPSS | 11 |
| sORF constructs | | | | |
| pTT3 pfu Ion HL (+) | EVQLVESGGG | 9 | DIQMTQSPSS | 11 |
| pTT3 pfu Ion HL (−) | EVQLVESGGG | 9 | MDIQMTQSPS | 12 |
| pTT3 pfu Ion LKH (−) | MEVQLVESGG | 10 | DIQMTQSPS | 11 |
| pTT3 pab Ion HL (−) | EVQLVESGGG | 9 | MDIQMTQSPS | 12 |

Functional Properties of IgG1 Antibody from Lon Intein Construct

The secreted D2E7 antibody products from sORF construct designs of Table 9 were also analyzed by antigen-specific ELISA. The results of the analysis demonstrated that the construct antibody products bind human TNFalpha, the ligand of the D2E7 antibody. Thus the intein-based constructs and expression systems are capable of expressing and generating sORF products which yield fully self-assembled multimeric antibody which is functional and antigen-specific.

Antibody produced using pTT3 pfu lon HL(−) construct was purified by protein A affinity purification followed by SEC, size exclusion chromatography. The purified antibody was analyzed by surface plasmon resonance technology using a BiaCore™ system. Characterization of the sORF construct output included aspects of its binding to the relevant ligand, TNFα. In Table 11 the results of values are indicated from the BiaCore analysis regarding kinetic parameters of the association rate constant (ka, units of 1/Ms); dissociation rate constant (kd, units of 1/s), and the equilibrium dissociation constant (KD, units of M). The dissociation constant value (KD) is understood to be similar to that for adalimumab (D2E7) antibody produced using a conventional vector (with two distinct immunoglobulin chain ORFs).

TABLE 11
Kinetic parameters of antibody produced from sORF construct.

| Construct | ka (1/Ms) | kd (1/s) | KD (M) |
|---|---|---|---|
| pTT3 pfu lon HL(−) | 1.51E+06 | 1.10E−04 | 7.29E−11 |

### EXAMPLE 2

### sORF Constructs Producing Antibody with Variations of Light Chain Sequences

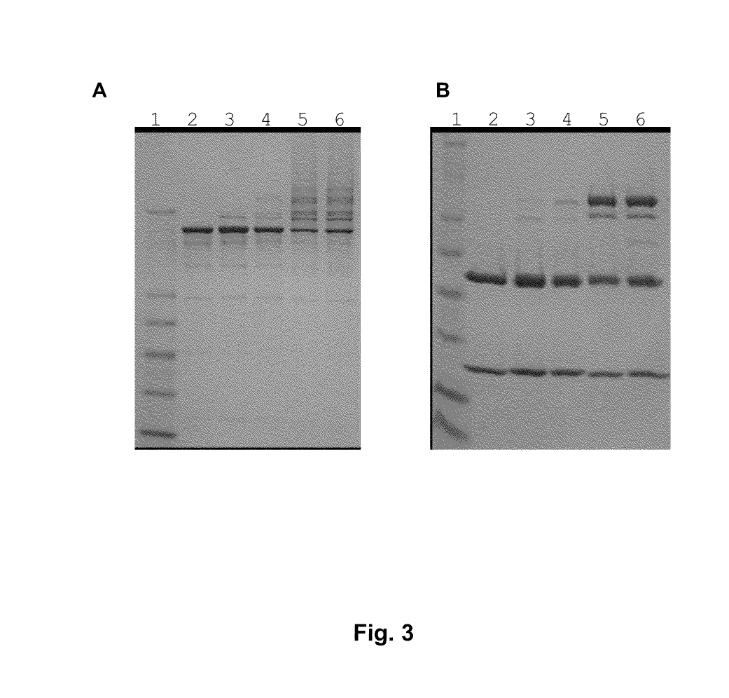

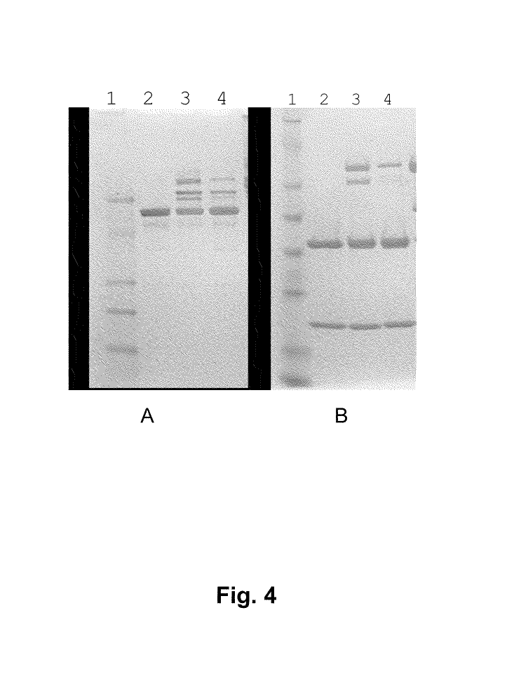

Several sORF constructs were generated with light chain sequences that are variations from the immunoglobulin light chain of the D2E7. These sORF constructs were engineered with the heavy chain also as in D2E7 and thus were capable of producing IgG1 antibody material. Using constructs pTT3 pfu lon HL (−) and pTT3 pab lon HL (−) as backbones, we generated and tested constructs with sequence variations at the C-terminal splicing junction, i.e., the junction between the intein and the downstream immunoglobulin light chain component (see Table 12). The secreted immunoglobulins of certain constructs were purified by Protein A affinity purification and analyzed on reducing and non-reducing SDS-PAGE gels. Intracellular samples were also analyzed by western blot analysis using antibodies against both HC and LC. See, for example, FIG. 3 and FIG. 4.

FIG. 3 illustrates results of an SDS-PAGE gel for protein analysis of sORF expression products. Secreted IgG molecules were purified by Protein A affinity chromatography and separated on SDS-PAGE gels under non-reducing (A) and reducing conditions (B). Lanes and samples from left to right are: (Lane 1) MW markers; (2) control construct product, D2E7 antibody from non-sORF expression system; (3) Pab-lon mut A1; (4) Pab-lon mut A2; (5) pTT3 pfu lon YP, and (6) pTT3 pfu lon MA.

FIG. 4 illustrates results of an SDS-PAGE gel for protein analysis of further sORF expression products. Secreted IgGs were purified by Protein A affinity chromatography and separated on SDS-PAGE gels under non-reducing (A) and reducing (B) conditions. Lanes and samples from left to right are: (Lane 1) MW markers; (2) control D2E7 product; (3) pTT3 pfu lon HL (−); and (4) pTT3 pfu lon MutA.

TABLE 12
AA Sequences in sORF constructs at C-terminal
splicing junctions of intein-LC.

| | | SEQ ID | C-term. | SEQ ID | | SEQ ID |
|---|---|---|---|---|---|---|
| Construct | Intein | NO: | junction | NO: | Mature LC | NO: |
| pTT3 pfu Ion HL (−) | ANGLFVKN | 13 | M | | DIQMTQS | 17 |
| pTT3 pfu Ion MutA | ANGLFVKN | 13 | MRAKR | 14 | DIQMTQS | 17 |
| pTT3 pfu Ion MutB | ANGLFVKN | 13 | — | | DIQMTQS | 17 |
| pTT3 pfu Ion YP | ANGLFVKN | 13 | YP | | DIQMTQS | 17 |
| pTT3 pfu Ion RP | ANGLFVKN | 13 | RP | | DIQMTQS | 17 |
| pTT3 pfu Ion VP | ANGLFVKN | 13 | VP | | DIQMTQS | 17 |
| pTT3 pfu Ion QP | ANGLFVKN | 13 | QP | | DIQMTQS | 17 |
| pTT3 pfu Ion AP | ANGLFVKN | 13 | AP, | | DIQMTQS | 17 |
| pTT3 pfu Ion HA | ANGLFVKN | 13 | HA | | DIQMTQS | 17 |
| pTT3 pfu Ion YA | ANGLFVKN | 13 | YA | | DIQMTQS | 17 |
| pTT3 pfu Ion MP | ANGLFVKN | 13 | MP | | DIQMTQS | 17 |
| pTT3 pfu Ion MA | ANGLFVKN | 13 | MA | | DIQMTQS | 17 |
| pTT3 pab Ion MutA1 | ANGLFVKN | 13 | HA RGVFRR | 15 | DIQMTQS | 17 |
| pTT3 pab Ion MutA2 | ANGLFVKN | 13 | MD RGVFRR | 16 | DIQMTQS | 17 |
| pTT3 pab Ion AIQ | ANGLFVKN | 13 | — | | AIQMTQS | 18 |
| pTT3 pab Ion NIQ | ANGLFVKN | 13 | — | | NIQMTQS | 19 |
| pTT3 pab Ion NFQ | ANGLFVKN | 13 | — | | NFQMTQS | 20 |

The immunoglobulin secretion levels produced by the constructs of Table 12 are indicated in Table 13. The amino acids at the N-termini of the mature light chains were determined and the results of characterizing partial sequences are shown.

TABLE 13
IgG levels and N-terminal protein sequence of light chains
in antibodies from sORF constructs.

| | IgG | N-terminal amino acid | SEQ ID |
|---|---|---|---|
| Construct | (ug/ml) | sequence of LC | NO: |
| pTT3 pfu Ion HL (−) | | M DIQMTQS | 21 |
| pTT3 pfu Ion MutA | 17 | MRAKR DIQMTQS | 22 |
| pTT3 pfu Ion MutB | 6 | DIQMTQS | 17 |
| pTT3 pfu Ion YP | 32 | YP DIQMTQS | 23 |
| pTT3 pfu Ion RP | 22 | RP DIQMTQS | 24 |
| pTT3 pfu Ion VP | 20 | VP DIQMTQS | 25 |
| pTT3 pfu Ion QP | 13 | QP DIQMTQS | 26 |
| pTT3 pfu Ion AP | 21 | AP DIQMTQS | 27 |
| pTT3 pfu Ion HA | 18 | HA DIQMTQS | 28 |
| pTT3 pfu Ion YA | 15 | YA DIQMTQS | 29 |
| pTT3 pfu Ion MP | 29 | MP DIQMTQS | 30 |
| pTT3 pfu Ion MA | 33 | MA DIQMTQS | 31 |
| pTT3 pab Ion MutA1 | 16 | HA RGVFRR DIQMTQS | 32 |
| pTT3 pab Ion MutA2 | 11 | MD RGVFRR DIQMTQS | 33 |
| pTT3 pab Ion AIQ | 24 | AIQMTQS | 18 |
| pTT3 pab Ion NIQ | 20 | NIQMTQS | 19 |
| pTT3 pab Ion NFQ | 18 | NFQMTQS | 20 |

### Results

For these constructs, the use of different AA residues at the +1 position (immediately following intein) appeared to be a factor in the yield of antibody secreted. The use of amino acid residues H, Y, R, V, Q, A, N, and M at this position yielded relatively higher levels of antibody expression. The analysis of the light chains for their N-terminal amino acids (Table 13) suggested complete and precise cleavage at the C-terminal end of the intein. Similar to antibodies produced by constructs pTT3 pfu lon HL(−) and pTT3 pab lon HL(−), processed HC and LC, as well assembled full antibody represented the majority of secreted protein species. When the amino acid aspartate (Asp; D) was used directly following the intein as in construct pTT3 pfu lon MutB, however, there was little antibody secretion. The intracellular proteins produced by this construct were analyzed. It was determined that when D is the first amino acid following the intein, there is little cleavage at the C-terminal splicing junction, yielding little antibody LC, and a relatively large amount of intein-LC fusion protein.

Amino acid sequences of the mature germ line light chain of the kappa isotype variable region (Vₖₐₚₚₐ) generally start with, D, E, N, A, or V; that of lambda isotype (V_{lambda}) generally start with Q, S, L, or N. From our results, embodiments of sORF vectors using Pab lon or Pfu lon inteins for production of antibodies include those having LC starting with any amino acids. In preferred embodiments, for purposes of achieving higher efficiency of overall complete processing, the LC start with an amino acid other than D or E although such amino acids can serve as operative options.

We found that the region between the intein and the mature antibody LC downstream from the intein appears to contribute to the efficiency of the cleavage at the N-terminal splicing junction. For example, we compared the output of constructs pTT3 pfu lon HL (−) and pTT3 pfu lon MutA. While the cleavage at the N-terminal splicing junction is not complete when pTT3 pfu lon HL (−) is used, yielding some partially processed HC-intein fusion protein, the amount of this protein species is significantly decreased when construct pTT3 pfu lon MutA is used instead (see FIG. 2). Thus while various constructs may be useful in generating desirable products, certain constructs may have attributes such as the ability to generate relatively higher yields of particularly desired products, e.g., fully processed and self-assembled multimeric secreted antibodies.

### EXAMPLE 3

### Further Options for Intein Components of sORF Constructs

Inteins of klbA Genes from Methanococcus Jannaschii and Pyrococcus Abyssi

We explored further intein options for sORF constructs including inteins of klbA genes such as from Methanococcus and Pyrococcus species. We discovered that inteins that are contained in the klbA gene are also efficient options for mediating protein expression and processing, including in the context of cleavages of antibody heavy chain and light chains in various single open reading frame construct designs.

In particular we examined klbA inteins from Methanococcus jannaschii (Mja klbA intein), Pyrococcus abyssi (Pab klbA intein), and Pyrococcus furiosus (Pfu klbA intein). Inteins from the first two organisms are mini-inteins, lacking endonuclease domains, whereas Pfu klbA is a full size intein. The sequence lengths of the native intein protein segments are 168, 333, and 522 amino acids, respectively. Sequence information in connection with these inteins is provided in tables below. Inteins, modified inteins, and constructs are thus developed for expression systems.

### KlbA Intein Sequences and Vector Construct Designs

The nucleotide sequence of Mja klbA was modified to allow for relative optimization of mammalian codon usage. Table 14 provides nucleic acid sequence information for the Mja klbA intein gene of Methanococcus jannaschii which has been so modified (SEQ ID NO:34). Table 15 provides protein sequence information for the Mja KlbA intein segment. See also Accession No. Q58191 in NCBI/protein, MJ0781. Table 16 provides nucleic acid sequence information for the Pab klbA intein gene which was modified in the aspect of codon usage relative to the native sequence. For the native sequence, see Accession No. [B75050 in NCBI/protein, PAB1457] of the NEB Inbase information for Pab KlbA Intein. According to this source, the indicated protein amino acid sequence is indicated as including −1 and +1 extein residues which appear to be G and C, respectively. Table 17 provides the amino acid sequence information for the Pab KlbA intein protein segment. Table 18 provides nucleic acid sequence information for the Pfu klbA intein gene (native). Table 19 provides the amino acid sequence information for the Pfu KlbA intein protein segment; see also Accession No. AE010211 in NCBI.

TABLE 14
Mja klbA intein gene, nucleotide sequence
(SEQ ID NO: 34), codon usage modified.

Gctctggcctacgacgagcccatctacctgagcgacggcaacatcatcaa
catcggcgagttcgtggacaagttcttcaagaagtacaagaacagcatca
agaaagaggacaacggcttcggctggatcgacatcggcaacgagaacatc
tacatcaagagcttcaacaagctgtccctgatcatcgaggacaagcggat
cctgagagtgtggcggaagaagtacagcggcaagctgatcaagatcacca
ccaagaaccggcgggagatcaccctgacccacgaccaccccgtgtacatc
agcaagaccggcgaggtgctggaaatcaacgccgagatggtgaaagtggg
cgactacatctatatccccaagaacaacaccatcaacctggacgaggtga
tcaaggtggagaccgtggactacaacggccacatctacgacctgaccgtg
gaggacaaccacacctacatcgccggcaagaacgagggcttcgccgtgag
caac

TABLE 15
Mja KlbA intein protein, amino acid sequence
(SEQ ID NO: 35)

ALAYDEPIYLSDGNIINIGEFVDKFFKKYKNSIKKEDNGFGWIDIGNENI
YIKSFNKLSLIIEDKRILRVWRKKYSGKLIKITTKNRREITLTHDHPVYI
SKTGEVLEINAEMVKVGDYIYIPKNNTINLDEVIKVETVDYNGHIYDLTV
EDNHTYIAGKNEGFAVSN

TABLE 16
Pab klba intein gene, nucleotide sequence (SEQ ID
NO: 36), codon usage modified.

Gctctgtactacttcagcgagatccagctgcccaacggcaaagagttcat
cggcaaactggtggacgagctgttcgagaagtaccacgacaagatcggca
agtacaaggacatggaatacgtggagctgaacgaagaggacaccttcgag
gtgatcagcatcggccccgacctgagcgccaggcggcacaaggtgaccca
cgtgtggcggcggaaggtgaaagacggcgagaagctggtgaagatccgga
ccgccagcggcaaagaactggtgctgacccaggaccaccccgtgttcgtg
ctgctgggccgggacgtggccagacgggacgccggcaacgtgaaagtggg
cgacgagatcgccgtgctgaacaccaggcccgacttcagcgtgctgtccc
cccctgccatgcccgagctgctgtccgagcccttcaactacgagctgtcc
agcatcggcgacgtggcctgggacgaggtggtggaggtggacgagatcga
cgccaagggcctgggcgtggagtacctgtacgacctgaccgtggacatca
accacaactacgtggccaacggcatcgtggtgtccaac

TABLE 17
Pab Klba intein protein, amino acid sequence
(SEQ ID NO: 37).

ALYYFSEIQLPNGKEFIGKLVDELFEKYHDKIGKYKDMEYVELNEEDTFE
VISIGPDLSARRHKVTHVWRRKVKDGEKLVKIRTASGKELVLTQDHPVFV
LLGRDVARRDAGNVKVGDEIAVLNTRPDFSVLSPPAMPELLSEPFNYELS
SIGDVAWDEVVEVDEIDAKGLGVEYLYDLTVDINHNYVANGIVVSN

TABLE 18
Pfu klba intein gene, nucleotide sequence (SEQ
ID NO: 38), native.

gcactttacgatttctctgtcatccaactatctaatggtagatttgtact
tataggagatttagtcgaggaattattcaagaagtatgccgagaaaatta
aaacatacaaagaccttgagtacatagagcttaacgaggaagaccgtttt
gaagttgttagtgttagtccagatttgaaggctaataaacatgttgtctc
aagagtttggagaagaaaggtcagagagggggaaaagctaatacgcataa
agacgagaactggcaacgaaataatcctcactagaaatcatccgctattt
gccttctccaatggagacgtagtcagaaaagaggccgagaagctcaaagt
tggggatagagttgcagtgatgatgagacctccttcacctcctcaaacta
aagctgtagttgaccctgcaatttacgtgaaaataagtgattactacctt
gttccgaacggaaaaggtatgataaaagttcctaacgatggtattcctcc
agaaaaggcccaatatcttctttcagtaaattcatatcctgtaaaattag
tcagagaagttgatgagaagttatcctatctcgctggagttatactcggt
gatgggtatatatcatcgaatggatactacatctcagctacatttgacga
cgaagcttacatggatgcctttgtctctgtagtctcggactttatcccta
actatgtccccagtataaggaagaacggagattacacaattgtaactgtt
ggctcgaagatttttgctgaaatgctctcaaggatatttggaataccaag
gggcagaaaatctatgtgggatattccagacgtagtactttcaaatgacg
atcttatgagatacttcatagctggacttttcgacgctgatgggtacgta
gatgaaaatgggccctccatagtcctagtaacaaagagtgaaaccgtggc
aaggaagatttggtacgttcttcagaggttggggatcataagtacagttt
cccgtgtaaagagcagagggtttaaagaaggcgagctgttcagggtaatt
attagtggtgttgaagatcttgctaaatttgcaaaattcatacccctacg
tcactcaagaaagagggccaaacttatggagatattaaggactaagaagc
catatcggggaagaagaacttaccgcgtgccgatatccagtgatatgata
gctcctctccgtcaaatgttgggattaactgttgcagagctgtctaagtt
agcgtcttattatgcaggggaaaaagtttctgaaagcctaattaggcata
tagaaaagggaagggtcaaagagataagacgctctacgctcaaggggatt
gcccttgctctccagcagatagctaaagatgtgggtaacgaagaagcttg
ggtgagagccaagaggcttcaattgatagctgagggagatgtttactggg
atgaagtcgtaagtgttgaggaagttgatccgaaggagcttggcattgag
tacgtctatgacctcacggttgaggacgaccacaattatgtggcaaatgg
catactagtctcaaac

TABLE 19
Pfu Klba intein protein, amino acid sequence (SEQ
ID NO: 39).

ALYDFSVIQLSNGRFVLIGDLVEELFKKYAEKIKTYKDLEYIELNEEDRF
EVVSVSPDLKANKHVVSRVWRRKVREGEKLIRIKTRTGNEIILTRNHPLF
AFSNGDVVRKEAEKLKVGDRVAVMMRPPSPPQTKAVVDPAIYVKISDYYL
VPNGKGMIKVPNDGIPPEKAQYLLSVNSYPVKLVREVDEKLSYLAGVILG
DGYISSNGYYISATFDDEAYMDAFVSVVSDFIPNYVPSIRKNGDYTIVTV
GSKIFAEMLSRIFGIPRGRKSMWDIPDVVLSNDDLMRYFIAGLFDADGYV
DENGPSIVLVTKSETVARKIWYVLQRLGIISTVSRVKSRGFKEGELFRVI
ISGVEDLAKFAKFIPLRHSRKRAKLMEILRTKKPYRGRRTYRVPISSDMI
APLRQMLGLTVAELSKLASYYAGEKVSESLIRHIEKGRVKEIRRSTLKGI
ALALQQIAKDVGNEEAWVRAKRLQLIAEGDVYWDEVVSVEEVDPKELGIE
YVYDLTVEDDHNYVANGILVSN

We synthesized the nucleotide sequence of the Mja klbA intein and the Pab klbA intein with sequences employing mammalian codon usages. The following mammalian expression vectors are constructed: pTT3-Pab klbA HL(−); pTT3-Pab klbA HL(+); pTT3-Pab klbA LH(−); pTT3-Mja klbA HL(−); pTT3-Mja klbA HL(+); pTT3-Mja klbA LH(−). These constructs were made on the PTT3 vector backbone, as described elsewhere herein. The Pfu klbA intein nucleotide sequence is the native sequence, and pTT3-Pfu-klbA-HL(+) was also constructed.

As indicated for the constructs that use the Lon protease inteins, all the constructs with the “HL” designation have the antibody immunoglobulin heavy chain (HC) coding sequence followed by the intein segment and then by the light chain (LC) coding sequence. Likewise, all the constructs with the “LH” designation have the antibody LC coding sequence followed by the intein segment and then by the HC coding sequence. Constructs with the “(−)” designation have one signal peptide at the beginning of the ORF and a methionine inserted between the last amino acid of the intein segment and the first amino acid of the downstream extein segment, e.g., the mature antibody heavy or light chain that follows the intein. The constructs with the “(+)” designation have one signal peptide at the beginning of the ORF and a second signal peptide at the beginning of the antibody subunit that is down stream of the intein.

The constructs having various KlbA intein segments and configurations were introduced into 293E cells through transient transfection techniques. At seven to eight days post-transfection, the culture supernatants were analyzed for secreted antibody by measuring IgG levels using ELISA. See Table 20 for these results with values in units of micrograms per ml of sample for each construct expression system.

TABLE 20
KlbA intein sORF constructs and secreted antibody production.

| Construct | IgG (μg/ml) |
|---|---|
| pTT3-Pab klbA HL (−) | 19 |
| pTT3-Pab klbA HL (+), | 6 |
| pTT3-Pab klbA LH (−) | 0.4 |
| pTT3-Mja klbA HL (−) | 13 |
| pTT3-Mja klbA HL (+), | 4 |
| pTT3-Mja klbA LH (−) | <0.1 |
| pTT3-Pfu-klbA HL(+) | <0.1 |

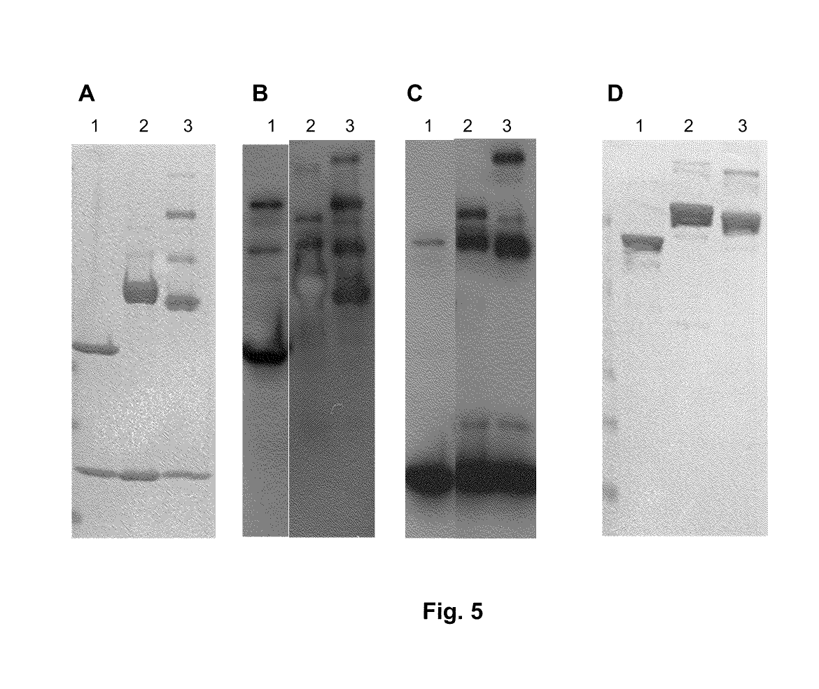

We purified and analyzed the secreted antibody products expressed by the constructs, pTT3-Pab klbA HL(−) and pTT3-Mja klbA HL(−). The antibody products were purified by protein A affinity chromatography and characterized by the electrophoretic technique of SDS-PAGE under both reducing and non-reducing conditions. See FIG. 5. Under non-reducing conditions, culture supernatant samples from these two vectors migrated primarily as a single band, however, with apparently larger molecular weights compared to the control antibody. Under reducing conditions, we found that culture supernatant samples from these two vectors contained detectable bands corresponding in size to the antibody LC and HC-intein fusion components. The corresponding immunoblots using either antibody against IgG1 Fc or kappa light chain are consistent with the characterization of these bands. These results suggest that there is relatively efficient cleavage at the C-terminal splicing junction but less efficient or even little cleavage overall at the N-terminal splicing junction. Even for constructs and expression systems where less than complete cleavage efficiency was achieved, however, the immunoglobulin heavy and light chain subunits were able to assemble and become secreted as complete IgG antibody molecules.

### Modification of Klb Inteins at N-Terminal Intein Splicing Junction

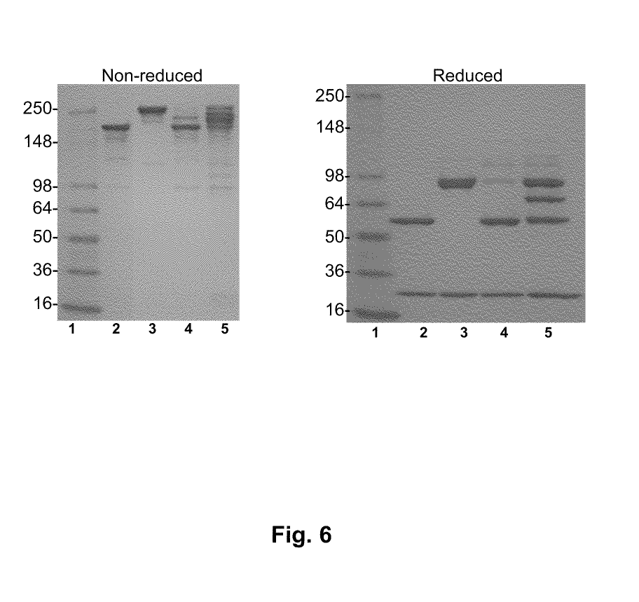

In light of the results described above for cleavage at the N-terminal splicing junction, we engaged in additional efforts to provide for enhanced cleavage efficiency. We noted that the first amino acid of each of these two inteins, Pab klbA and Mja klbA, is alanine (Ala; A) instead of cysteine (Cys; C). We understand that cysteine is a residue capable of functioning as a nucleophile in other intein systems. We tested the effect of reintroducing a nucleophilic amino acid, cysteine, at this position, in combination with introducing one amino acid, glycine, which is native to the klbA extein upstream of the intein, at the end of the immunoglobulin HC segment. See Table 21 which provides sequence information for protein segments for these additional constructs. The table provides the amino acid residues at the two splicing junctions for the native Pab klba intein, the Pab klba HL(−) construct (which is referred to as WT in this context), and three constructs with mutations at the N-terminal splicing junction: Pab klba HL(−)GC; Pab klba HL(−)GA; and Pab klba HL(−)KC. The asterisks (*) indicate positions where variant amino acid residues have been introduced in mutant constructs. Among these constructs, Pab-klbA HL(−)GC demonstrated the ability to express and process proteins with efficient cleavage at the N-terminal intein junction. See also FIG. 6 which illustrates results of expression and SDS-PAGE analysis of IgG proteins from certain constructs.

TABLE 21
Protein sequences for segments of Pab-klbA constructs
with modifications at the N-terminal intein junction.

| | Extein/ | SEQ | | SEQ | | Extein/ | SEQ |
|---|---|---|---|---|---|---|---|
| | HC at | ID | Intein at | ID | Intein at | LC at | ID |
| Construct | C-terminus | NO: | N-terminus | NO: | C-terminus | N-terminus | NO: |
| | * | | * | | | | |
| Pab klbA native | GHD G | 40 | A LYY | 42 | VSN | CMGT | 44 |
| Pab-klbA HL(−) WT | SPG K | 41 | A LYY | 42 | VSN | MDIQ | 45 |
| Pab-klbA HL(−) GC | SP G | | C LYY | 43 | VSN | MDIQ | 45 |
| Pab-klbA HL(−) GA | SP G | | A LYY | 42 | VSN | MDIQ | 45 |
| Pab-klbA HL(−) KC | SPG K | 41 | C LYY | 43 | VSN | MDIQ | 45 |

* Asterisk indicates a position where variation has been introduced.

TABLE 22
Further information for segments of protein sequences in Pab-klbA
constructs.

| | | SEQ ID | N-term. | | |
|---|---|---|---|---|---|
| Construct | HC | NO: | junction | Intein | SEQ ID NO: |
| pTT3-Pab klbA HL (−) | LSLSPGK | 46 | M | ALYYFSEIQ | 48 |
| pTT3-Pab klbA GC | LSLSPG | 47 | C | ALYYFSEIQ | 48 |
| pTT3-Pab klbA GA, | LSLSPG | 47 | — | ALYYFSEIQ | 48 |
| pTT3-Pab klbA KC | LSLSPGK | 46 | C | ALYYFSEIQ | 48 |
| pTT3-Pab klbA KA | LSLSPGK | 46 | — | ALYYFSEIQ | 48 |

### Materials and Methods for Generation of Vectors: Construction of Pab-klbA HL(−) Variants GA, GC, and KC

Several variant forms of certain vector constructs were generated. Pab-klbA HL(−) mutants GA, GC, and KC were constructed by PCR. Forward primer HC-F and reverse primer Hint-R were used to PCR amplify the 3′ end of the heavy chain to generate PCR product #1. Forward primers GA-F, GC-F, and KC-F, contained the desired mutations as well as complementary sequence to the 3′ end of the heavy chain. Primers GA-F, GC-F, or KC-F and the reverse primer Intein-R-2 were used to amplify the 5′ end of the Pab-klbA intein to produce PCR product #2. PCR products #1 and #2 were then purified using a Qiagen Gel Extraction kit. The purified PCR products were annealed together and amplified using the outside primers HC-F and Intein-R-2 to generate PCR product #3. Vector Pab-klbA HL(−) was digested using restriction enzymes SacII and RssII and then purified using a Qiagen Gel Extraction kit. PCR product #3 was subcloned into Pab-klbA-HL(−) (cut with SacII and RssII) by homologous recombination in Maximum Efficiency DH5α cells (Invitrogen). Transformants carrying the correct mutations were determined using colony PCR followed by sequencing. The DNA from correct clones was amplified and purified using a Qiagen Maxi kit. Primer sequences are indicated in the table below.

TABLE 23
Primer sequences for Mutants GA, GC, KC.

| Primer | | SEQ ID |
|---|---|---|
| designation | Nucleic Acid sequence | NO: |
| GA-F | GCCTCTCCCTGTCTCCGGGTGCTCTGTACTACTTCAGCGAGATC | 49 |
| GC-F | GCCTCTCCCTGTCTCCGGGTTGTCTGTACTACTTCAGCGAGATC | 50 |
| KC-F | TCTCCCTGTCTCCGGGTAAATGTCTGTACTACTTCAGCGAGATC | 51 |
| HC-F | CGGCGTGGAGGTGCATAATG | 52 |
| HINT-R | ACCCGGAGACAGGGAGAG | 53 |
| INTEIN-R-2 | GGGTCAGCACCAGTTCTTTG | 54 |

### EXAMPLE 4

### Generation of Stable Vectors and Cell Lines Expressing sORF Constructs

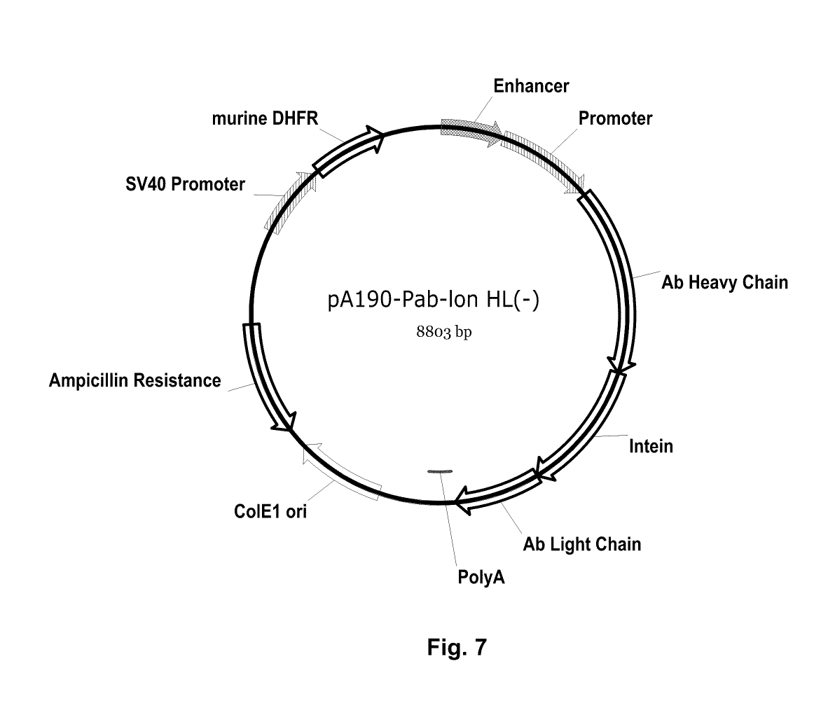

Stable expression vectors and cell lines expressing such vectors can be developed with embodiments of sORF constructs. As an example, a stable expression vector containing a sORF with a Pab Lon intein was stably transfected into a CHO (Chinese hamster ovary) cell line. We designed and prepared a stable sORF expression vector with elements including a CMV enhancer, an adenovirus major late promoter, a SV40 polyA sequence, gastrin transcription terminator, DHFR coding sequence driven by a SV40 promoter, and an ORF which was the same as that in pTT3 pab lon HL(−) which was used in transient expression systems. The sORF construct pA190-Pab-lon HL(−) was thus prepared and is capable of use as a stable expression vector; see FIG. 7. Additional constructs are similarly prepared.

Using the calcium phosphate transfection technique, the pA190 construct was introduced into CHO cells (designated CHO B3.2) which were plated into 48 96-well plates at a density of 200 cells per well in a selection medium containing MEM and 5% FBS. The transfection plates were monitored for growth of cells/colonies and IgG secretion.

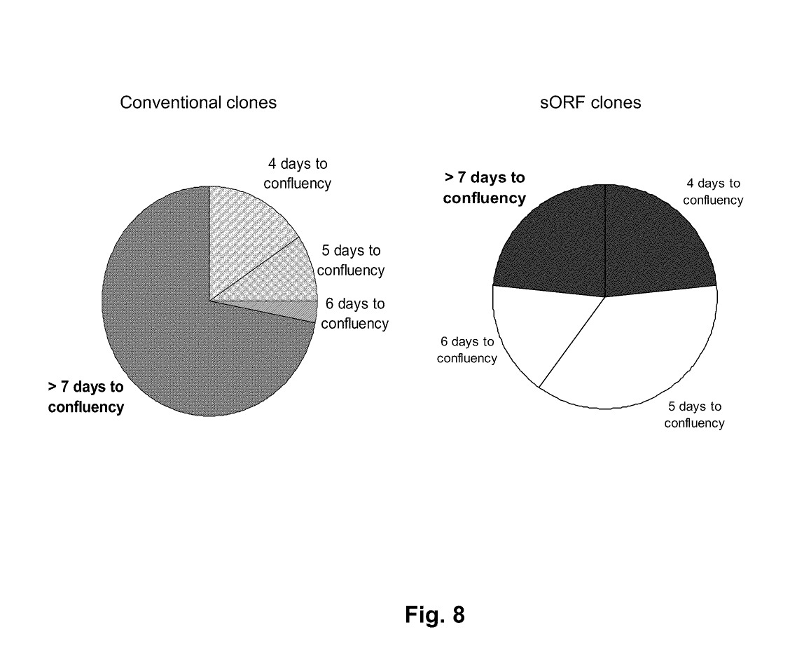

A sampling of 30 clones from sORF stable expression vector pA190-Pab-lon HL(−) and 32 clones from control stable expression vector pA190 transfection reactions were selected and grown. At this stage, the IgG secretion levels were assessed for the selected clones without amplification, and levels were also assessed after amplification with 20 nM methotrexate (MTX). For a stable expression system, the sORF vector generated a significant frequency of growth positive wells (2304 positive out of 4608 total wells) which had an appreciable number (443 of 2304) and rate, 19%, of samples positive for IgG secretion. The IgG secretion levels under conditions of 0 nM MTX in 12-well plates for ˜29 selected clones ranged from about 0.3 to about 2.5 micrograms per ml of culture supernatant. Under conditions with 20 nM MTX, for ˜24 selected clones the IgG secretion levels ranged from about 0.1 to about 6 micrograms per ml, with about half of the clones picked demonstrating secretion levels of greater than 2 μg/ml. It was also noted that the sORF construct clones demonstrated confluency in the adherent culture container relatively rapidly For example, in adherent culture with 20 nM MTX, SORF clones grew faster and reached confluence sooner than the conventional vector clones. At the 1st passage in 20 nM MTX, 28% of clones from the conventional vector reached confluency within 6 days (in either 4 days, 5 days, or 6 days); where as 77% of clones from the sORF pab lon vector reached confluency within 6 days (see FIG. 8). These data suggest a strong advantage of using sORF expression vectors, in comparison to the conventional vectors, in the development of stable expression systems including for CHO cell line development. The sORF clones also demonstrated advantageous levels of antibody secretion under conditions of direct amplification with 100 nM MTX. In an experiment, 16 clones exposed to 100 nM MTX yielded IgG secretion levels averaging 6 ug/ml, with the top five clones averaging 12 ug/ml and the top clone having a production level of 24 ug/ml. The table below shows results of using MTX amplification with the highest expression levels at each amplification step. The values are in micrograms per ml of IgG for various clones having the pA190-Pab-lon-HL(−) construct.

TABLE 24
IgG expression levels with MTX amplification.

| | IgG, ug/ml | | |
|---|---|---|---|
| | | | 100 nM MTX |
| clone # | 0 nM MTX | 20 nM MTX | (direct from 0 nM) |
| 1 | 0.94 | 0.64 | |
| 2 | 0.64 | 0.39 | |
| 3 | 0.75 | | |
| 4 | 0.40 | 0.57 | |
| 5 | 0.83 | 3.90 | |
| 6 | 0.32 | 2.19 | 5.48 |
| 7 | 1.53 | 1.29 | 3.91 |
| 8 | 0.46 | 0.16 | 4.02 |
| 9 | 1.11 | 1.14 | |
| 10 | 0.88 | 2.17 | |
| 11 | 2.50 | 2.23 | |
| 12 | 1.30 | 1.60 | 6.23 |
| 13 | 0.84 | 2.41 | |
| 14 | 1.25 | 1.93 | |
| 15 | 0.86 | 2.68 | 3.78 |
| 16 | 0.50 | 0.30 | 3.51 |
| 17 | 1.05 | 1.13 | 1.42 |
| 18 | 2.23 | 2.45 | |
| 19 | 0.88 | 2.01 | 7.80 |
| 20 | 1.52 | 2.46 | 3.69 |
| 21 | 2.57 | 5.62 | 7.95 |
| 22 | 1.59 | 4.20 | 24.0 |
| 23 | 0.94 | 3.47 | 14.78 |
| 24 | 0.76 | 0.68 | |
| 25 | 1.22 | 4.35 | |
| 26 | 3.67 | 2.62 | 7.56 |
| 27 | 1.29 | 4.26 | 6.96 |
| 28 | 1.29 | 3.14 | 9.71 |
| 29 | 0.96 | | 14.77 |
| 30 | 0.43 | 2.76 | |

### Materials and Methods for Stable Expression Systems.

Chinese hamster ovary cells, cultured in Alpha MEM supplemented with H/T and 10% dialyzed FBS, were transfected with expression vector using a calcium phosphate co-precipitation procedure. See Kingston, R. E., et al. (1993), Unit 16.23: Amplification Using CHO Cell Expression Vectors, Current Protocols in Molecular Biology (Ausubel, F. M., Brent, R., Moore, D. M., Kingston, R. E., Seidman, J. G., Smith, J. A., and Struhl, K., eds; Wiley Interscience, New York), 2:16.23.1. The next day, the cells were transferred using Trypsin/EDTA at room temperature and resuspended in Alpha MEM supplemented with 5% dialyzed FBS (α-MEM+5% dFBS), a growth medium selective for transfected cells expressing DHFR from the expression vector. Culture supernatants that survived the selection were screened using an ELISA specific for human IgG gamma chain. The cell lines that gave the highest ELISA signal were cultured in α-MEM+5% dFBS containing MTX. MTX is an inhibitor of DHFR that selects for cells producing higher levels of the enzyme due to amplification of the vector. Cell lines were cultured in various concentrations of MTX and monitored for the expression of antibody.

In embodiments, compositions and methods of the present invention can employ a pA205 vector construct, or derivative thereof, for example as described in US 20080241883 by Gion et al., Oct. 2, 2008.

Therefore, stable cell expression systems are generated using various sORF designs and constructs. In particular, the sORF systems are well suited for integration with the CHO platform for expression of biological therapeutics such as antibody molecules.

### EXAMPLE 5

### Characterization of Aspects for Intein C-Terminal Splicing Junctions in sORF Constructs

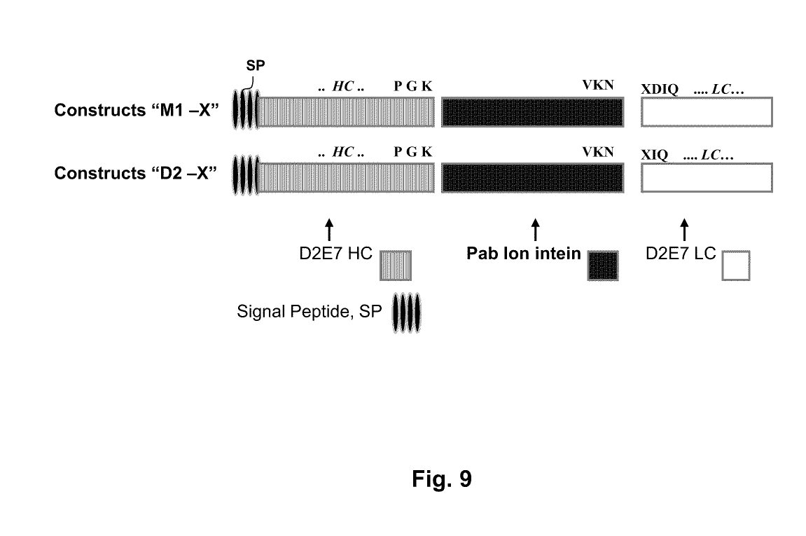

We investigated aspects of the intein C-terminal splicing junction in the context of sORF constructs. We generated about 40 new constructs in part to characterize aspects relating to the first amino acid downstream of the intein and to the splicing junction length which could influence the cleavage efficiency at the C-terminal splicing junction. These further constructs had variations of light chain junction mutations. As an overview, we focused on residues at or near the N-terminal end of the light chain; each of the methionine at position 1 (Met1) and the aspartate at position 2 (Asp2) was replaced with all of the twenty possible natural amino acid residues. See FIG. 9. These two series of light chain junction mutation constructs used the D2E7 antibody coding sequence and the Pab Lon intein segment in the HC-intein-LC configuration.

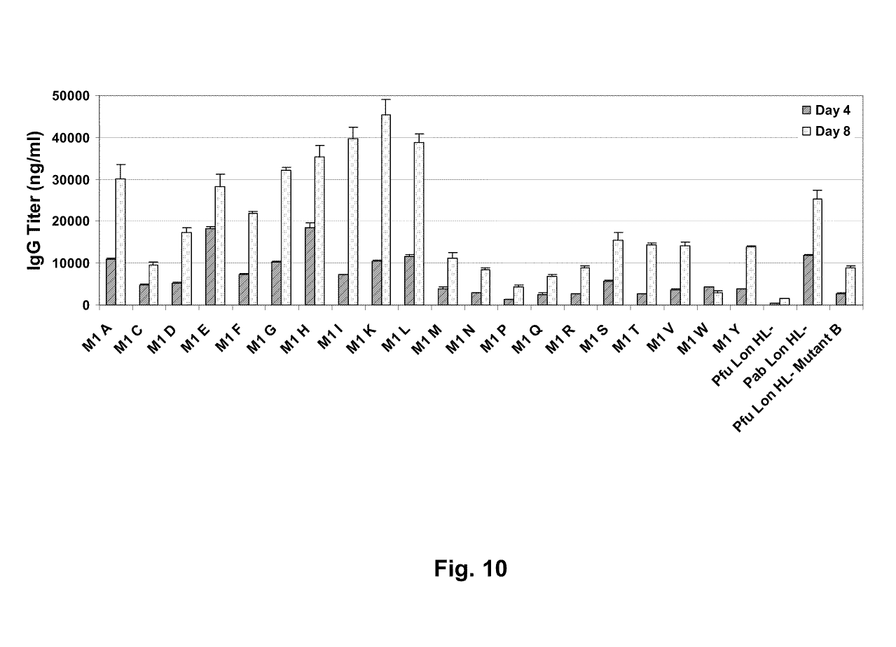

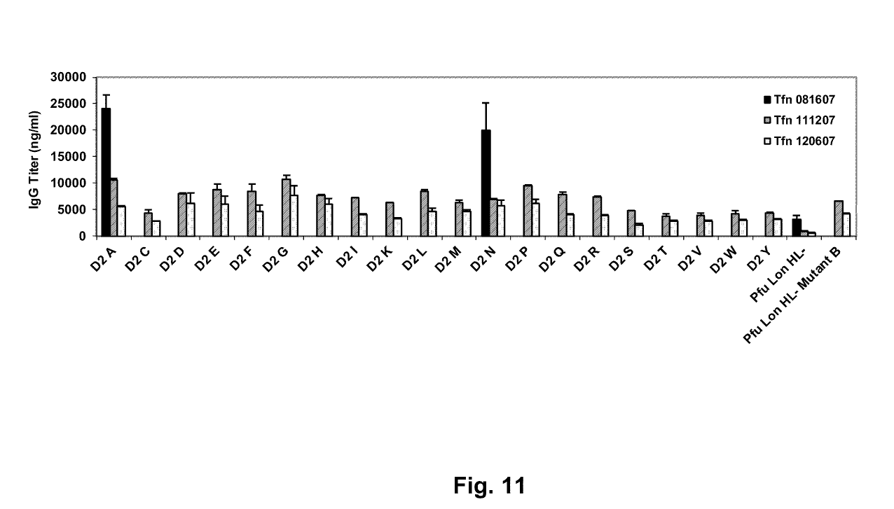

The constructs were transfected into 293 cells. Transient expression yielded high IgG titers using most of the Met substitution constructs, with a number of these constructs yielding expression levels higher than the control construct having Met at this position. See FIG. 10. The Asp substitution constructs generally yielded lower level of antibody expression; see FIG. 11.

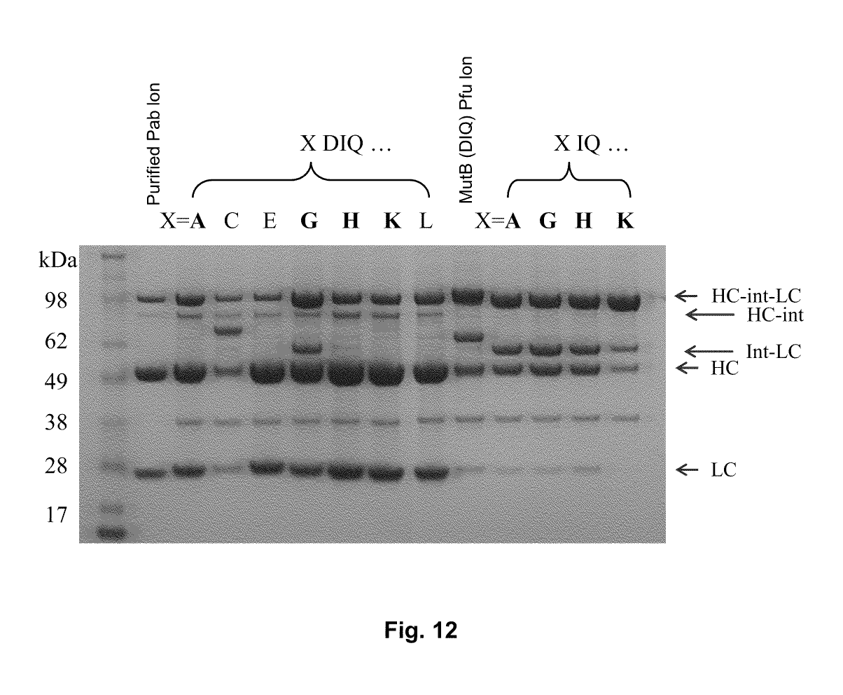

The efficiencies of polyprotein processing were investigated. See, e.g., FIG. 12. The library of constructs based on the variation of Met produced efficient processing of both the HC and the LC from the polyprotein, similar to the Pab Lon HL(−) construct previously described. The library of constructs based on the variation of Asp appeared to have relatively impaired C-terminal processing, generating little LC and significant amounts of intein-LC fusion protein species. This result of incomplete cleavage is interpreted to be independent of the nature of the amino acid at the splicing junction and therefore appears to be associated with the overall length difference of one amino acid unit. It is noted, however, that even constructs which are relatively inefficient can still produce some IgG product.

In an experiment, the IgG antibody products from 10 out of the 20 constructs in the methionine-variation library were further analyzed. Samples were batch purified using Protein A affinity chromatography, and the light chain components were analyzed by mass spectrometry including evaluation of molecular weight. The results of mass spectrometry indicated that antibody light chains from certain constructs (Pab lon M1 A, Pab lon M1 D, Pab lon M1 E, Pab lon M1 F, Pab lon M1 G, Pab lon M1 H, Pab lon M1 I, Pab lon M1 K, Pab lon M1 L, and Pab lon M1 C) were formed as engineered in the construct design with precise cleavage occurring through intein-mediated reactions. The construct in which a Cys was used to replace the Met produced less processed HC and LC components and contained one protein species that could be a splicing product, suggesting that a Cys at the +1 position of the antibody light chain could support protein splicing to some extent. In all the antibody light chains produced, the presence of the first amino acid of the LC demonstrates that the antibody product produced using these vectors will be homogenous in the LC N-terminal region and that the LC are susceptible to processing by amino peptidase activity which can be endogenous.

### EXAMPLE 6

### Expression of Antibody ABT-847 Using sORF Constructs

We developed sORF constructs which were adapted to express antibodies that involve a human lambda light chain. We worked with ABT-847, a fully human antibody with specificity for the antigen, interleukin-12. This antibody has a heavy chain of human IgG1 isotype and a light chain of lambda isotype. See U.S. Pat. No. 6,914,128 by Salfeld, et al., Jul. 5, 2005 for Human antibodies that bind human IL-12 and methods for producing.

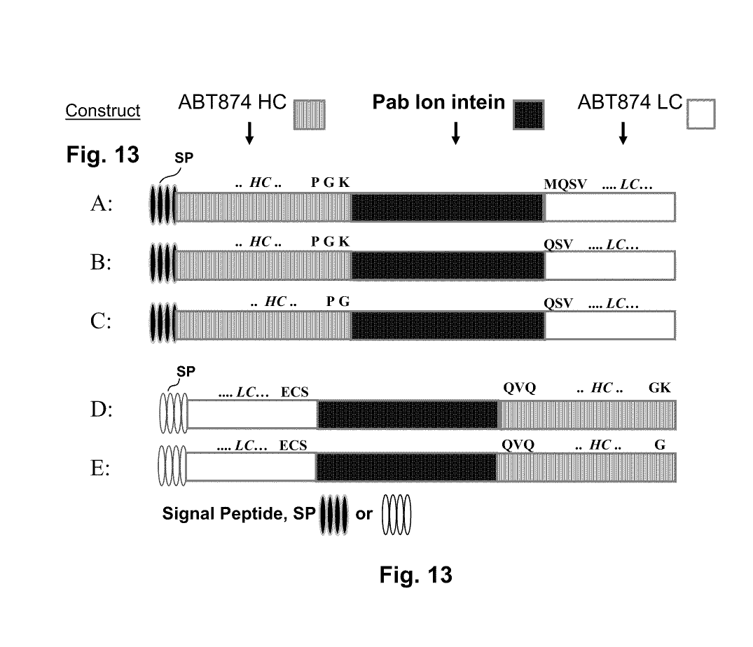

Five sORF constructs capable of expressing ABT-874 were made using homologous recombination. FIG. 13 illustrates structures of the SORF component of these constructs. Three of the constructs had configurations of HC-intein-LC, and two constructs had configurations of LC-intein-HC. These vectors were introduced into HEK293 cells via transient transfection, and their antibody expressions levels were assessed by IgG ELISA. The three HC-intein-LC constructs yielded titers of antibody in samples of culture supernatants which were similar to that of the construct with a similar configuration (HC-intein-LC) but having the D2E7 antibody segments. In both cases, the ABT-874 and D2E7 sORF expression systems employed the Pab Lon HL(−) aspects. The two constructs having the ABT-874 antibody segments in the LC-intein-HC configurations yielded lower levels of IgG titers.

Samples of IgG produced in the supernatant were batch purified by Protein A affinity chromatography and analyzed by SDS-PAGE electrophoresis. These analyses revealed that the LC components were completely processed from the polyprotein in all three of the HC-intein-LC constructs.

The IgG samples were also characterized using mass spectrometry. This analysis confirmed that the LC produced from the three HC-intein-LC constructs started with the appropriate amino acid according to the construct design. This result is consistent with precise cleavage being mediated by the intein used in the configuration. The LC components produced from the two constructs which do not have an extra methionine residue were the same as in material from a control sample of ABT-874 antibody. Thus while modification can be accomplished as was done for the D2E7 antibody, such modification is optional in light of the achievement of a desired N-terminal LC amino acid sequence in the expression product from the described constructs. The mass spectrometry analysis was also used to evaluate the molecular weight of the HC, which demonstrated the expected MW according to the construct design.

### EXAMPLE 7

### sORF Constructs with Intein-Based Purification Tags

In embodiments of the invention, constructs are designed with inserts. In embodiments of constructs with inserts, an insert is capable of providing a detectable signal or is useful in providing a binding or recognition element. In embodiments of the invention, constructs are designed to facilitate the separation of certain construct-related expression products from one or more of such products. For example, vector designs are generated to allow for purification of the fully processed, assembled multimeric antibody product from a mixture of components including partially processed proteins from intein splicing reactions. In the context of expression of an HL construct, the structure of H-intein-L may lead to incomplete cleavage reactions at one or both of the H-intein or intein-L junctions, thus generating protein byproducts of H-intein, intein-L, or the tripartite H-intein-L as opposed to a achievement of completely efficient cleavage which would generate H, intein, and L components. Even in the latter situation, however, it may be useful to remove the intein component. Therefore a strategy was developed to equip an intein with a tag, preferably an internal tag so as to permit at least partial efficiency of the intein cleavage and/or ligation reactions.

As described elsewhere herein, in samples of culture supernatants from sORF constructs we have observed several partially processed intermediates containing the intein protein attached to either the immunoglobulin heavy and/or light chains. We have designed sORF constructs where Pfu lon and Pab lon inteins contain internal polyhistidine tags (IHT). This provides for compositions and methods to allow rapid and efficient separation of unprocessed contaminants.

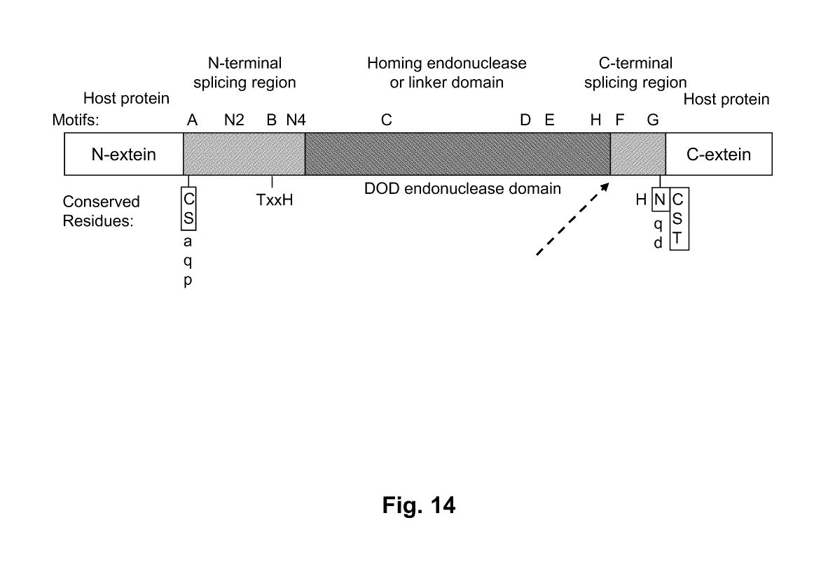

We have found that inteins can be modified by inserting a peptide or a large protein. Preferably, the insertion is made into a solvent accessible loop. By analysis of sequence alignment of several inteins in conjunction with structural modeling, we identified a solvent accessible loop within both the Pyrococcus abyssi (PAB) LON intein and the Pyrococcus furiosus (PFU) LON intein. This loop is located between the endonuclease (H) domain motif and the F/G blocks of an intein (see FIG. 14). FIG. 14 illustrates certain structural motifs of inteins in the context of the location of a preferred location (dashed arrow) of a solvent accessible loop between the H and F/G blocks for introduction of inserts including tags. See also information available at the internet website, http://tools.neb.com/inbase/motifs_endo.php (InBase, The Intein Database: DOD Homing Endonuclease Motifs; InBase Reference: Perler, F. B., 2002, InBase, the Intein Database, Nucleic Acids Res. 30, 383-384) (source of schematic diagram illustrating certain intein structural features including motifs). We have determined that the region between the indicated domains is permissive for many possible insertion sites that can be used within this solvent accessible loop. According to the above source, in FIG. 14 certain features of conserved residues are indicated as follows: boxed amino acids, nucleophiles in standard splicing reaction; uppercase letters, conserved amino acids in standard inteins; lowercase letters, amino acids in polymorphic inteins that may splice by modified mechanisms.

Using site directed mutagenesis, we inserted a polyhistidine affinity tag within this loop and tested the ability of these inteins to function. The IHT does not disrupt the PAB-LON or PFU-LON intein ability to auto-process, and the polyhistidine tag can be used to purify the protein, therefore demonstrating the loop is solvent accessible. In addition, examination of the crystal structure of PFU-RIR1-1 structure suggests that any protein can be inserted in this region as long as it does not substantially or completely disrupt the amino terminal and carboxyl terminal auto-catalytic reaction. For example, polypeptide tags or proteins that have amino and carboxy terminal residues within close proximity should not substantially disrupt the autocatalytic activity of an intein. In preferred embodiments, constructs with inserts such as tags are provided wherein the constructs are capable of exhibiting one or more desired intein activities (e.g., cleavage and/or ligation). Therefore, construct components including this solvent accessible loop of the intein can be modified to create many different functional molecules.

For the Pfu lon intein, tag sequences were inserted. A preferred location for insertion is where the amino acid sequence upstream of the insertion site is IEFIP (AA 323-327) and downstream of the insertion site is ISFSP (AA 328-332). For the Pab lon intein, tag sequences were inserted. A preferred location for insertion is where the amino acid sequence upstream of the insertion site is IIFDA (AA 291-295) and downstream of the insertion site is GRLDV (AA 296-300). In each of the Pfu lon and Pab lon intein constructs, tag sequences which were inserted included the following: HHHHHH (SEQ ID NO:56), HHHHHHHHHH (SEQ ID NO:57), and HQHQHQ (SEQ ID NO:58). As demonstrated by the latter tag sequence (where H=histidine and Q=glutamine), inserts can be other than polyhistidine tags. Further insert sequences can be used as would be understood in the art.

The IHT-modified intein sORF constructs yielded levels of antibody secretion similar to those without the IHT modification. This result suggests that the IHT modification does not disrupt the ability of the intein to function in its auto-processing of the sORF product, nor does the IHT prevent the correctly processed antibodies from being secreted into the media.

Using immobilized metal affinity chromatography, we demonstrated that the intein containing contaminants can be rapidly and efficiently removed from protein A purified antibody preparations via the IHT. These IHT constructs have enabled us to separate correctly processed antibodies and represent a complementary method for the production of sORF-derived biologicals including therapeutic antibodies.

Using internally His-tagged sORF constructs, the D2E7 antibody molecules were efficiently separated from the intein-containing protein species in flow through mode with the technique of nickel column chromatography. Similarly, D2E7 antibody produced using the Pab lon HL(−) construct was also efficiently separated from the intein-containing protein species using Q-column. The IgG samples produced from the Pab lon HL(−) purified by protein A technology, and the IgG samples produced from the Pab lon HL(−)/10 His constructs and purified by both proA and Ni-resins were also analyzed by SEC fractionation. The post-purification samples showed improved purity of monomeric IgG species. Size exclusion chromatography (SEC) further removed residual minor contaminants. The purified IgG samples were analyzed by BiaCore for binding affinity to the specific antigen, TNFa. The affinities of these samples were indistinguishable from the D2E7 antibody produced using conventional vector.

### EXAMPLE 8

### Pho Ion Intein

The amino acid sequence for the Pho lon intein of Pyrococcus horikoshii OT3 is indicated below. See also Accession No. BAA29538.1 in NCBI/protein, PH0452, according to Inbase, the NEB Intein Database.

TABLE 25
Pho Ion, amino acid sequence (SEQ ID NO: 55)

QCFSGEEVIIVEKGKDRKVVKLREFVEDALKEPSGEGMDGDIKVTYKDLR
GEDVRILTKDGFVKLLYVNKREGKQKLRKIVNLDKDYWLAVTPDHKVFTS
EGLKEAGEITEKDEIIRVPLVILDGPKIASTYGEDGKFDDYIRWKKYYEK
TGNGYKRAAKELNIKESTLRWWTQGAKPNSLKMIEELEKLNLLPLTSEDS
RLEKVAIILGALFSDGNIDRNFNTLSFISSERKAIERFVETLKELFGEFN
YEIRDNHESLGKSILFRTWDRRIIRFFVALGAPVGNKTKVKLELPWWIKL
KPSLFLAFMDGLYSGDGSVPRFARYEEGIKFNGTFEIAQLTDDVEKKLPF
FEEIAWYLSFFGIKAKVRVDKTGDKYKVRLIFSQSIDNVLNFLEFIPISL
SPAKREKFLREVESYLAAVPESSLAGRIEELREHFNRIKKGERRSFIETW
EVVNVTYNVTTETGNLLANGLFVKNS

### EXAMPLE 9

### Vectors and Light Chain Signal Peptides

Further advances have been made in the context of single open reading frame vectors for expression of proteins. In embodiments, the vectors employ proteins of immunoglogulins for expression in mammalian cells. In such vectors, configurations of components of light chain components including light chain signal peptides are designed and generated.

In embodiments of single open reading frame constructs, signal peptides from native human antibody light chain sources are employed. For example, human light chain signal peptides are reported in V BASE, which includes a database of information on human germline variable region sequences from sources such as Genbank and EMBL data libraries. The V BASE database is affiliated with the MRC Centre for Protein Engineering, Cambridge, United Kingdom (available at internet address, http://vbase.mrc-cpe.cam.ac.uk/). See also Giudicelli V et al., Nucleic Acids Research, 2006, Vol. 34, Database issue D781-D784; Retter I et al., Nucleic Acids Res. 2005 Jan. 1; 33(Database Issue): D671-D674. In particular embodiments, multiple vector designs are provided which can be used to produce IgG1 antibody with various amino acids including natural amino acids.

Certain human light chain signal peptides are provided which generally range in length of 19-23 amino acids and have sequences as indicated in the table(s) below (Hu Vk, human kappa variable region; LCSP, light chain signal peptide). Variant peptides are also provided which can vary in amino acid sequence and/or length relative to native peptides including such of human origin. For a given amino acid sequence, a gap may be indicated for the purpose of comparison relative to an alignment with one or more other sequences. In an embodiment, a given light chain signal peptide or nucleic acid sequence encoding therefor is provided. In an embodiment, an amino acid or nucleic acid sequence is provided as a synthetic construct of the sequence or segment thereof such as in an expression vector, a synthesized (such as chemically synthesized) molecule, or a recombinant expression product.

TABLE 26
VK Leader Sequences, Part 1

| Human | LCSP | | SEQ ID |
|---|---|---|---|
| Vk | Item | Amino Acid Sequence | NO: |
| — | — | −20 −10 −1 | — |
| — | — | \| \| \| | — |
| VKI | O12 | MDMRVPAQLLGLLLLWLRGARC | 59 |
| | O2 | MDMRVPAQLLGLLLLWLRGARC | 60 |
| | O18 | MDMRVPAQLLGLLQLWLSGARC | 61 |
| | O8 | MDMRVPAQLLGLLLLWLSGARC | 62 |
| | A20 | MDMRVPAQLLGLLLLWLPDTRC | 63 |
| | A30 | MDMRVPAQLLGLLLLWFPGARC | 64 |
| | L14 | MDMRVPAQLLGLLLLWFPGARC | 65 |
| | L1 | MDMRVLAQLLGLLLLCFPGARC | 66 |
| | L15 | MDMRVLAQLLGLLLLCFPGARC | 67 |
| | L4 | MDMRVPAQLLGLLLLWLPGARC | 68 |
| | L18 | MDMRVPAQLLGLLLLWLPGARC | 69 |
| | L5 | MDMRVPAQLLGLLLLWFPGSRC | 70 |
| | L19 | MDMRVPAQLLGLLLLWFPGSRC | 71 |
| | L8 | MDMRVPAQLLGLLLLWLPGARC | 72 |
| | L23 | MDMRVPAQRLGLLLLWFPGARC | 73 |
| | L9 | MRVPAQLLGLLLLWLPGARC | 74 |
| | L24 | MDMRVPAQLLGLLLLWLPGARC | 75 |
| | L11 | MDMRVPAQLLGLLLLWLPGARC | 76 |
| | L12 | MDMRVPAQLLGLLLLWLPGAKC | 77 |
| VKII | O11 | MRLPAQLLGLLMLWVPGSSE | 78 |
| | O1 | MRLPAQLLGLLMLWVPGSSE | 79 |
| | A17 | MRLPAQLLGLLMLWVPGSSG | 80 |
| | A1 | MRLPAQLLGLLMLWVPGSSG | 81 |
| | A18 | MRLPAQLLGLLMLWIPGSSA | 82 |
| | A2 | MRLPAQLLGLLMLWIPGSSA | 83 |
| | A19 | MRLPAQLLGLLMLWVSGSSG | 84 |
| | A3 | MRLPAQLLGLLMLWVSGSSG | 85 |
| | A23 | MRLLAQLLGLLMLWVPGSSG | 86 |
| VKIII | A27 | METPAQLLFLLLLWLPDTTG | 87 |
| | A11 | METPAQLLFLLLLWLPDTTG | 88 |
| | L2 | MEAPAQLLFLLLLWLPDTTG | 89 |
| | L16 | MEAPAQLLFLLLLWLPDTTG | 90 |
| | L6 | MEAPAQLLFLLLLWLPDTTG | 91 |
| | L20 | MEAPAQLLFLLLLWLTDTTG | 92 |
| | L25 | MEPWKPQHSFFFLLLLWLPDTTG | 93 |
| VKIV | B3 | MVLQTQVFISLLLWISGAYG | 94 |
| VKV | B2 | MGSQVHLLSFLLLWISDTRA | 95 |
| VKVI | A26 | MLPSQLIGFLLLWVPASRG | 96 |
| | A10 | MLPSQLIGFLLLWVPASRG | 97 |
| | A14 | MVSPLQFLRLLLLWVPASRG | 98 |

TABLE 27
VK Leader Sequences, Part 2

| Human | LCSP | | SEQ ID |
|---|---|---|---|
| Vk | Item | Amino Acid Sequence | NO: |
| — | — | −20 −10 −1 | — |
| — | — | \| \| \| | — |
| VKI | L5 | MDMRVPAQLLGLLLLWFPGSRC | 70 |
| Mutant | 2G | MDMRVPAQLLGLLLLWFPGSGG | 99 |
| | 3G | MDMRVPAQLLGLLLLWFPGSGGG | 100 |
| | 4G | MDMRVPAQLLGLLLLWFPGSGGGG | 101 |
| | 5G | MDMRVPAQLLGLLLLWFPGSGGGGG | 102 |
| | 1R | MRMRVPAQLLGLLLLWFPGSRC | 103 |
| | 1R2G | MRMRVPAQLLGLLLLWFPGSGG | 104 |
| | 2R | MRRMRVPAQLLGLLLLWFPGSRC | 105 |
| | 2R2G | MRRMRVPAQLLGLLLLWFPGSGG | 106 |
| | 3R2G | MRRRMRVPAQLLGLLLLWFPGSGG | 107 |
| | H2G | MDMRVPAQLLG DEWFPGSGG | 108 |
| | — | — | — |

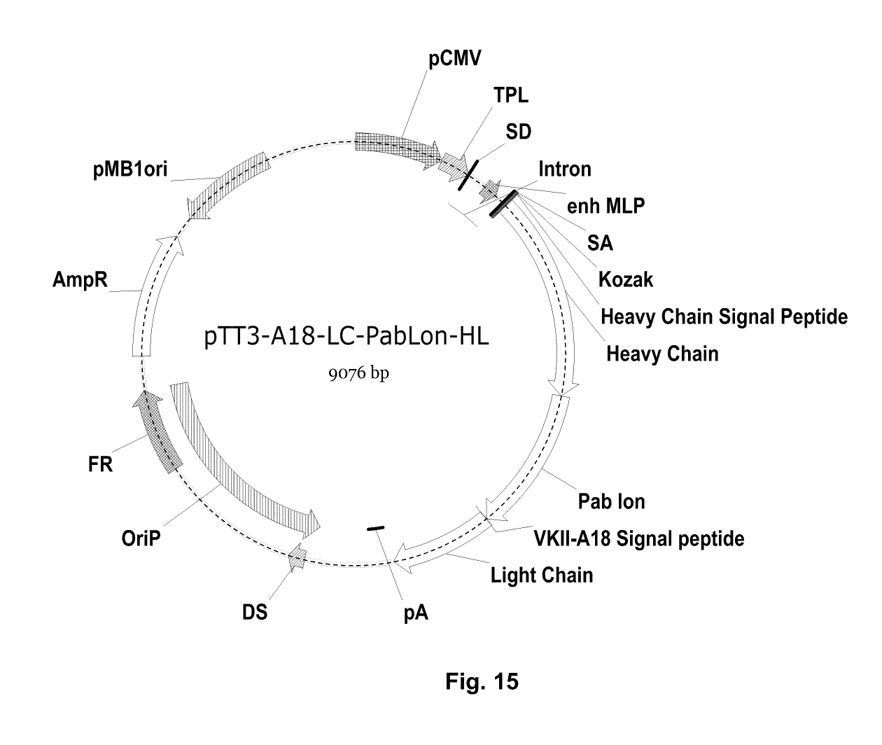

Certain Vkappa signal peptides were substituted for L5, which is the signal peptide for the immunoglobulin light chain designated E7 (corresponding to the light chain of the antibody molecule D2E7 which has antigen specificity for tumor necrosis factor alpha). Also, a mutation library of certain mutants of L5 was constructed, and mutant L5 peptides were substituted for the native L5 peptide. Mammalian expression vectors were constructed in the HL orientation using the Pyrococcus abyssi lon intein. The following vectors were generated: pTT3-A14-E7-PablonHL, pTT3-A17-E7-PablonHL, pTT3-A18-E7-PablonHL, pTT3-A19-E7-PablonHL, pTT3-A23-E7-PablonHL, pTT3-A26-E7-PablonHL, pTT3-A27-E7-PablonHL, pTT3-B2-E7-PablonHL, pTT3-B3-E7-PablonHL, pTT3-L2-E7-PablonHL, pTT3-L20-E7-PablonHL, pTT3-L25-E7-PablonHL, pTT3-mut-1R-E7-PablonHL, pTT3-mut-1R2G-E7-PablonHL, pTT3-mut-2R-E7-PablonHL, pTT3-mut-2G-E7-PablonHL, pTT3-mut-2R2G-E7-PablonHL, pTT3-mut-3G-E7-PablonHL, pTT3-mut-3R2G-E7-PablonHL, pTT3-mut-4G-E7-PablonHL, pTT3-mut-H+2G-E7-PablonHL. These constructs were made on the PTT3 vector backbone. This vector has EBV origin of replication, which allows for its episomal amplification in tranfected 293E cells (cells that express Epstein-Barr virus nuclear antigen 1) in suspension culture. Each vector had one ORF, driven by a CMV promoter. In the ORF, the intein sequence was inserted in frame between the antibody heavy and light chains, in the order of HC-intein-LC. A schematic diagram of the construct structure for pTT3-A18-E7-PablonHL is shown in FIG. 15.

The constructs were introduced into 293E cells through transient transfection, and multiple transient expression experiments were performed. In a given experiment, samples were collected from the supernatant of cultures of the transfected cells on the eighth day post-transfection and analyzed. The samples contained levels of secreted antibody as assessed by IgG ELISA, for which data is shown in the table below in terms of micrograms of antibody per milliliter of sample. The native control was a vector which used the L5 LCSP sequence. As another control (not shown in table), a conventional two-vector system expressing the same antibody, and using the same regulatory elements, was included with these experiments; the antibody secretion level produced from this control vector system ranged from 80 to 206 μg/ml. The IgG secretion level produced by several of the sORF construct designs using these light chain signal peptides are comparable to the order of the range produced using the conventional vector. These expression levels are significantly higher than that using the native L5 E7 signal peptide (2.0 μg/ml using the Pablon HL(+) construct). These antibody secretion levels are also significantly higher than that produced using the “2A” technology, which was reported to be at 1.6 ug/ml in mammalian cells (Fang et al., 2005, Nature Biotechnology 23:584-590).

TABLE 28
Antibody Levels from LCSP Constructs.

| | LCSP Vector | IgG, |
|---|---|---|
| Item | Component | μg/ml |
| 1 | native control (L5) | 2.15 |
| 2 | A14 | 7.25 |
| 3 | A17 | 56.85 |
| 4 | A18 | 41.9 |
| 5 | A19 | 15.7 |
| 6 | A23 | 3.25 |
| 7 | A26 | 27.5 |
| 8 | A27 | 4.6 |
| 9 | B2 | 9.1 |
| 10 | B3 | 1.7 |
| 11 | L2 | 6.65 |
| 12 | L20 | 1.9 |
| 13 | L25 | 0.15 |
| 14 | 2G | 9.25 |
| 15 | 3G | 1.9 |
| 16 | 4G | 3.05 |
| 17 | 5G | 4.25 |
| 18 | 1R | 0.5 |
| 19 | 1R2G | 4.25 |
| 20 | 2R | 0.1 |
| 21 | 2R2G | 1.55 |
| 22 | 3R2G | 1.2 |
| 23 | H2G | 99.3 |

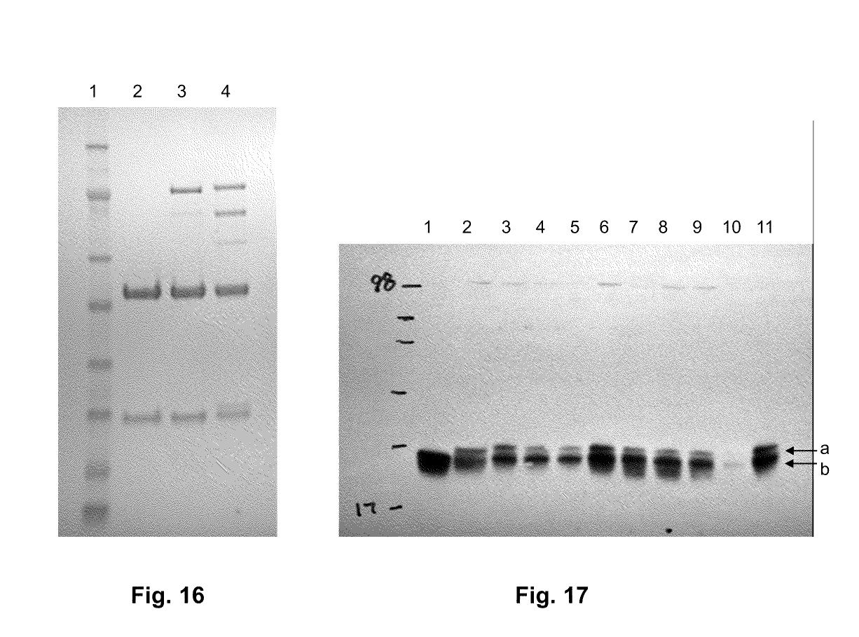

Of the indicated constructs for which antibody product levels were measured, products from five of the constructs which produced the highest levels of secreted antibody were selected for further analysis. The products corresponded to the following five constructs: pTT3-A17-E7-PablonHL, pTT3-A18-E7-PablonHL, pTT3-A19-E7-PablonHL, pTT3-A26-E7-PablonHL, and pTT3-mutH+2G-E7-PablonHL. The secreted antibody produced from these constructs was purified by protein A affinity chromatography and analyzed on reducing SDS-PAGE gels, and the N-terminal amino acid sequences for their HC and LC were determined. The samples produced using pTT3-A18-E7-PablonHL contained protein migration bands corresponding to the antibody heavy and light chains with migrations indistinguishable from a similar antibody produced by traditional methods. On reducing gels, in addition to the bands corresponding to the antibody HC and LC, there were also two higher molecular weight bands that appear to correspond to the unprocessed tripartite protein (HC-intein-LC). Such construct-related contaminants as unprocessed or partially processed proteins can be conveniently removed as described herein and according to conventional techniques. See FIG. 16 which depicts an example of results from an SDS-PAGE analysis. The secreted IgG antibodies were purified by Protein A affinity chromatography and separated using the technique of SDS-PAGE in a gel under reducing conditions. Samples in lanes from left to right are: Lane (1) SeeBlue Plus2 Protein Standard (Invitrogen) protein molecular weight markers; (2) control, D2E7 antibody produced with a traditional non-sORF expression vector system; (3) Pab-lon HL(−); (4) pTT3-A18-E7-PablonHL.

Intracellular samples of products of expression constructs were also analyzed by western blot analysis using antibodies against both HC and LC. Similar protein species were observed as in the cultured supernatant. The N-terminal amino acid sequences of both heavy chain and light chains were determined to be native by mass spectrometry analysis. The analysis confirmed that the A18 signal peptide cleavage had taken place at precisely the correct point which was desired for expression of the light chain. In addition, similarity to traditionally expressed D2E7 was demonstrated using Cation Exchange Chromatography (CIEX), which separates proteins based on net surface charge and which is capable of detecting variants of D2E7 and impurities. Therefore, the A18 signal peptide can be employed in sORF vectors for antibody expression and is capable of efficiently expressing a fully processed and assembled antibody product.

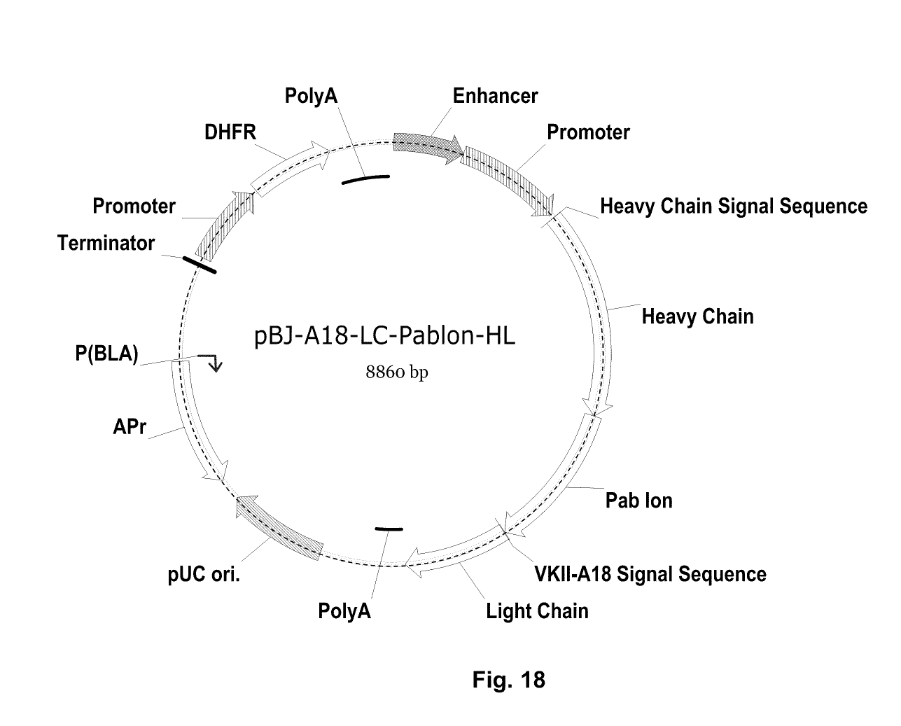

In addition to the transient expression systems describe above, stable cell lines were also generated for expression of antibody products. Stable CHO cell lines were made with the sORF expression constructs using vectors having the A18 light chain signal peptide component. The sORF construct A18-E7-PablonHL was cloned into plasmid pA190 using recombinant techniques. See FIG. 18 which provides a schematic diagram of the construct structure for pBJ-A18-LC-Pablon-HL (also referred to as pA190-A18-E7-PablonHL). This construct was transfected by the calcium phosphate method into CHO B3.2 cells and plated into 48 96-well plates in Minimum Essential Medium (MEM) Alpha Medium with 5% FBS. Transfection samples were screened and subjected to amplification methods with up to 100 nM MTX. The results of expression of the construct in cultures were characterized. At 100 nM MTX, cells with the construct pA190-A18-E7-PablonHL expressed antibody in the range of 1.1 to 16.9 μg/ml in samples of culture supernatant. The four highest expressing clones were subcloned by limiting dilution. The subclones were tested and found to express antibody in amounts of 2.9 to 31.8 μg/ml. Four of the subcloned cell lines with the highest expression levels were adapted to grow in suspension and produced average amounts of 31 to 44 μg/ml as measured from samples taken at day four of culture.

The ability of the constructs to generate mature light chain products was assessed. See FIG. 17 which provides results from a Western blot experiment. Samples of intracellular antibody products were characterized. Whole cell lysates were separated according to SDS-PAGE in a gel under reducing conditions, transferred to nitrocellulose membranes, blocked with blocking solution (nonfat dry milk in TTBS, tris-buffered saline with Tween 20), incubated with horseradish peroxidase-conjugated antibodies to either heavy or light chain and developed using ECL (enhanced chemiluminescence) reagent. Samples according to construct designations in lanes from left to right in the blot are: unnumbered lane at left, molecular weight markers; (Lane 1) control, D2E7 from CHO cells; (2) control, pTT3-A18-E7-PablonHL in transient transfection with HEK293 cells; (3-11) various clones from pA190-A18-E7-PablonHL, corresponding respectively to clone numbers 1, 3, 7, 9, 12, 14, 18, 15, and 13 respectively. Arrows labeled with letters “a” and “b” indicate expression products as follows: (a) upper band, light chain with signal peptide, and (b) lower band, mature light chain. In the mature light chain product, the signal peptide has been cleaved, resulting in a product with lower molecular weight relative to the precursor. The results demonstrate that constructs with the A18 light chain signal peptide component are able to express and produce the fully mature light chain product which is comparable to that of the D2E7 antibody.

### STATEMENTS REGARDING INCORPORATION BY REFERENCE AND VARIATIONS

Any sequence listing information is considered part of the specification.

All references mentioned throughout this application, for example patent documents including issued or granted patents or equivalents; patent application publications; unpublished patent applications; and non-patent literature documents or other source material; are hereby incorporated by reference herein in their entireties, as though individually incorporated by reference. In the event of any inconsistency between cited references and the disclosure of the present application, the disclosure herein takes precedence. Some references provided herein are incorporated by reference to provide information, e.g., details concerning sources of starting materials, additional starting materials, additional reagents, additional methods of synthesis, additional methods of analysis, additional biological materials, additional cells, and additional uses of the invention.

All patents and publications mentioned herein are indicative of the levels of skill of those skilled in the art to which the invention pertains. References cited herein can indicate the state of the art as of their publication or filing date, and it is intended that this information can be employed herein, if needed, to exclude specific embodiments that are in the prior art. For example, when composition of matter are claimed herein, it should be understood that compounds known and available in the art prior to Applicant's invention, including compounds for which an enabling disclosure is provided in the references cited herein, are not intended to be included in the composition of matter claims herein.

Any appendix or appendices hereto are incorporated by reference as part of the specification and/or drawings.

When a compound, construct or composition is claimed, it should be understood that compounds, constructs and compositions known in the art including those taught in the references disclosed herein are not intended to be included. When a Markush group or other grouping is used herein, all individual members of the group and all combinations and subcombinations possible from within the group the group are intended also to be individually set forth and included in the disclosure.

Where the terms “comprise”, “comprises”, “comprised”, or “comprising” are used herein, they are to be interpreted as specifying the presence of the stated features, integers, steps, or components referred to, but not to preclude the presence or addition of one or more other feature, integer, step, component, or group thereof. Thus as used herein, comprising is synonymous with including, containing, having, or characterized by, and is inclusive or open-ended and does not exclude additional, unrecited elements or method steps. As used herein, “consisting of” excludes any element, step, or ingredient, etc. not specified in the claim description. As used herein, “consisting essentially of” does not exclude materials or steps that do not materially affect the basic and novel characteristics of the claim (e.g., relating to an active ingredient). In each instance herein any of the terms “comprising”, “consisting essentially of” and “consisting of” may be replaced with at least either of the other two terms, thereby disclosing separate embodiments and/or scopes which are not necessarily coextensive. An embodiment of the invention illustratively described herein suitably may be practiced in the absence of any element or elements or limitation or limitations not specifically disclosed herein.

Whenever a range is disclosed herein, e.g., a temperature range, time range, composition or concentration range, or other value range, etc., all intermediate ranges and subranges as well as all individual values included in the ranges given are intended to be included in the disclosure. This invention is not to be limited by the embodiments disclosed, including any shown in the drawings or exemplified in the specification, which are given by way of example or illustration and not of limitation. It will be understood that any subranges or individual values in a range or subrange that are included in the description herein can be excluded from the claims herein.

The invention has been described with reference to various specific and/or preferred embodiments and techniques. However, it should be understood that many variations and modifications may be made while remaining within the spirit and scope of the invention. It will be apparent to one of ordinary skill in the art that compositions, methods, devices, device elements, materials, procedures and techniques other than those specifically described herein can be employed in the practice of the invention as broadly disclosed herein without resort to undue experimentation; this can extend, for example, to starting materials, biological materials, reagents, synthetic methods, purification methods, analytical methods, assay methods, and biological methods other than those specifically exemplified. All art-known functional equivalents of the foregoing (e.g., compositions, methods, devices, device elements, materials, procedures and techniques, etc.) described herein are intended to be encompassed by this invention. The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by embodiments, preferred embodiments, and optional features, modification and variation of the concepts herein disclosed may be resorted to by those skilled in the art, and that such modifications and variations are considered to be within the scope of this invention as defined by the appended claims.

### REFERENCES

This application incorporates by reference in particular each of the following items in entirety: U.S. Provisional Patent Application Ser. 61/256,544 filed Oct. 30, 2009 by Gerald R. Carson et al.; U.S. patent application Ser. No. 12/822,598 filed Jun. 24, 2010 by Gerald R. Carson et al.; U.S. patent application Ser. No. 11/459,098 filed Jul. 21, 2006 by Gerald R. Carson et al. (published as US 20070065912, Mar. 22, 2007); U.S. Provisional Patent Application Ser. No. 60/701,855 filed Jul. 21, 2005 by Gerald R. Carson et al.; and PCT International Application No. PCT/US06/28691 filed Jul. 21, 2006 by Gerald R. Carson et al. (published as WO/2007/014162, Feb. 1, 2007).

US Patent Documents: U.S. Pat. No. 5,981,182 by Jacobs, Jr., et al., Nov. 9, 1999; U.S. Pat. Nos. 7,105,341; 7,378,248 by Lorens, et al., May 27, 2008; U.S. Pat. No. 6,933,362 by Belfort, et al., Aug. 23, 2005. U.S. Pat. No. 6,090,382 by Salfeld, et al. issued Jul. 18, 2000 for Human antibodies that bind human TNF.alpha. U.S. Pat. No. 6,914,128 by Salfeld, et al., Jul. 5, 2005 for Human antibodies that bind human IL-12 and methods for producing. U.S. Pat. No. 6,258,562 by Salfeld, et al., Jul. 10, 2001 for Human antibodies that bind human TNF.alpha.

US Patent Documents: 20030036643 A1 by Jin, Cheng He; et al., published Feb. 20, 2003; 20050158820 A1 by Kinsella, Todd M., published Jul. 21, 2005 for In vivo production of cyclic peptides. US Patent Application Publication 20050147610 by Ghayur, Tariq et al., Jul. 7, 2005. U.S. Pat. No. 5,756,095 by Jutila, May 26, 1998 for Antibodies with specificity for a common epitope on E-selectin and L-selectin.

US 20070081996 by Hoffman; Rebecca S.; et al. Apr. 12, 2007 for Method of treating depression using a TNFalpha antibody.

Chen L, Benner J, Perler F B., Protein splicing in the absence of an intein penultimate histidine. J Biol Chem. 2000 Jul. 7; 275(27):20431-5.

Cohen G N, et al., An integrated analysis of the genome of the hyperthermophilic archaeon Pyrococcus abyssi. Mol Microbiol. 2003 March; 47(6):1495-512.

Durocher Y, Perret S, Kamen A., Nucleic Acids Res. 2002 Jan. 15; 30(2):E9, High-level and high-throughput recombinant protein production by transient transfection of suspension-growing human 293-EBNA1 cells.

Fukui T, Eguchi T, Atomi H, Imanaka T. A membrane-bound archaeal Lon protease displays ATP-independent proteolytic activity towards unfolded proteins and ATP-dependent activity for folded proteins. J Bacteriol. 2002 July; 184(13):3689-98. PMID: 12057965.

Gandor C, et al., 1995 FEBS Letters 377:290-294.

Goddard M R, Burt A. Recurrent invasion and extinction of a selfish gene. Proc Natl Acad Sci USA. 1999 Nov. 23; 96(24):13880-5.

Gogarten J P, Hilario E. Inteins, introns, and homing endonucleases: recent revelations about the life cycle of parasitic genetic elements. BMC Evol Biol. 2006 Nov. 13; 6:94. PMID: 17101053

Gogarten J P, Senejani A G, Zhaxybayeva O, Olendzenski L, Hilario E. Inteins: structure, function, and evolution. Annu Rev Microbiol. 2002; 56:263-87.

International Publication No.: WO/2005/086654 for International Application No.: PCT/US2005/005763, Publication Date: Sep. 22, 2005 by Wood David W et al.

Kimball A B, et al., Arch Dermatol. 2008 February; 144(2):200-7, Safety and efficacy of ABT-874, a fully human interleukin 12/23 monoclonal antibody, in the treatment of moderate to severe chronic plaque psoriasis: results of a randomized, placebo-controlled, phase 2 trial.

Liao Y D, Jeng J C, Wang C F, Wang S C, Chang S T. Removal of N-terminal methionine from recombinant proteins by engineered E. coli methionine aminopeptidase. Protein Sci. 2004 July; 13(7):1802-10.

Lecompte, O.; Ripp, R.; Puzos-Barbe, V.; Duprat, S.; Heilig, R.; Dietrich, J.; Thierry, J. C.; Poch, O. (2001) Genome evolution at the genus level: comparison of three complete genomes of hyperthermophilic archaea. Genome Res. 11(6): 981-93. PubMed ID: 11381026.

Mills Kenneth V., Jennifer S. Manning, Alicia M. Garcia, and Lisa A. Wuerdeman, Protein Splicing of a Pyrococcus abyssi Intein with a C-terminal Glutamine, The Journal of Biological Chemistry, Vol. 279, No. 20, Issue of May 14, pp. 20685-20691, 2004.

Mills Kenneth V., Deirdre M. Dorval, and Katherine T. Lewandowski, Kinetic Analysis of the Individual Steps of Protein Splicing for the Pyrococcus abyssi PolII Intein, The Journal of Biological Chemistry, Vol. 280, No. 4, Issue of January 28, pp. 2714-2720, 2005.

Powell K T and Weaver J C, 1990 Bio/Technology 8:333-337.

Saves I, Morlot C, Thion L, Rolland J L, Dietrich J, Masson J M, Nucleic Acids Res. 2002 Oct. 1; 30(19):4158-65. Investigating the endonuclease activity of four Pyrococcus abyssi inteins.

Senejani A G, Hilario E, Gogarten J P. The intein of the Thermoplasma A-ATPase A subunit: structure, evolution and expression in E. coli. BMC Biochem. 2001; 2:13. PMID: 11722801

Southworth M W, Benner J, Perler F B, EMBO J. 2000; 19(18):5019-26. An alternative protein splicing mechanism for inteins lacking an N-terminal nucleophile.

Xie, J.; Juang, J. F.; Shi, X. F.; Liu, C. Q. (2001) Analysis of the characteristic sequence of intein and revision of its motifs. Chinese Sci Bull 46: 758-761.

Mannon P J et al., 2004, N Engl J Med. 2004; 351(20):2069-79, Anti-interleukin-12 antibody for active Crohn's disease.

Xu and Perler, 1996. EMBO J. 15(9), 5146-5153.

Wu C, et al., Nat Biotechnol. 2007 November; 25(11):1290-7. Simultaneous targeting of multiple disease mediators by a dual-variable-domain immunoglobulin.

Molecular Cloning: A Laboratory Manual, second edition (Sambrook et al., 1989); Oligonucleotide Synthesis (M. J. Gait, ed., 1984); Animal Cell Culture (R. I. Freshney, ed., 1987); Methods in Enzymology (Academic Press, Inc.); Handbook of Experimental Immunology (D. M. Weir & C. C. Blackwell, eds.); Gene Transfer Vectors for Mammalian Cells (J. M. Miller & M. P. Calos, eds., 1987); Current Protocols in Molecular Biology (F. M. Ausubel et al., eds., 1993); PCR: The Polymerase Chain Reaction, (Mullis et al., eds., 1994); and Current Protocols in Immunology (J. E. Coligan et al., eds., 1991).

We claim:

1. An isolated or purified expression vector for generating one or more recombinant protein products comprising a single open reading frame insert; said insert comprising:
(a) a signal peptide nucleic acid sequence encoding a signal peptide;
(b) a first nucleic acid sequence encoding a first polypeptide;
(c) a first intervening nucleic acid sequence encoding a first protein cleavage site, wherein said first protein cleavage site is provided by an intein segment of a lon protease gene of Pyrococcus or a klbA gene of Pyrococcus or Methanococcus, or a modified intein segment derived therefrom; and
(d) a second nucleic acid sequence encoding a second polypeptide;
wherein said first intervening nucleic acid sequence encoding said first protein cleavage site is operably positioned between said first nucleic acid sequence and said second nucleic acid sequence;
wherein said signal peptide nucleic acid sequence encoding said signal peptide is operably positioned before said first nucleic acid sequence; and
wherein said expression vector is capable of expressing a single open reading frame polypeptide cleavable at said first protein cleavage site.

2. The expression vector of claim 1 wherein said first protein cleavage site is provided by an intein segment of a lon protease gene of Pyrococcus abyssi, Pyrococcus furiosus, or Pyrococcus horikoshii OT3; or an intein segment of a klbA gene of Pyrococcus abyssi, Pyrococcus furiosus, or Methanococcus jannaschii; or a modified intein segment derived respectively therefrom.

3. The expression vector of claim 1 wherein the intein segment or modified intein segment encodes a penultimate residue which is a lysine, serine or not a histidine.

4. The expression vector of claim 1 wherein said intein segment or modified intein segment is capable of cleavage but not complete ligation of said first polypeptide to said second polypeptide.

5. The expression vector of claim 1 wherein said first protein cleavage site is provided by an intein segment comprising a sequence selected from the group consisting of SEQ ID NO: 1, 3, 4, 6, 7, 55, 35, 37, and 39 and modified intein segments derived therefrom.

6. The expression vector of claim 1 wherein the first polypeptide and second polypeptide are capable of multimeric assembly.

7. The expression vector of claim 1 wherein at least one of said first polypeptide and second polypeptide are capable of extracellular secretion.

8. The expression vector of claim 1 wherein at least one of said first polypeptide and second polypeptide are of mammalian origin.

9. The expression vector of claim 1 wherein said first polypeptide comprises an immunoglobulin heavy chain or functional fragment thereof, and said second polypeptide comprises an immunoglobulin light chain or functional fragment thereof, and said first polypeptide is upstream of said second polypeptide.

10. The expression vector of claim 1 comprising only one of said signal peptide nucleic acid sequence.

11. The expression vector of claim 1 further comprising a third nucleic acid sequence encoding a third polypeptide, and a second intervening nucleic acid sequence encoding a second protein cleavage site; wherein the second intervening nucleic acid sequence and third nucleic acid sequence, in that order, are operably positioned after said second nucleic acid sequence.

12. The expression vector of claim 1 wherein said first and said second polypeptide comprise a functional antibody or other antigen recognition molecule; with an antigen specificity directed to binding an antigen selected from the group consisting of: TNFα (tumor necrosis factor-alpha), erythropoietin receptor, RSV, EL/selectin, interleukin-1, interleukin-12, interleukin-13, interleukin-18, interleukin-23, interleukin-33, CD81, CD19, IGF1, IGF2, EGFR, CXCL-13, GLP-1R, prostaglandin E2, and amyloid beta.

13. The expression vector of claim 1 wherein the first and second polypeptides comprise a pair of immunoglobulin chains from an antibody of D2E7, EL246, ABT-007, ABT-325, or ABT-874.

14. The expression vector of claim 1, wherein the first and second polypeptide are each independently selected from an immunoglobulin heavy chain or an immunoglobulin light chain segment from an analogous segment of D2E7, EL246, ABT-007, ABT-325, ABT-874, or other antibody.

15. The expression vector of claim 1, wherein said vector further comprises a promoter regulatory element for said insert.

16. The expression vector according to claim 15, wherein said promoter regulatory element is inducible or constitutive.

17. The expression vector according to claim 15, wherein said promoter regulatory element is tissue specific.

18. The expression vector according to claim 15, wherein said promoter comprises an adenovirus major late promoter.

19. A host cell comprising a vector according to claim 1.

20. The host cell according to claim 19, wherein said host cell is a prokaryotic cell.

21. The host cell according to claim 20, wherein said host cell is Escherichia coli.

22. The host cell according to claim 19, wherein said host cell is a eukaryotic cell.

23. The host cell according to claim 22, wherein said eukaryotic cell is selected from the group consisting of a protist cell, animal cell, plant cell, and fungal cell.

24. The host cell according to claim 23, wherein said eukaryotic cell is an animal cell selected from the group consisting of a mammalian cell, an avian cell, and an insect cell.

25. The host cell according to claim 24, wherein said host cell is a mammalian cell line.

26. The host cell according to claim 24, wherein said host cell is a CHO cell or a dihydrofolate reductase-deficient CHO cell.

27. The host cell according to claim 24, wherein said host cell is a COS cell or HEK cell.

28. The host cell according to claim 23, wherein said host cell is a yeast cell.

29. The host cell according to claim 28, wherein said yeast cell is Saccharomyces cerevisiae.

30. The host cell according to claim 24, wherein said host cell is a Spodoptera frugiperda Sf9 insect cell.

31. A method for producing a recombinant polyprotein or a plurality of proteins, comprising culturing a host cell of claim 19 in a culture medium under conditions sufficient to allow expression of a vector protein.

32. The method of claim 31 further comprising recovering and/or purifying said vector protein.

33. The method of claim 31 wherein said plurality of proteins are capable of multimeric assembly.

34. The method of claim 31 wherein the recombinant polyprotein or plurality of proteins are biologically functional and/or therapeutic.

35. A method for producing a recombinant product, wherein the product is an immunoglobulin protein or functional fragment thereof, assembled antibody, or other antigen recognition molecule, comprising culturing a host cell according to claim 19 in a culture medium under conditions sufficient to produce the recombinant product.

36. A protein produced according to the method of claim 31.

37. A polyprotein produced according to the method of claim 31.

38. An assembled immunoglobulin; assembled other antigen recognition molecule; or individual immunoglobulin chain or functional fragment thereof produced according to the method of claim 31.

39. The immunoglobulin; other antigen recognition molecule; or individual immunoglobulin chain or functional fragment thereof according to claim 38, wherein there is a capability to effect or contribute to specific antigen binding to tumor necrosis factor-α, erythropoietin receptor, RSV, EL/selectin, interleukin-1, interleukin-12, interleukin-13, interleukin-18, interleukin-23, interleukin-33, CD81, CD19, IGF1, IGF2, EGFR, prostaglandin E2, CXCL-13, GLP-1R, or amyloid beta.

40. The immunoglobulin or functional fragment thereof according to claim 39, wherein the immunoglobulin is D2E7 or ABT-874 or wherein the functional fragment is a fragment respectively thereof.

41. A pharmaceutical composition comprising a therapeutically effective amount of a protein according to claim 36, and a pharmaceutically acceptable carrier.

42. The expression vector of claim 1, further comprising a nucleic acid sequence encoding a tag.

43. The expression vector of claim 1, wherein said intervening nucleic acid sequence additionally encodes a tag.

44. An expression vector, host cell with the vector, vector expression product, pharmaceutical composition, and/or method of making or using of any of the foregoing, wherein the vector is the vector of claim 1 and further comprises a segment encoding a light chain signal peptide.

45. The vector, host cell, vector expression product, pharmaceutical composition and/or method of making or using of claim 44 wherein the encoded light chain signal peptide is a kappa light chain signal peptide selected from the group consisting of A17, A18, A19, A26, and H2G.

46. The vector, host cell, vector expression product, pharmaceutical composition and/or method of making or using of claim 44 wherein the encoded light chain signal peptide is VKII kappa light chain signal peptide A18, SEQ ID NO:82 (amino acid sequence MRLPAQLLGLLMLWIPGSSA).


SEQUENCE LISTING (108 sequences)

SEQ ID NO: 1 - protein, 335 aa, Pyrococcus abyssi
QCFSGEETVVIRENGEVKVLRLKDFVEKALEKPSGEGLDGDVKVVYHDFRNENVEVLTKDGFTKLLYANKRIGKQKLRRVVNLEKDYWFALTPDHKVYTTDGLKEAGEITEKDELISVPITVFDCEDEDLKKIGLLPLTSDDERLRKIATLMGILFNGGSIDEGLGVLTLKSERSVIEKFVITLKELFGKFEYEIIKEENTILKTRDPRIIKFLVGLGAPIEGKDLKMPWWVKLKPSLFLAFLEGFRAHIVEQLVDDPNKNLPFFQELSWYLGLFGIKADIKVEEVGDKHKIIFDAGRLDVDKQFIETWEDVEVTYNLTTEKGNLLANGLFVKNS

SEQ ID NO: 2 - DNA, 999 bp, Pyrococcus abyssi
tgcttcagcggcgaggaaaccgtggtgatccgggagaacggcgaggtgaaggtgctgcggctgaaggacttcgtggagaaggccctggaaaagccctccggcgagggcctggacggcgacgtgaaagtggtgtaccacgacttccggaacgagaacgtggaggtgctgaccaaggacggcttcaccaagctgctgtacgccaacaagcggatcggcaagcagaaactgcggcgggtggtgaacctggaaaaggactactggttcgccctgacccccgaccacaaggtgtacaccaccgacggcctgaaagaggccggcgagatcaccgagaaggacgagctgatcagcgtgcccatcaccgtgttcgactgcgaggacgaggacctgaagaagatcggcctgctgcccctgaccagcgacgacgagcggctgcggaagatcgccaccctgatgggcatcctgttcaacggcggcagcatcgatgagggcctgggcgtgctgaccctgaagagcgagcggagcgtgatcgagaagttcgtgatcaccctgaaagagctgttcggcaagttcgagtacgagatcatcaaagaggaaaacaccatcctgaaaacccgggacccccggatcatcaagtttctggtgggcctgggagcccccatcgagggcaaggatctgaagatgccttggtgggtgaagctgaagcccagcctgttcctggccttcctggaaggcttccgggcccacatcgtggagcagctggtcgacgaccccaacaagaatctgcccttctttcaggaactgagctggtatctgggcctgttcggcatcaaggccgacatcaaggtggaggaagtgggcgacaagcacaagatcatcttcgacgccggcaggctggacgtggacaagcagttcatcgagacctgggaggatgtggaggtgacctacaacctgaccacagagaagggcaatctgctggccaacggcctgttcgtgaagaac

SEQ ID NO: 3 - protein, 333 aa, Pyrococcus abyssi
CFSGEETVVIRENGEVKVLRLKDFVEKALEKPSGEGLDGDVKVVYHDFRNENVEVLTKDGFTKLLYANKRIGKQKLRRVVNLEKDYWFALTPDHKVYTTDGLKEAGEITEKDELISVPITVFDCEDEDLKKIGLLPLTSDDERLRKIATLMGILFNGGSIDEGLGVLTLKSERSVIEKFVITLKELFGKFEYEIIKEENTILKTRDPRIIKFLVGLGAPIEGKDLKMPWWVKLKPSLFLAFLEGFRAHIVEQLVDDPNKNLPFFQELSWYLGLFGIKADIKVEEVGDKHKIIFDAGRLDVDKQFIETWEDVEVTYNLTTEKGNLLANGLFVKN

SEQ ID NO: 4 - protein, 403 aa, Pyrococcus furiosus
QCFSGEEVILIEKDGEKKVFKLREFVDGLLKEASGEGMDGSIRVVYKDLQGENIKILTKDGLVKLLYVNRREGKQKLRKIVNLEKDYWLALTPEHKVYTIKGLKEAGEITKDDEIIRVPLTILDGFDVAEKSIREELERLSLLPLNSEDSRLEKIAGIMGALFGSGGIDENLNTLSFVSSEKKTIEQFVKALSELFGEFDYKIEEKENSIIFRTCDKRIVTFFATLGAPVGDKSKVKLKLPWWVKLKPSLFLAFMDGLYSSNRNDKEILEITQLTDNVETFFEEISWYLSFFGIKAEAEEDEEKDKYRARLTLSSSIDNMLNFIEFIPISFSPAKREKFFKEIEKYLEYSIPEKTEDLKKRVKRVKKGERRNFLESWEEVEVTYNVTTETGNLLANGLFVKNS

SEQ ID NO: 5 - DNA, 1203 bp, Pyrococcus furiosus
tgttttagcggtgaagaagttatcttaattgaaaaggacggagagaaaaaagtcttcaaacttagggagttcgttgacggtctccttaaggaggcgtctggagaagggatggacggaagtattagagtagtttataaagatcttcaaggggaaaacataaaaatactcacaaaagacggacttgtaaagctcctttatgtcaatagaagagaagggaagcaaaagcttagaaaaatagtaaatcttgaaaaggattattggcttgcattaacacctgaacataaagtgtacacaataaagggccttaaagaagctggagagataactaaagatgatgagataataagagtgcctctcacaattcttgacggctttgacgtagccgagaagagtataagagaggaacttgaaaggcttagcctacttccactaaatagtgaagacagtagactagaaaagatagcaggaatcatgggcgcactctttggtagtggaggtatcgatgagaatctcaatacccttagctttgtttctagcgagaagaaaacaattgaacagtttgttaaagcactcagcgagctcttcggggaatttgactataaaattgaagaaaaagaaaacagcattattttcagaacatgtgataaaagaatagtgaccttctttgctacacttggtgcaccagttggagacaaaagcaaagttaagcttaagcttccatggtgggtcaagcttaagccgtcacttttcctcgccttcatggatggtctctacagtagcaataggaatgacaaagaaatcctcgaaataactcaacttactgacaacgtcgaaacgttcttcgaggaaatatcttggtatctgagcttctttggaattaaggcagaagctgaagaggatgaagaaaaagataaatacagggctagacttacgctatcctcatcaatagacaacatgcttaatttcattgagttcattccaataagcttttctccagcaaagagagaaaaattctttaaggaaattgaaaaatatctggaatatagcattcccgaaaagactgaggatcttaagaaacgagttaagagagttaagaagggagagagaaggaatttcctcgaaagctgggaggaagttgaagttacttacaacgtaactacagagacaggaaatctacttgctaacggtctatttgttaagaac

SEQ ID NO: 6 - protein, 401 aa, Pyrococcus furiosus
CFSGEEVILIEKDGEKKVFKLREFVDGLLKEASGEGMDGSIRVVYKDLQGENIKILTKDGLVKLLYVNRREGKQKLRKIVNLEKDYWLALTPEHKVYTIKGLKEAGEITKDDEIIRVPLTILDGFDVAEKSIREELERLSLLPLNSEDSRLEKIAGIMGALFGSGGIDENLNTLSFVSSEKKTIEQFVKALSELFGEFDYKIEEKENSIIFRTCDKRIVTFFATLGAPVGDKSKVKLKLPWWVKLKPSLFLAFMDGLYSSNRNDKEILEITQLTDNVETFFEEISWYLSFFGIKAEAEEDEEKDKYRARLTLSSSIDNMLNFIEFIPISFSPAKREKFFKEIEKYLEYSIPEKTEDLKKRVKRVKKGERRNFLESWEEVEVTYNVTTETGNLLANGLFVKN

SEQ ID NO: 7 - protein, 333 aa, Pyrococcus abyssi; identical to SEQ ID NO: 3

SEQ ID NO: 8 - DNA, 999 bp, Pyrococcus abyssi; identical to SEQ ID NO: 2

SEQ ID NO: 9 - protein, 10 aa, Artificial Sequence
  Synthetic construct
EVQLVESGGG

SEQ ID NO: 10 - protein, 10 aa, Artificial Sequence
  Synthetic construct
MEVQLVESGG

SEQ ID NO: 11 - protein, 10 aa, Artificial Sequence
  Synthetic construct
DIQMTQSPSS

SEQ ID NO: 12 - protein, 10 aa, Artificial Sequence
  Synthetic construct
MDIQMTQSPS

SEQ ID NO: 13 - protein, 8 aa, Artificial Sequence
  Synthetic construct
ANGLFVKN

SEQ ID NO: 14 - protein, 5 aa, Artificial Sequence
  Synthetic construct
MRAKR

SEQ ID NO: 15 - protein, 8 aa, Artificial Sequence
  Synthetic construct
HARGVFRR

SEQ ID NO: 16 - protein, 8 aa, Artificial Sequence
  Synthetic construct
MDRGVFRR

SEQ ID NO: 17 - protein, 7 aa, Artificial Sequence
  Synthetic construct
DIQMTQS

SEQ ID NO: 18 - protein, 7 aa, Artificial Sequence
  Synthetic construct
AIQMTQS

SEQ ID NO: 19 - protein, 7 aa, Artificial Sequence
  Synthetic construct
NIQMTQS

SEQ ID NO: 20 - protein, 7 aa, Artificial Sequence
  Synthetic construct
NFQMTQS

SEQ ID NO: 21 - protein, 8 aa, Artificial Sequence
  Synthetic construct
MDIQMTQS

SEQ ID NO: 22 - protein, 12 aa, Artificial Sequence
  Synthetic construct
MRAKRDIQMTQS

SEQ ID NO: 23 - protein, 9 aa, Artificial Sequence
  Synthetic construct
YPDIQMTQS

SEQ ID NO: 24 - protein, 9 aa, Artificial Sequence
  Synthetic construct
RPDIQMTQS

SEQ ID NO: 25 - protein, 9 aa, Artificial Sequence
  Synthetic construct
VPDIQMTQS

SEQ ID NO: 26 - protein, 9 aa, Artificial Sequence
  Synthetic construct
QPDIQMTQS

SEQ ID NO: 27 - protein, 9 aa, Artificial Sequence
  Synthetic construct
APDIQMTQS

SEQ ID NO: 28 - protein, 9 aa, Artificial Sequence
  Synthetic construct
HADIQMTQS

SEQ ID NO: 29 - protein, 9 aa, Artificial Sequence
  Synthetic construct
YADIQMTQS

SEQ ID NO: 30 - protein, 9 aa, Artificial Sequence
  Synthetic construct
MPDIQMTQS

SEQ ID NO: 31 - protein, 9 aa, Artificial Sequence
  Synthetic construct
MADIQMTQS

SEQ ID NO: 32 - protein, 15 aa, Artificial Sequence
  Synthetic construct
HARGVFRRDIQMTQS

SEQ ID NO: 33 - protein, 15 aa, Artificial Sequence
  Synthetic construct
MDRGVFRRDIQMTQS

SEQ ID NO: 34 - DNA, 504 bp, Methanococcus jannaschii
gctctggcctacgacgagcccatctacctgagcgacggcaacatcatcaacatcggcgagttcgtggacaagttcttcaagaagtacaagaacagcatcaagaaagaggacaacggcttcggctggatcgacatcggcaacgagaacatctacatcaagagcttcaacaagctgtccctgatcatcgaggacaagcggatcctgagagtgtggcggaagaagtacagcggcaagctgatcaagatcaccaccaagaaccggcgggagatcaccctgacccacgaccaccccgtgtacatcagcaagaccggcgaggtgctggaaatcaacgccgagatggtgaaagtgggcgactacatctatatccccaagaacaacaccatcaacctggacgaggtgatcaaggtggagaccgtggactacaacggccacatctacgacctgaccgtggaggacaaccacacctacatcgccggcaagaacgagggcttcgccgtgagcaac

SEQ ID NO: 35 - protein, 168 aa, Methanococcus jannaschii
ALAYDEPIYLSDGNIINIGEFVDKFFKKYKNSIKKEDNGFGWIDIGNENIYIKSFNKLSLIIEDKRILRVWRKKYSGKLIKITTKNRREITLTHDHPVYISKTGEVLEINAEMVKVGDYIYIPKNNTINLDEVIKVETVDYNGHIYDLTVEDNHTYIAGKNEGFAVSN

SEQ ID NO: 36 - DNA, 588 bp, Pyrococcus abyssi
gctctgtactacttcagcgagatccagctgcccaacggcaaagagttcatcggcaaactggtggacgagctgttcgagaagtaccacgacaagatcggcaagtacaaggacatggaatacgtggagctgaacgaagaggacaccttcgaggtgatcagcatcggccccgacctgagcgccaggcggcacaaggtgacccacgtgtggcggcggaaggtgaaagacggcgagaagctggtgaagatccggaccgccagcggcaaagaactggtgctgacccaggaccaccccgtgttcgtgctgctgggccgggacgtggccagacgggacgccggcaacgtgaaagtgggcgacgagatcgccgtgctgaacaccaggcccgacttcagcgtgctgtccccccctgccatgcccgagctgctgtccgagcccttcaactacgagctgtccagcatcggcgacgtggcctgggacgaggtggtggaggtggacgagatcgacgccaagggcctgggcgtggagtacctgtacgacctgaccgtggacatcaaccacaactacgtggccaacggcatcgtggtgtccaac

SEQ ID NO: 37 - skipped

SEQ ID NO: 38 - DNA, 1566 bp, Pyrococcus furiosus
gcactttacgatttctctgtcatccaactatctaatggtagatttgtacttataggagatttagtcgaggaattattcaagaagtatgccgagaaaattaaaacatacaaagaccttgagtacatagagcttaacgaggaagaccgttttgaagttgttagtgttagtccagatttgaaggctaataaacatgttgtctcaagagtttggagaagaaaggtcagagagggggaaaagctaatacgcataaagacgagaactggcaacgaaataatcctcactagaaatcatccgctatttgccttctccaatggagacgtagtcagaaaagaggccgagaagctcaaagttggggatagagttgcagtgatgatgagacctccttcacctcctcaaactaaagctgtagttgaccctgcaatttacgtgaaaataagtgattactaccttgttccgaacggaaaaggtatgataaaagttcctaacgatggtattcctccagaaaaggcccaatatcttctttcagtaaattcatatcctgtaaaattagtcagagaagttgatgagaagttatcctatctcgctggagttatactcggtgatgggtatatatcatcgaatggatactacatctcagctacatttgacgacgaagcttacatggatgcctttgtctctgtagtctcggactttatccctaactatgtccccagtataaggaagaacggagattacacaattgtaactgttggctcgaagatttttgctgaaatgctctcaaggatatttggaataccaaggggcagaaaatctatgtgggatattccagacgtagtactttcaaatgacgatcttatgagatacttcatagctggacttttcgacgctgatgggtacgtagatgaaaatgggccctccatagtcctagtaacaaagagtgaaaccgtggcaaggaagatttggtacgttcttcagaggttggggatcataagtacagtttcccgtgtaaagagcagagggtttaaagaaggcgagctgttcagggtaattattagtggtgttgaagatcttgctaaatttgcaaaattcatacccctacgtcactcaagaaagagggccaaacttatggagatattaaggactaagaagccatatcggggaagaagaacttaccgcgtgccgatatccagtgatatgatagctcctctccgtcaaatgttgggattaactgttgcagagctgtctaagttagcgtcttattatgcaggggaaaaagtttctgaaagcctaattaggcatatagaaaagggaagggtcaaagagataagacgctctacgctcaaggggattgcccttgctctccagcagatagctaaagatgtgggtaacgaagaagcttgggtgagagccaagaggcttcaattgatagctgagggagatgtttactgggatgaagtcgtaagtgttgaggaagttgatccgaaggagcttggcattgagtacgtctatgacctcacggttgaggacgaccacaattatgtggcaaatggcatactagtctcaaac

SEQ ID NO: 39 - protein, 522 aa, Pyrococcus furiosus
ALYDFSVIQLSNGRFVLIGDLVEELFKKYAEKIKTYKDLEYIELNEEDRFEVVSVSPDLKANKHVVSRVWRRKVREGEKLIRIKTRTGNEIILTRNHPLFAFSNGDVVRKEAEKLKVGDRVAVMMRPPSPPQTKAVVDPAIYVKISDYYLVPNGKGMIKVPNDGIPPEKAQYLLSVNSYPVKLVREVDEKLSYLAGVILGDGYISSNGYYISATFDDEAYMDAFVSVVSDFIPNYVPSIRKNGDYTIVTVGSKIFAEMLSRIFGIPRGRKSMWDIPDVVLSNDDLMRYFIAGLFDADGYVDENGPSIVLVTKSETVARKIWYVLQRLGIISTVSRVKSRGFKEGELFRVIISGVEDLAKFAKFIPLRHSRKRAKLMEILRTKKPYRGRRTYRVPISSDMIAPLRQMLGLTVAELSKLASYYAGEKVSESLIRHIEKGRVKEIRRSTLKGIALALQQIAKDVGNEEAWVRAKRLQLIAEGDVYWDEVVSVEEVDPKELGIEYVYDLTVEDDHNYVANGILVSN

SEQ ID NO: 40 - protein, 4 aa, Artificial Sequence
  Synthetic construct
GHDG

SEQ ID NO: 41 - protein, 4 aa, Artificial Sequence
  Synthetic construct
SPGK

SEQ ID NO: 42 - protein, 4 aa, Artificial Sequence
  Synthetic construct
ALYY

SEQ ID NO: 43 - protein, 4 aa, Artificial Sequence
  Synthetic construct
CLYY

SEQ ID NO: 44 - protein, 4 aa, Artificial Sequence
  Synthetic construct
CMGT

SEQ ID NO: 45 - protein, 4 aa, Artificial Sequence
  Synthetic construct
MDIQ

SEQ ID NO: 46 - protein, 7 aa, Artificial Sequence
  Synthetic construct
LSLSPGK

SEQ ID NO: 47 - protein, 6 aa, Artificial Sequence
  Synthetic construct
LSLSPG

SEQ ID NO: 48 - protein, 9 aa, Artificial Sequence
  Synthetic construct
ALYYFSEIQ

SEQ ID NO: 49 - DNA, 44 bp, Artificial Sequence
  Synthetic construct
gcctctccctgtctccgggtgctctgtactacttcagcgagatc

SEQ ID NO: 50 - DNA, 44 bp, Artificial Sequence
  Synthetic construct
gcctctccctgtctccgggttgtctgtactacttcagcgagatc

SEQ ID NO: 51 - DNA, 44 bp, Artificial Sequence
  Synthetic construct
tctccctgtctccgggtaaatgtctgtactacttcagcgagatc

SEQ ID NO: 52 - DNA, 20 bp, Artificial Sequence
  Synthetic construct
cggcgtggaggtgcataatg

SEQ ID NO: 53 - DNA, 18 bp, Artificial Sequence
  Synthetic construct
acccggagacagggagag

SEQ ID NO: 54 - DNA, 20 bp, Artificial Sequence
  Synthetic construct
gggtcagcaccagttctttg

SEQ ID NO: 55 - protein, 476 aa, Pyrococcus horikoshii
QCFSGEEVIIVEKGKDRKVVKLREFVEDALKEPSGEGMDGDIKVTYKDLRGEDVRILTKDGFVKLLYVNKREGKQKLRKIVNLDKDYWLAVTPDHKVFTSEGLKEAGEITEKDEIIRVPLVILDGPKIASTYGEDGKFDDYIRWKKYYEKTGNGYKRAAKELNIKESTLRWWTQGAKPNSLKMIEELEKLNLLPLTSEDSRLEKVAIILGALFSDGNIDRNFNTLSFISSERKAIERFVETLKELFGEFNYEIRDNHESLGKSILFRTWDRRIIRFFVALGAPVGNKTKVKLELPWWIKLKPSLFLAFMDGLYSGDGSVPRFARYEEGIKFNGTFEIAQLTDDVEKKLPFFEEIAWYLSFFGIKAKVRVDKTGDKYKVRLIFSQSIDNVLNFLEFIPISLSPAKREKFLREVESYLAAVPESSLAGRIEELREHFNRIKKGERRSFIETWEVVNVTYNVTTETGNLLANGLFVKNS

SEQ ID NO: 56 - protein, 6 aa, Artificial Sequence
  Synthetic construct
HHHHHH

SEQ ID NO: 57 - protein, 10 aa, Artificial Sequence
  Synthetic construct
HHHHHHHHHH

SEQ ID NO: 58 - protein, 6 aa, Artificial Sequence
  Synthetic construct
HQHQHQ

SEQ ID NO: 59 - protein, 22 aa, Artificial Sequence
  Synthetic construct
MDMRVPAQLLGLLLLWLRGARC

SEQ ID NO: 60 - protein, 22 aa, Artificial Sequence
  Synthetic construct
MDMRVPAQLLGLLLLWLRGARC

SEQ ID NO: 61 - protein, 22 aa, Artificial Sequence
  Synthetic construct
MDMRVPAQLLGLLQLWLSGARC

SEQ ID NO: 62 - protein, 22 aa, Artificial Sequence
  Synthetic construct
MDMRVPAQLLGLLLLWLSGARC

SEQ ID NO: 63 - protein, 22 aa, Artificial Sequence
  Synthetic construct
MDMRVPAQLLGLLLLWLPDTRC

SEQ ID NO: 64 - protein, 22 aa, Artificial Sequence
  Synthetic construct
MDMRVPAQLLGLLLLWFPGARC

SEQ ID NO: 65 - protein, 22 aa, Artificial Sequence
  Synthetic construct
MDMRVPAQLLGLLLLWFPGARC

SEQ ID NO: 66 - protein, 22 aa, Artificial Sequence
  Synthetic construct
MDMRVLAQLLGLLLLCFPGARC

SEQ ID NO: 67 - protein, 22 aa, Artificial Sequence
  Synthetic construct
MDMRVLAQLLGLLLLCFPGARC

SEQ ID NO: 68 - protein, 22 aa, Artificial Sequence
  Synthetic construct
MDMRVPAQLLGLLLLWLPGARC

SEQ ID NO: 69 - protein, 22 aa, Artificial Sequence
  Synthetic construct
MDMRVPAQLLGLLLLWLPGARC

SEQ ID NO: 70 - protein, 22 aa, Artificial Sequence
  Synthetic construct
MDMRVPAQLLGLLLLWFPGSRC

SEQ ID NO: 71 - protein, 22 aa, Artificial Sequence
  Synthetic construct
MDMRVPAQLLGLLLLWFPGSRC

SEQ ID NO: 72 - protein, 22 aa, Artificial Sequence
  Synthetic construct
MDMRVPAQLLGLLLLWLPGARC

SEQ ID NO: 73 - protein, 22 aa, Artificial Sequence
  Synthetic construct
MDMRVPAQRLGLLLLWFPGARC

SEQ ID NO: 74 - protein, 20 aa, Artificial Sequence
  Synthetic construct
MRVPAQLLGLLLLWLPGARC

SEQ ID NO: 75 - protein, 22 aa, Artificial Sequence
  Synthetic construct
MDMRVPAQLLGLLLLWLPGARC

SEQ ID NO: 76 - protein, 22 aa, Artificial Sequence
  Synthetic construct
MDMRVPAQLLGLLLLWLPGARC

SEQ ID NO: 77 - protein, 22 aa, Artificial Sequence
  Synthetic construct
MDMRVPAQLLGLLLLWLPGAKC

SEQ ID NO: 78 - protein, 20 aa, Artificial Sequence
  Synthetic construct
MRLPAQLLGLLMLWVPGSSE

SEQ ID NO: 79 - protein, 20 aa, Artificial Sequence
  Synthetic construct
MRLPAQLLGLLMLWVPGSSE

SEQ ID NO: 80 - protein, 20 aa, Artificial Sequence
  Synthetic construct
MRLPAQLLGLLMLWVPGSSG

SEQ ID NO: 81 - protein, 20 aa, Artificial Sequence
  Synthetic construct
MRLPAQLLGLLMLWVPGSSG

SEQ ID NO: 82 - protein, 20 aa, Artificial Sequence
  Synthetic construct
MRLPAQLLGLLMLWIPGSSA

SEQ ID NO: 83 - protein, 20 aa, Artificial Sequence
  Synthetic construct
MRLPAQLLGLLMLWIPGSSA

SEQ ID NO: 84 - protein, 20 aa, Artificial Sequence
  Synthetic construct
MRLPAQLLGLLMLWVSGSSG

SEQ ID NO: 85 - protein, 20 aa, Artificial Sequence
  Synthetic construct
MRLPAQLLGLLMLWVSGSSG

SEQ ID NO: 86 - protein, 20 aa, Artificial Sequence
  Synthetic construct
MRLLAQLLGLLMLWVPGSSG

SEQ ID NO: 87 - protein, 20 aa, Artificial Sequence
  Synthetic construct
METPAQLLFLLLLWLPDTTG

SEQ ID NO: 88 - protein, 20 aa, Artificial Sequence
  Synthetic construct
METPAQLLFLLLLWLPDTTG

SEQ ID NO: 89 - protein, 20 aa, Artificial Sequence
  Synthetic construct
MEAPAQLLFLLLLWLPDTTG

SEQ ID NO: 90 - protein, 20 aa, Artificial Sequence
  Synthetic construct
MEAPAQLLFLLLLWLPDTTG

SEQ ID NO: 91 - protein, 20 aa, Artificial Sequence
  Synthetic construct
MEAPAQLLFLLLLWLPDTTG

SEQ ID NO: 92 - protein, 20 aa, Artificial Sequence
  Synthetic construct
MEAPAQLLFLLLLWLTDTTG

SEQ ID NO: 93 - protein, 23 aa, Artificial Sequence
  Synthetic construct
MEPWKPQHSFFFLLLLWLPDTTG

SEQ ID NO: 94 - protein, 20 aa, Artificial Sequence
  Synthetic construct
MVLQTQVFISLLLWISGAYG

SEQ ID NO: 95 - protein, 20 aa, Artificial Sequence
  Synthetic construct
MGSQVHLLSFLLLWISDTRA

SEQ ID NO: 96 - protein, 19 aa, Artificial Sequence
  Synthetic construct
MLPSQLIGFLLLWVPASRG

SEQ ID NO: 97 - protein, 19 aa, Artificial Sequence
  Synthetic construct
MLPSQLIGFLLLWVPASRG

SEQ ID NO: 98 - protein, 20 aa, Artificial Sequence
  Synthetic construct
MVSPLQFLRLLLLWVPASRG

SEQ ID NO: 99 - protein, 22 aa, Artificial Sequence
  Synthetic construct
MDMRVPAQLLGLLLLWFPGSGG

SEQ ID NO: 100 - protein, 23 aa, Artificial Sequence
  Synthetic construct
MDMRVPAQLLGLLLLWFPGSGGG

SEQ ID NO: 101 - protein, 24 aa, Artificial Sequence
  Synthetic construct
MDMRVPAQLLGLLLLWFPGSGGGG

SEQ ID NO: 102 - protein, 25 aa, Artificial Sequence
  Synthetic construct
MDMRVPAQLLGLLLLWFPGSGGGGG

SEQ ID NO: 103 - protein, 22 aa, Artificial Sequence
  Synthetic construct
MRMRVPAQLLGLLLLWFPGSRC

SEQ ID NO: 104 - protein, 22 aa, Artificial Sequence
  Synthetic construct
MRMRVPAQLLGLLLLWFPGSGG

SEQ ID NO: 105 - protein, 23 aa, Artificial Sequence
  Synthetic construct
MRRMRVPAQLLGLLLLWFPGSRC

SEQ ID NO: 106 - protein, 23 aa, Artificial Sequence
  Synthetic construct
MRRMRVPAQLLGLLLLWFPGSGG

SEQ ID NO: 107 - protein, 24 aa, Artificial Sequence
  Synthetic construct
MRRRMRVPAQLLGLLLLWFPGSGG

SEQ ID NO: 108 - protein, 20 aa, Artificial Sequence
  Synthetic construct
MDMRVPAQLLGDEWFPGSGG